(19) United States
(12) Patent Application Publication
(10) Pub. No.: US 2023/0024833 A1
(43) Pub. Date: Jan. 26, 2023

(54) SPLIT DEAMINASE BASE EDITORS

(71) Applicant: The General Hospital Corporation, Boston, MA (US)

(72) Inventors: J. Keith Joung, Winchester, MA (US); James Angstman, Boston, MA (US)

(21) Appl. No.: 17/781,500

(22) PCT Filed: Dec. 4, 2020
(86) PCT No.: PCT/US2020/063264

§ 371 Date: Jun. 1, 2022

Related U.S. Application Data
(60) Provisional application No. 62/944,897, filed on Dec. 6, 2019.

(51) Int. Cl. C12N 9/78; C12N 9/22; C12N 15/11; C12N 15/62; C12N 5/0789; C07K 14/00

(52) U.S. Cl. CPC C12N 9/78; C12Y 305/04; C12N 9/22; C12N 15/11; C12N 15/625; C12N 5/0647; C07K 14/001; C12N 2310/20; C12N 2800/80; C07K 2319/02

(57) ABSTRACT

Provided herein are compositions and methods for improving the genome-wide specificities of targeted base editing technologies.

### CLAIM OF PRIORITY

This application claims the benefit of U.S. Provisional Application Ser. No. 62/944,897 filed on Dec. 6, 2019. The entire contents of the foregoing are incorporated herein by reference.

### FEDERALLY SPONSORED RESEARCH OR DEVELOPMENT

This invention was made with Government support under Grant Nos. GM118158 and HG009490 awarded by the National Institutes of Health. The Government has certain rights in the invention.

### TECHNICAL FIELD

The present application is related to nucleic acid base editing technologies.

### BACKGROUND

Base editing (BE) technologies use an engineered DNA binding domain (such as RNA-guided, catalytically inactive Cas9 (dead Cas9 or dCas9), a nickase version of Cas9 (nCas9), or zinc finger (ZF) arrays) to recruit a cytidine deaminase domain to a specific genomic location to effect site-specific substitutions, e.g., cytosine→thymine (C→T) transition substitutions. See Komor et al., “Programmable Editing of a Target Base in Genomic DNA without Double-stranded DNA Cleavage.” Nature 533.7603 (2016): 420-24 and Yang et al. “Engineering and Optimising Deaminase Fusions for Genome Editing.” Nature Communications 7 (2016): 13330. BE is a particularly attractive tool for treating genetic diseases that manifest in cellular contexts where making precise mutations by homology directed repair (HDR) would be therapeutically beneficial but are difficult to create with traditional nuclease-based genome editing technology. For example, it is challenging or impossible to achieve HDR outcomes in tissues composed primarily of slowly dividing or post-mitotic cell populations, since HDR pathways are restricted to the G2 and S phases of the cell cycle. See Jasin, Maria, and Rodney Rothstein. “Repair of strand breaks by homologous recombination.” Cold Spring Harbor Perspectives in Biology 5.11 (2013): a012740. In addition, the efficiency of HDR repair can be substantially degraded before and after the edits are created by the competing and more efficient induction of variable-length indel mutations caused by non-homologous end-joining-mediated repair of nuclease-induced breaks. By contrast, BE technology has the potential to allow practitioners to make highly controllable, highly precise mutations without the need for cell-type-variable DNA repair mechanisms.

### SUMMARY

Base editor platforms (BE) possess the unique capability to generate precise, user-defined genome-editing events without the need for a donor DNA molecule. Base Editors (BEs) that include a single strand nicking CRISPR-Cas9 (nCas9) protein fused to cytosine deaminase domain and uracil glycosylase inhibitor (UGI) domains (e.g., BE3) efficiently induce cytosine-to-thymine (C-to-T) base transitions in a site-specific manner as determined by the CRISPR guide RNA (gRNA) spacer sequence1. As with all genome editing reagents, it is critical to first determine and then mitigate BE's capacity for generating off-target mutations before it is used for therapeutics so as to limit its potential for creating deleterious and irreversible genetically-encoded side-effects.

Thus, herein we describe a split-deaminase base editor (sDA-BE) comprising (i) a first fusion protein comprising a first nuclear localization signal (NLS) and a catalytically inactive or catalytically deficient N-terminal portion of a deaminase enzyme, but not a programmable DNA binding domain; and (ii) a second fusion protein comprising a second nuclear localization signal (NLS), a catalytically inactive or catalytically deficient C-terminal portion of the deaminase enzyme, and a programmable DNA binding domain, wherein the first fusion protein and second fusion protein, when co-expressed, form a catalytically active deaminase enzyme.

In some embodiments, the second fusion protein further comprises an N-terminal methionine.

In some embodiments, the second fusion protein further comprises one or more UGI sequences.

In some embodiments, the deaminase enzyme is selected from the group consisting of hAID, rAPOBEC1, mAPOBEC3, hAPOBEC3A, hAPOBEC3B, hAPOBEC3C, hAPOBEC3F, hAPOBEC3G, hAPOBEC3H, and variants thereof.

In some embodiments, the programmable DNA binding domain is selected from the group consisting of zinc fingers (ZFs), transcription activator effector-like effectors (TALEs), and Clustered Regularly Interspaced Short Palindromic Repeats (CRISPR) Cas RNA-guided nucleases (RGNs), catalytically inactive Cas9 (dCas9) nicking Cas9 (nCas9), and variants thereof.

Also described herein is a split-deaminase base editor (sDA-BE) comprising (i) a first fusion protein comprising an amino acid sequence selected from the group consisting of amino acids 1-90 of SEQ ID NO:1, amino acids 1-92 of SEQ ID NO:1, SEQ ID NO:8, SEQ ID NO:9; SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, or SEQ ID NO:26; and (ii) a second fusion protein comprising an amino acid sequence selected from the group consisting of amino acids 101-1,853 of SEQ ID NO:1, amino acids 97-1,853 of SEQ ID NO:1, SEQ ID NO:5, SEQ ID NO:6, SEQ ID NO:7, SEQ ID NO: 11, or SEQ ID NO:69.

Also described herein is a split-deaminase base editor (sDA-BE) comprising (i) a first fusion protein comprising a first nuclear localization signal (NLS), a catalytically inactive or catalytically deficient N-terminal portion of a deaminase enzyme, and a single strand nickase; and (ii) a second fusion protein comprising a second nuclear localization signal (NLS), a catalytically inactive or catalytically deficient C-terminal portion of a deaminase enzyme, and a programmable DNA binding domain, wherein the first fusion protein and the second fusion protein, when co-expressed, form a catalytically active deaminase enzyme.

In some embodiments, the first fusion protein comprises the amino acid sequence of SEQ ID NO:29, and wherein the second fusion protein comprises an amino acid sequence selected from the group consisting of SEQ ID NO:5 and SEQ ID NO:11.

Also described herein are nucleic acid(s) encoding any one or more of the fusion proteins described herein.

Also described herein are composition(s) comprising one or more nucleic acids, collectively encoding each of the fusion proteins of the sDA-BE described herein.

Also described herein are composition(s) comprising one or more nucleic acid expression vector(s) comprising the nucleic acid(s) described herein.

Also described herein are cell(s) comprising one or more nucleic acid expression vector(s) comprising the nucleic acid(s) described herein.

In some embodiments, the cell is an isolated host cell. In some embodiments, the cell is a stem cell. In some embodiments, the stem cell is a hematopoietic stem cell.

Also described herein are methods of targeted deamination of a nucleic acid comprising contacting the nucleic acid with any of the split deaminase base editor(s) (sDA-BE) described herein and, if the programmable DNA binding domain is a CRISPR based programmable DNA binding domain, contacting the nucleic acid with a gRNA.

Also described herein are methods of targeted deamination of a nucleic acid in a cell comprising expressing the nucleic acid(s) described herein in the cell and, if the programmable DNA binding domain is a CRISPR based programmable DNA binding domain, expressing a gRNA in the cell.

In some embodiments, the cell is a eukaryotic cell. In some embodiments, the cell is a mammalian cell. In some embodiments, the cell is a human cell.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 shows on-target editing rates of sDA-BE-TT at the EMX1-1 target site, including C-to-T editing, C-to-R editing, and indel formation.

FIG. 2 shows on-target editing rates of sDA-BE-TT at the EMX1-2 target site, including C-to-T editing, C-to-R editing, and indel formation.

FIG. 3 shows on-target editing rates of sDA-BE-TT at the FANCF target site, including C-to-T editing, C-to-R editing, and indel formation.

FIG. 4 shows on-target editing rates of sDA-BE-TT at the HEK Site 2 target site, including C-to-T editing, C-to-R editing, and indel formation.

FIG. 5 shows on-target editing rates of sDA-BE-TT at the HEK Site 3 target site, including C-to-T editing, C-to-R editing, and indel formation.

FIG. 6 shows on-target editing rates of sDA-BE-TT at the HEK Site 4 target site, including C-to-T editing, C-to-R editing, and indel formation.

FIG. 7 shows on-target editing rates of sDA-BE-TT at the PDCD1 target site, including C-to-T editing, C-to-R editing, and indel formation.

FIG. 8 shows on-target editing rates of sDA-BE-TT at the PPP1R12C 1 target site, including C-to-T editing, C-to-R editing, and indel formation.

FIG. 9 shows on-target editing rates of sDA-BE-TT at the PPP1R12C 2 target site, including C-to-T editing, C-to-R editing, and indel formation.

FIG. 10 shows on-target editing rates of sDA-BE-TT at the PPP1R12C 3 target site, including C-to-T editing, C-to-R editing, and indel formation.

FIG. 11 shows on-target editing rates of sDA-BE-TT at the RNF2 target site, including C-to-T editing, C-to-R editing, and indel formation.

FIG. 12 shows on-target editing rates of sDA-BE-TT at the VEGFA target site, including C-to-T editing, C-to-R editing, and indel formation.

FIG. 13 shows on-target editing rates of sDA-BE-TTER at the EMX1-1 target site, including C-to-T editing, C-to-R editing, and indel formation.

FIG. 14 shows on-target editing rates of sDA-BE-TTER at the EMX1-2 target site, including C-to-T editing, C-to-R editing, and indel formation.

FIG. 15 shows on-target editing rates of sDA-BE-TTER at the FANCF target site, including C-to-T editing, C-to-R editing, and indel formation.

FIG. 16 shows on-target editing rates of sDA-BE-TTER at the HEK Site 2 target site, including C-to-T editing, C-to-R editing, and indel formation.

FIG. 17 shows on-target editing rates of sDA-BE-TTER at the HEK Site 3 target site, including C-to-T editing, C-to-R editing, and indel formation.

FIG. 18 shows on-target editing rates of sDA-BE-TTER at the HEK Site 4 target site, including C-to-T editing, C-to-R editing, and indel formation.

FIG. 19 shows on-target editing rates of sDA-BE-TTER at the PDCD1 target site, including C-to-T editing, C-to-R editing, and indel formation.

FIG. 20 shows on-target editing rates of sDA-BE-TTER at the PPP1R12C 1 target site, including C-to-T editing, C-to-R editing, and indel formation.

FIG. 21 shows on-target editing rates of sDA-BE-TTER at the PPP1R12C 2 target site, including C-to-T editing, C-to-R editing, and indel formation.

FIG. 22 shows on-target editing rates of sDA-BE-TTER at the PPP1R12C 3 target site, including C-to-T editing, C-to-R editing, and indel formation.

FIG. 23 shows on-target editing rates of sDA-BE-TTER at the RNF2 target site, including C-to-T editing, C-to-R editing, and indel formation.

FIG. 24 shows on-target editing rates of sDA-BE-TTER at the VEGFA target site, including C-to-T editing, C-to-R editing, and indel formation.

FIG. 25 shows on-target editing rates of sDA-BE-TTER K34Q at the EMX1-1 target site, including C-to-T editing, C-to-R editing, and indel formation.

FIG. 26 shows on-target editing rates of sDA-BE-TTER K34Q at the EMX1-2 target site, including C-to-T editing, C-to-R editing, and indel formation.

FIG. 27 shows on-target editing rates of sDA-BE-TTER K34Q at the FANCF target site, including C-to-T editing, C-to-R editing, and indel formation.

FIG. 28 shows on-target editing rates of sDA-BE-TTER K34Q at the HEK Site 2 target site, including C-to-T editing, C-to-R editing, and indel formation.

FIG. 29 shows on-target editing rates of sDA-BE-TTER K34Q at the HEK Site 3 target site, including C-to-T editing, C-to-R editing, and indel formation.

FIG. 30 shows on-target editing rates of sDA-BE-TTER K34Q at the HEK Site 4 target site, including C-to-T editing, C-to-R editing, and indel formation.

FIG. 31 shows on-target editing rates of sDA-BE-TTER K34Q at the PDCD1 target site, including C-to-T editing, C-to-R editing, and indel formation.

FIG. 32 shows on-target editing rates of sDA-BE-TTER K34Q at the PPP1R12C 1 target site, including C-to-T editing, C-to-R editing, and indel formation.

FIG. 33 shows on-target editing rates of sDA-BE-TTER K34Q at the PPP1R12C 2 target site, including C-to-T editing, C-to-R editing, and indel formation.

FIG. 34 shows on-target editing rates of sDA-BE-TTER K34Q at the PPP1R12C 3 target site, including C-to-T editing, C-to-R editing, and indel formation.

FIG. 35 shows on-target editing rates of sDA-BE-TTER K34Q at the RNF2 target site, including C-to-T editing, C-to-R editing, and indel formation.

FIG. 36 shows on-target editing rates of sDA-BE-TTER K34Q at the VEGFA target site, including C-to-T editing, C-to-R editing, and indel formation.

FIG. 37 shows on-target editing rates of sDA-BE-TTER K229E compared to sDA-BE-TTER S83R at the HEK Site 4 target site, including C-to-T editing, C-to-R editing, and indel formation.

FIG. 38 shows on-target editing rates of sDA-BE-TTER K229D compared to sDA-BE-TTER S83R at the HEK Site 4 target site, including C-to-T editing, C-to-R editing, and indel formation.

FIG. 39 shows on- and off-target editing rates of sDA-BE-TTER S83R/K229E compared to sDA-BE-TTER S83R at the HEK Site 4 target site, including C-to-T editing, C-to-R editing, and indel formation.

FIG. 40 shows on- and off-target editing rates of sDA-BE-TTER E68C at the HEK Site 4 target site, including C-to-T editing, C-to-R editing, and indel formation.

FIG. 41 shows on- and off-target editing rates of sDA-BE-TTER R33Y at the HEK Site 4 target site, including C-to-T editing, C-to-R editing, and indel formation.

FIG. 42 shows on- and off-target editing rates of sDA-BE-TTER E68W at the HEK Site 4 target site, including C-to-T editing, C-to-R editing, and indel formation.

FIG. 43 shows on- and off-target editing rates of sDA-BE-TTER E68D at the HEK Site 4 target site, including C-to-T editing, C-to-R editing, and indel formation.

FIG. 44 shows on- and off-target editing rates of sDA-BE-TTER E68R at the HEK Site 4 target site, including C-to-T editing, C-to-R editing, and indel formation.

FIG. 45 shows on- and off-target editing rates of sDA-BE-TTER E68K at the HEK Site 4 target site, including C-to-T editing, C-to-R editing, and indel formation.

FIG. 46 shows on- and off-target editing rates of sDA-BE-TTER E68Q at the HEK Site 4 target site, including C-to-T editing, C-to-R editing, and indel formation.

FIG. 47 shows on- and off-target editing rates of sDA-BE-TTER E68H at the HEK Site 4 target site, including C-to-T editing, C-to-R editing, and indel formation.

FIG. 48 shows on- and off-target editing rates of sDA-BE-TTER K34H at the HEK Site 4 target site, including C-to-T editing, C-to-R editing, and indel formation.

FIG. 49 shows on- and off-target editing rates of sDA-BE-TTER R33K at the HEK Site 4 target site, including C-to-T editing, C-to-R editing, and indel formation.

FIG. 50 shows on- and off-target editing rates of sDA-BE-TTER R33Q at the HEK Site 4 target site, including C-to-T editing, C-to-R editing, and indel formation.

FIG. 51 shows on- and off-target editing rates of sDA-BE-TTER R33F at the HEK Site 4 target site, including C-to-T editing, C-to-R editing, and indel formation.

FIG. 52 shows the effect of an extra bipartite NLS on sDA-BE-TTER on-target editing rates.

FIG. 53 shows a comparison of RNA editing rates of sDA-BE-TT and BE3. Comparative cytosine-to-uracil editing rates at six transcripts in HEK293T cells between sDA-BE-TT, BE3, and a dCas9 control. Each data point shows the editing rate of a given known RNA off-target site as edited by BE3 (on the x-axis) and the indicated editor (on the y-axis). As determined by calculating the relative areas under the regression lines shown, sDA-BE-TT exhibits 1.96% of the spurious RNA editing capacity of BE3 across the range of editing shown.

FIG. 54 shows a comparison of RNA editing rates of sDA-BE-TTER and BE3. Comparative cytosine-to-uracil editing rates at six transcripts in HEK293T cells between sDA-BE-TTER, BE3, and a dCas9 control. Each data point shows the editing rate of a given known RNA off-target site as edited by BE3 (on the x-axis) and the indicated editor (on the y-axis). As determined by calculating the relative areas under the regression lines shown, sDA-BE-TTER exhibits 3.36% of the spurious RNA editing capacity of BE3 across the range of editing shown.

FIG. 55 shows a comparison of RNA editing rates of SECURE and BE3. Comparative cytosine-to-uracil editing rates at six transcripts in HEK293T cells between SECURE, BE3, and a dCas9 control. Each data point shows the editing rate of a given known RNA off-target site as edited by BE3 (on the x-axis) and the indicated editor (on the y-axis). As determined by calculating the relative areas under the regression lines shown, SECURE exhibits 22.54% of the spurious RNA editing capacity of BE3 across the range of editing shown.

FIG. 56 shows comparative BE-ARD (spurious DNA) editing of S. aureus sDA-BE-TT and S. aureus SaBE4 at two target sites. Comparative cytosine-to-thymine editing rates at two BE-ARD sites in HEK293T cells between S. aureus sDA-BE-TTER, S. Aureus BE4, and a sDA 1.2 only control. As determined by calculating the relative areas under the regression lines shown, sDA-BE-TTER exhibits 10.61% of the spurious DNA editing capacity of BE3 across the range of editing shown.

FIG. 57 shows two examples of a dual-targeting sDA-BE, comprising one fusion protein of a KKH dSaCas9 molecule fused an sDA1 domain and an nCas9-sDA2-UGI-UGI fusion. Both “Pro” and “Anti” orientations are shown, with the Cas9 molecules targeting the same direction or opposite directions, respectively.

FIG. 58 shows a summary of both on-target and BE-ARD experiments of our dual-targeting sDA-BE constructs. The magnitude of the y-axis shows the ratio of enhancement over standard (higher values mean a more favorable on-target editing:spurious editing compared to a matched control lacking an on-target dSaCas9 target site). nSpCas9 target sites are indicated, with the dSaCas9 target sites described in terms of their relative position to the nSpCas9 sites and their orientation.

FIGS. 59A-59C show graphical representations of BE4Max and sDA-BEs. FIG. 59A shows a graphical representation of BE4Max. FIG. 59B shows a graphical representation of an embodiment of an sDA-BE. FIG. 59C shows a graphical representation of an embodiment of an sDA-BE, with an extra bipartite nuclear localization signal (NLS).

FIG. 60 shows a predicted structure of the rAPO1 domain, with the sDA pieces annotated, as well as the R33 and K34 residues (Grunewald et al., “Transcriptome-wide off-target RNA editing induced by CRISPR-guided DNA base editors,” Nature 569(7756):433-437 (2019)).

FIG. 61 shows a schematic representation of a number of possible sDA-BE architectures in terms of their domain substructures in comparison to an intact BE (BE4Max).

FIG. 62 shows a heatmap chart plotting the on-target cytosine editing of the of sDA-BEs described in FIG. 63 in comparison to various intact editors, including BE4Max as well as mutant versions of it as indicated, at 12 gRNA target sites. Cytosine target positions are shown and referred to by their position in the gRNA target sequence. Full opacity corresponds to 100% cytosine editing in this plot.

FIG. 63 shows a graphical representation of the BE-ARD assay, with either an intact BE (as in BE4Max) (left) or an sDA-BE (right).

FIG. 64 shows a heatmap chart plotting the spurious DNA editing as determined by the BE-ARD assay of the of sDA-BEs described in FIG. 63 in comparison to various intact editors, including BE4Max as well as mutant versions of it as indicated, at 5 SaCas9 gRNA target sites. Cytosine target positions are shown and referred to by their position in the gRNA target sequence. Full opacity corresponds to 30% cytosine editing in this plot.

FIG. 65 shows a summary of the normalized total cytosine editing (“C-to-D editing”) compared to BE4Max of a given BE across all on-target sites examined in this figure.

FIG. 66 shows a summary of the normalized total cytosine editing (“C-to-D editing”) compared to BE4Max of a given BE across all SaCas9 BE-ARD sites examined in this figure.

FIG. 67 shows a graphical representation of an experiment in which a titration series of BE4Max is used as a standard to determine the relative deaminase concentration experience by a population of cells at a given delivery amount of sDA-BE. The heatmap plot shows on-target editing of 160,000 HEK293T cells transfected with the indicated amount of plasmid encoding the indicated BE. Full opacity corresponds to 100% cytosine editing in this plot.

FIG. 68 summarizes the relative deaminase concentration experienced at each type of site across all experiments, normalized to BE4Max. Six titration comparison experiments were performed as described in FIG. 67, three of which were conducted at on-target sites and three at BE-ARD sites. We show that on-target sites consistently experience a higher concentration of deaminase compared to spurious BE-ARD sites, thus suggesting the molecularity effect may explain the differential editing deficit experienced at spurious DNA sites compared to on-target sites.

FIG. 69 shows a graphical representation of the molecularity effect. With an intact editor, on-target sites can be modeled as a “uni-molecular” reaction due to the occupancy time of Cas9 (Sternberg et al., “DNA interrogation by the CRISPR RNA-guided endonuclease Cas9.” Nature 507(7490):62-67 (2014)) whereas a spurious DNA editing event is a bi-molecular reaction. An sDA-BE editor can be modelled as a bi-molecular reaction at an on-target site, since an nCas9-sDA2 molecule should accumulate there and can wait for an interaction with an untethered sDA1 molecule from solution. By contrast, a spurious DNA editing event requires the collision of all three molecules in a ter-molecular reaction, which is relatively unlikely compared to uni-molecular or bi-molecular reactions.

FIG. 70 is a heat-map showing triplicate on-target Cytosine-to-Adenine editing rates of a sDA-BE-TTER (−UGI) compared to BE4-Max, sDA-BE-TTER (+NLS), and a matched nCas9-UGI-UGI (nUGI) control at the EMX1 Site 1 target site. Editing is shown at gRNA cytosines C5, C6, and C10 (X-axis), with maximum C-to-A editing of 8.36%.

FIG. 71 is a heat-map showing triplicate on-target Cytosine-to-Adenine editing rates of a sDA-BE-TTER (−UGI) compared to BE4-Max, sDA-BE-TTER (+NLS), and a matched nCas9-UGI-UGI (nUGI) control at the EMX1 Site 2 target site. Editing is shown at gRNA cytosines C6, C8, and C9 (X-axis), with maximum C-to-A editing of 8.14%.

FIG. 72 is a heat-map showing triplicate on-target Cytosine-to-Adenine editing rates of a sDA-BE-TTER (−UGI) compared to BE4-Max, sDA-BE-TTER (+NLS), and a matched nCas9-UGI-UGI (nUGI) control at the FANCF target site. Editing is shown at gRNA cytosines C6, C7, C8, and C11 (X-axis), with maximum C-to-A editing of 2.54%.

FIG. 73 is a heat-map showing triplicate on-target Cytosine-to-Adenine editing rates of a sDA-BE-TTER (−UGI) compared to BE4-Max, sDA-BE-TTER (+NLS), and a matched nCas9-UGI-UGI (nUGI) control at the HEK Site 2 target site. Editing is shown at gRNA cytosines C4, C6, and C11 (X-axis), with maximum C-to-A editing of 4.81%.

FIG. 74 is a heat-map showing triplicate on-target Cytosine-to-Adenine editing rates of a sDA-BE-TTER (−UGI) compared to BE4-Max, sDA-BE-TTER (+NLS), and a matched nCas9-UGI-UGI (nUGI) control at the HEK Site 3 target site. Editing is shown at gRNA cytosines C3, C4, C5, and C9 (X-axis), with maximum C-to-A editing of 3.58%.

FIG. 75 is a heat-map showing triplicate on-target Cytosine-to-Adenine editing rates of a sDA-BE-TTER (−UGI) compared to BE4-Max, sDA-BE-TTER (+NLS), and a matched nCas9-UGI-UGI (nUGI) control at the HEK Site 4 target site. Editing is shown at gRNA cytosines C3, C5, C8, and C11 (X-axis), with maximum C-to-A editing of 12.68%.

FIG. 76 is a heat-map showing triplicate on-target Cytosine-to-Adenine editing rates of a sDA-BE-TTER (−UGI) compared to BE4-Max, sDA-BE-TTER (+NLS), and a matched nCas9-UGI-UGI (nUGI) control at the PDCD1 target site. Editing is shown at gRNA cytosines C6, C9, C10, and C12 (X-axis), with maximum C-to-A editing of 16.93%.

FIG. 77 is a heat-map showing triplicate on-target Cytosine-to-Adenine editing rates of a sDA-BE-TTER (−UGI) compared to BE4-Max, sDA-BE-TTER (+NLS), and a matched nCas9-UGI-UGI (nUGI) control at the PPP1R12C site 1 target site. Editing is shown at gRNA cytosines C3, C5, C7, C8 and C9 (X-axis), with maximum C-to-A editing of 8.96%.

FIG. 78 is a heat-map showing triplicate on-target Cytosine-to-Adenine editing rates of a sDA-BE-TTER (−UGI) compared to BE4-Max, sDA-BE-TTER (+NLS), and a matched nCas9-UGI-UGI (nUGI) control at the PPP1R12C site 2 target site. Editing is shown at gRNA cytosines C3, C5, and C7 (X-axis), with maximum C-to-A editing of 10.64%.

FIG. 79 is a heat-map showing triplicate on-target Cytosine-to-Adenine editing rates of a sDA-BE-TTER (−UGI) compared to BE4-Max, sDA-BE-TTER (+NLS), and a matched nCas9-UGI-UGI (nUGI) control at the PPP1R12C site 3 target site. Editing is shown at gRNA cytosines C4, C6, and C8 (X-axis), with maximum C-to-A editing of 11.06%.

FIG. 80 is a heat-map showing triplicate on-target Cytosine-to-Adenine editing rates of a sDA-BE-TTER (−UGI) compared to BE4-Max, sDA-BE-TTER (+NLS), and a matched nCas9-UGI-UGI (nUGI) control at the RNF2 target site. Editing is shown at gRNA cytosines C3, C6, and C12 (X-axis), with maximum C-to-A editing of 6.87%.

FIG. 81 is a heat-map showing triplicate on-target Cytosine-to-Adenine editing rates of a sDA-BE-TTER (−UGI) compared to BE4-Max, sDA-BE-TTER (+NLS), and a matched nCas9-UGI-UGI (nUGI) control at the VEGFA target site. Editing is shown at gRNA cytosines C3, C4, C5, C6, C7, C9, C10, and C12 (X-axis), with maximum C-to-A editing of 14.15%.

FIG. 82 is a heat-map showing triplicate on-target Cytosine-to-Guanine editing rates of a sDA-BE-TTER (−UGI) compared to BE4-Max, sDA-BE-TTER (+NLS), and a matched nCas9-UGI-UGI (nUGI) control at the EMX1 Site 1 target site. Editing is shown at gRNA cytosines C5, C6, and C10 (X-axis), with maximum C-to-G editing of 9.66%.

FIG. 83 is a heat-map showing triplicate on-target Cytosine-to-Guanine editing rates of a sDA-BE-TTER (−UGI) compared to BE4-Max, sDA-BE-TTER (+NLS), and a matched nCas9-UGI-UGI (nUGI) control at the EMX1 Site 2 target site. Editing is shown at gRNA cytosines C6, C8, and C9 (X-axis), with maximum C-to-G editing of 1.59%.

FIG. 84 is a heat-map showing triplicate on-target Cytosine-to-Guanine editing rates of a sDA-BE-TTER (−UGI) compared to BE4-Max, sDA-BE-TTER (+NLS), and a matched nCas9-UGI-UGI (nUGI) control at the FANCF target site. Editing is shown at gRNA cytosines C6, C7, C8, and C11 (X-axis), with maximum C-to-G editing of 1.35%.

FIG. 85 is a heat-map showing triplicate on-target Cytosine-to-Guanine editing rates of a sDA-BE-TTER (−UGI) compared to BE4-Max, sDA-BE-TTER (+NLS), and a matched nCas9-UGI-UGI (nUGI) control at the HEK Site 2 target site. Editing is shown at gRNA cytosines C4, C6, and C11 (X-axis), with maximum C-to-G editing of 60.44%.

FIG. 86 is a heat-map showing triplicate on-target Cytosine-to-Guanine editing rates of a sDA-BE-TTER (−UGI) compared to BE4-Max, sDA-BE-TTER (+NLS), and a matched nCas9-UGI-UGI (nUGI) control at the HEK Site 3 target site. Editing is shown at gRNA cytosines C3, C4, C5, and C9 (X-axis), with maximum C-to-G editing of 17.46%.

FIG. 87 is a heat-map showing triplicate on-target Cytosine-to-Guanine editing rates of a sDA-BE-TTER (−UGI) compared to BE4-Max, sDA-BE-TTER (+NLS), and a matched nCas9-UGI-UGI (nUGI) control at the HEK Site 4 target site. Editing is shown at gRNA cytosines C3, C5, C8, and C11 (X-axis), with maximum C-to-G editing of 29.62%.

FIG. 88 is a heat-map showing triplicate on-target Cytosine-to-Guanine editing rates of a sDA-BE-TTER (−UGI) compared to BE4-Max, sDA-BE-TTER (+NLS), and a matched nCas9-UGI-UGI (nUGI) control at the PDCD1 target site. Editing is shown at gRNA cytosines C6, C9, C10, and C12 (X-axis), with maximum C-to-G editing of 16.87%.

FIG. 89 is a heat-map showing triplicate on-target Cytosine-to-Guanine editing rates of a sDA-BE-TTER (−UGI) compared to BE4-Max, sDA-BE-TTER (+NLS), and a matched nCas9-UGI-UGI (nUGI) control at the PPP1R12C site 1 target site. Editing is shown at gRNA cytosines C3, C5, C7, C8 and C9 (X-axis), with maximum C-to-G editing of 1.37%.

FIG. 90 is a heat-map showing triplicate on-target Cytosine-to-Guanine editing rates of a sDA-BE-TTER (−UGI) compared to BE4-Max, sDA-BE-TTER (+NLS), and a matched nCas9-UGI-UGI (nUGI) control at the PPP1R12C site 2 target site. Editing is shown at gRNA cytosines C3, C5, and C7 (X-axis), with maximum C-to-G editing of 3.02%.

FIG. 91 is a heat-map showing triplicate on-target Cytosine-to-Guanine editing rates of a sDA-BE-TTER (−UGI) compared to BE4-Max, sDA-BE-TTER (+NLS), and a matched nCas9-UGI-UGI (nUGI) control at the PPP1R12C site 3 target site. Editing is shown at gRNA cytosines C4, C6, and C8 (X-axis), with maximum C-to-G editing of 16.70%.

FIG. 92 is a heat-map showing triplicate on-target Cytosine-to-Guanine editing rates of a sDA-BE-TTER (−UGI) compared to BE4-Max, sDA-BE-TTER (+NLS), and a matched nCas9-UGI-UGI (nUGI) control at the RNF2 target site. Editing is shown at gRNA cytosines C3, C6, and C12 (X-axis), with maximum C-to-G editing of 32.20%.

FIG. 93 is a heat-map showing triplicate on-target Cytosine-to-Guanine editing rates of a sDA-BE-TTER (−UGI) compared to BE4-Max, sDA-BE-TTER (+NLS), and a matched nCas9-UGI-UGI (nUGI) control at the VEGFA target site. Editing is shown at gRNA cytosines C3, C4, C5, C6, C7, C9, C10, and C12 (X-axis), with maximum C-to-G editing of 2.88%.

FIG. 94 is a heat-map showing triplicate on-target Cytosine-to-Thymine editing rates of a sDA-BE-TTER (−UGI) compared to BE4-Max, sDA-BE-TTER (+NLS), and a matched nCas9-UGI-UGI (nUGI) control at the EMX Site 1 target site. Editing is shown at gRNA cytosines C5, C6, and C10 (X-axis), with maximum C-to-T editing of 44.54%.

FIG. 95 is a heat-map showing triplicate on-target Cytosine-to-Thymine editing rates of a sDA-BE-TTER (−UGI) compared to BE4-Max, sDA-BE-TTER (+NLS), and a matched nCas9-UGI-UGI (nUGI) control at the EMX Site 2 target site. Editing is shown at gRNA cytosines C6, C8, and C9 (X-axis), with maximum C-to-T editing of 26.78%.

FIG. 96 is a heat-map showing triplicate on-target Cytosine-to-Thymine editing rates of a sDA-BE-TTER (−UGI) compared to BE4-Max, sDA-BE-TTER (+NLS), and a matched nCas9-UGI-UGI (nUGI) control at the FANCF target site. Editing is shown at gRNA cytosines C6, C7, C8, and C11 (X-axis), with maximum C-to-T editing of 31.67%.

FIG. 97 is a heat-map showing triplicate on-target Cytosine-to-Thymine editing rates of a sDA-BE-TTER (−UGI) compared to BE4-Max, sDA-BE-TTER (+NLS), and a matched nCas9-UGI-UGI (nUGI) control at the HEK Site 2 target site. Editing is shown at gRNA cytosines C4, C6, and C11 (X-axis), with maximum C-to-T editing of 82.74%.

FIG. 98 is a heat-map showing triplicate on-target Cytosine-to-Thymine editing rates of a sDA-BE-TTER (−UGI) compared to BE4-Max, sDA-BE-TTER (+NLS), and a matched nCas9-UGI-UGI (nUGI) control at the HEK Site 3 target site. Editing is shown at gRNA cytosines C3, C4, C5, and C9 (X-axis), with maximum C-to-T editing of 75.63%.

FIG. 99 is a heat-map showing triplicate on-target Cytosine-to-Thymine editing rates of a sDA-BE-TTER (−UGI) compared to BE4-Max, sDA-BE-TTER (+NLS), and a matched nCas9-UGI-UGI (nUGI) control at the HEK Site 4 target site. Editing is shown at gRNA cytosines C3, C5, C8, and C11 (X-axis), with maximum C-to-T editing of 64.96%.

FIG. 100 is a heat-map showing triplicate on-target Cytosine-to-Thymine editing rates of a sDA-BE-TTER (−UGI) compared to BE4-Max, sDA-BE-TTER (+NLS), and a matched nCas9-UGI-UGI (nUGI) control at the PDCD1 target site. Editing is shown at gRNA cytosines C6, C9, C10, and C12 (X-axis), with maximum C-to-T editing of 53.8%.

FIG. 101 is a heat-map showing triplicate on-target Cytosine-to-Thymine editing rates of a sDA-BE-TTER (−UGI) compared to BE4-Max, sDA-BE-TTER (+NLS), and a matched nCas9-UGI-UGI (nUGI) control at the PPP1R12C site 1 target site. Editing is shown at gRNA cytosines C3, C5, C7, C8 and C9 (X-axis), with maximum C-to-T editing of 60.54%.

FIG. 102 is a heat-map showing triplicate on-target Cytosine-to-Thymine editing rates of a sDA-BE-TTER (−UGI) compared to BE4-Max, sDA-BE-TTER (+NLS), and a matched nCas9-UGI-UGI (nUGI) control at the PPP1R12C site 2 target site. Editing is shown at gRNA cytosines C3, C5, and C7 (X-axis), with maximum C-to-T editing of 77.74%.

FIG. 103 is a heat-map showing triplicate on-target Cytosine-to-Thymine editing rates of a sDA-BE-TTER (−UGI) compared to BE4-Max, sDA-BE-TTER (+NLS), and a matched nCas9-UGI-UGI (nUGI) control at the PPP1R12C site 3 target site. Editing is shown at gRNA cytosines C4, C6, and C8 (X-axis), with maximum C-to-T editing of 67.31%.

FIG. 104 is a heat-map showing triplicate on-target Cytosine-to-Thymine editing rates of a sDA-BE-TTER (−UGI) compared to BE4-Max, sDA-BE-TTER (+NLS), and a matched nCas9-UGI-UGI (nUGI) control at the RNF2 target site. Editing is shown at gRNA cytosines C3, C6, and C12 (X-axis), with maximum C-to-T editing of 58.77%.

FIG. 105 is a heat-map showing triplicate on-target Cytosine-to-Thymine editing rates of a sDA-BE-TTER (−UGI) compared to BE4-Max, sDA-BE-TTER (+NLS), and a matched nCas9-UGI-UGI (nUGI) control at the VEGFA target site. Editing is shown at gRNA cytosines C3, C4, C5, C6, C7, C9, C10, and C12 (X-axis), with maximum C-to-T editing of 99.0%.

FIG. 106 shows the nucleotide sequence (5′→3′, SEQ ID NO:70; 3′ 4 5′, SEQ ID NO:71) and amino acid (SEQ ID NO:72) sequences and structure of plasmid pCMV_BE4max (addgene number 112093) (Improving cytidine and adenine base editors by expression optimization and ancestral reconstruction. Koblan L W, Doman J L, Wilson C, Levy J M, Tay T, Newby G A, Maianti J P, Raguram A, Liu D R. Nat Biotechnol. 2018 May 29. pii: nbt.4172. doi: 10.1038/nbt.4172. 10.1038/nbt.4172 PubMed 29813047) carrying the full length BE4Max sequence (nucleotides 409-5967). The nucleotides encoding the bi-partite NLSs are located at bp 409-465 and 5917-5967). The nucleotides encoding APOBEC-1 are located at bp 466-1149). The nucleotides encoding Cas9(D10A) are located at bp 1246-5346). The nucleotides encoding the UGIs are located at bp 5377-5625 and 5656-5904).

### DETAILED DESCRIPTION

Because of the natural ability of AID/APOBEC cytosine deaminase enzymes to deaminate cytosines in single stranded genomic DNA and RNA, CRISPR Base Editor technology can result in non-specific, spurious deamination events leading to off-target mutagenesis in the transcriptome and regions of the genome that are exposed as ssDNA, such as actively transcribed regions or DNA undergoing replication. In fact, an E. coli-based assay examining deaminases showed that an actively transcribed region of the genome could be highly enriched (˜7-530 fold) for C→T transition mutations when exposed to various overexpressed mammalian deaminases. See Harris et al., “RNA Editing Enzyme APOBEC1 and Some of Its Homologs Can Act as DNA Mutators.” Molecular Cell 10.5 (2002): 1247-253. Further, co-expression of the cytosine deaminase PmCda1 and nCas9 as two separate, untethered proteins in yeast cells results in similar levels of deamination at the sgRNA-specified target site as when the two components are expressed as direct fusion partners, demonstrating that these proteins are capable of deaminating ssDNA from solution without an affinity tether to the genomic location. See Nishida et al., “Targeted Nucleotide Editing Using Hybrid Prokaryotic and Vertebrate Adaptive Immune Systems.” Science 353.6305 (2016). This concern is especially relevant now that scientists are becoming increasingly aware that R-loops are a more common occurrence in the genomes of eukaryotic cells than previously thought, thus creating many potential steady-state off-target ssDNA substrates where an APOBEC could bind and deaminate. See Santos-Pereira, Jose M., and Andres Aguilera. “R Loops: New Modulators of Genome Dynamics and Function.” Nature Reviews Genetics 16.10 (2015): 583-97. While it is as yet unproven whether BE overexpression itself can sufficiently stimulate spurious deamination and mutagenesis on a global genomic scale, aberrant and over-active APOBEC deaminase activity is a known driver of tumorigenic mutagenesis (see Rebhandl et al. “AID/APOBEC Deaminases and Cancer.” Oncoscience 2 (2015): 320) and overexpression of hAPO3 (see Suspene et al., “Recovery of APOBEC3-edited human immunodeficiency virus G→A hypermutants by differential DNA denaturation PCR.” Journal of General Virology 86.1 (2005): 125-129; Aynaud et al., “Human Tribbles 3 protects nuclear DNA from cytidine deamination by APOBEC3A.” Journal of Biological Chemistry 287.46 (2012): 39182-39192; Shinohara et al., “APOBEC3B can impair genomic stability by inducing base substitutions in genomic DNA in human cells.” Scientific Reports 2 (2012): 806; and Holtz et al., “APOBEC3G cytosine deamination hotspots are defined by both sequence context and single-stranded DNA secondary structure.” Nucleic Acids Research (2013): gkt246) has been shown to stimulate genomic cytosine hypermutation. Thus, it stands to reason that limiting the naturally global deaminating activity of over-expressed deaminases like BE will be important for translating BE technologies into therapeutic applications. Of note, since BE includes the UGI inhibitor to bias deamination events toward productive C→T mutations, it is possible that global off-target BE activity is even more mutagenic than the effects of aberrant deaminase activity alone during tumorigenesis.

We predicted that split-deaminase base editors (sDA-BEs) would have reduced capacities for inducing spurious editing events by increasing the molecularity of the editing reaction, thereby reducing the chances that transient spurious DNA binding events will lead to productive editing events, whereas the long residence time of Cas9 at the on-target site will have enough time to re-form the enzyme from a lucky collision with its complement piece. Herein we described engineered split deaminase base editors (sDA-BE) with reduced capacity for inducing spurious editing events.

### Split Deaminase Base Editors

Split deaminase base editors are described, e.g., in U.S. Patent Application Publication No. 2020/0172895. The present disclosure is based at least in part on the surprising discovery that engineered BEs that use split deaminases (sDA) are functional even when only one of them is tethered to DNA.

CRISPR Cytosine Base Editors (CBEs) enable precise cytosine-to-thymine genetic mutations via APOBEC-mediated deamination of CRISPR-targeted cytosines, but may possess the ability to induce gRNA-independent DNA edits through the action of their deaminase domain. Here, we report the development of a new class of split-deaminase CBEs (sDA-BEs) that severely limit this important dimension of off-target editing while maintaining a high level of on-target editing activity. Finally, we examine a mechanistic explanation of this outcome, which poses that split-deaminase CBEs create limiting concentrations of active deaminase inside the nucleus generally while maintaining robust concentrations of deaminase at the on-target site through CRISPR-mediated accumulation at that position.

While CRISPR Cytosine Base Editing technologies (CBEs) can create site-specific point mutations in eukaryotic cells, their potential to create genome-wide gRNA-independent DNA edits through the independent action of their deaminase domain has always been a salient concern regarding their prospects as therapeutic agents. Aberrant and over-active APOBEC deaminase activity is a known driver of tumorigenic mutagenesis, and several reports have emerged showing the potential for cytosine BEs (CBEs) to mutate both RNA (˜10⁴-10⁵ edits observed) and gDNA (˜10² edits observed) in an unguided manner. For simplicity, we classify this form of gRNA-independent editing as spurious editing.

Though engineered CBE variants possessing modified rAPOBEC1 (rAPO1) domains can limit spurious DNA editing, such alterations often carry severe on-target editing penalties, especially those that reduce spurious DNA editing to minimal levels. We hypothesized that a split-deaminase CBE would possess a limited capacity to undergo spurious DNA editing by limiting the nuclear concentration of the deaminase enzyme, thus protecting non-targeted DNA from exposure to its editing potential, a mechanism that was first proposed in a report from George Church's group and closely mimics a similar approach to prevent spurious off-target effects of targeted DNA methyltransferases.

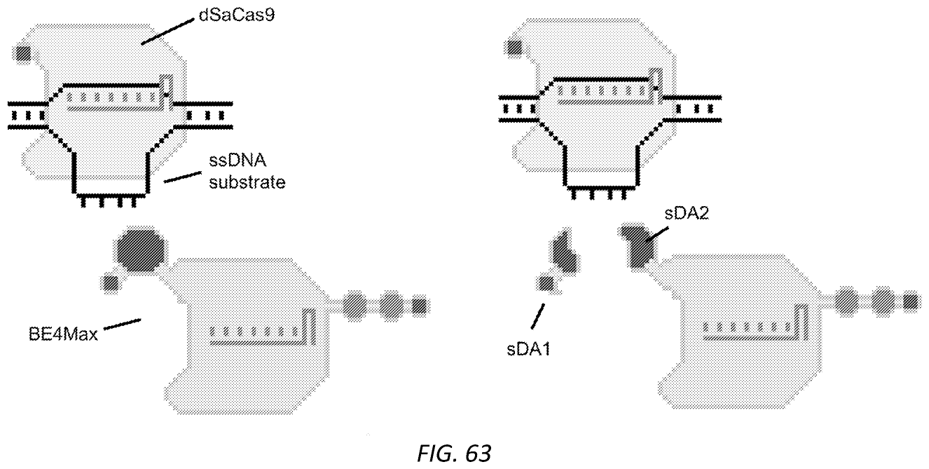

To enable the rapid and robust comparisons between different kinds of CBEs with respect to their capacities to stimulate spurious DNA edits, we used an orthogonal dual Cas9 editing approach capable of determining the relative capacities of CBEs to stimulate unguided C-to-T edits. First, a catalytically-inactivated Cas9 (dCas9) and a gRNA is expressed in cells, which creates a long-lived ssDNA substrate at which a co-expressed orthogonal CBE can act. The cytosine editing rates observed at these sites is then taken as a reflection of the CBE's capacity for spuriously editing DNA. Critically, the dCas9 molecule is targeted to its site via an orthogonal gRNA that does not cross-react with the nCas9 domain of the CBE, which we achieve here by using a dCas9 from Staphylococcus aureus. This assay, which we term Base Editing at Anchored R-Loop DNA (BE-ARD) allows us to conduct an in situ experiment that closely replicates the chemical conditions required for spurious DNA editing, and therefore allows us to make informed assertions about the relative spurious DNA editing capacities of various CBEs. A graphical representation of this assay is shown in FIG. 63.

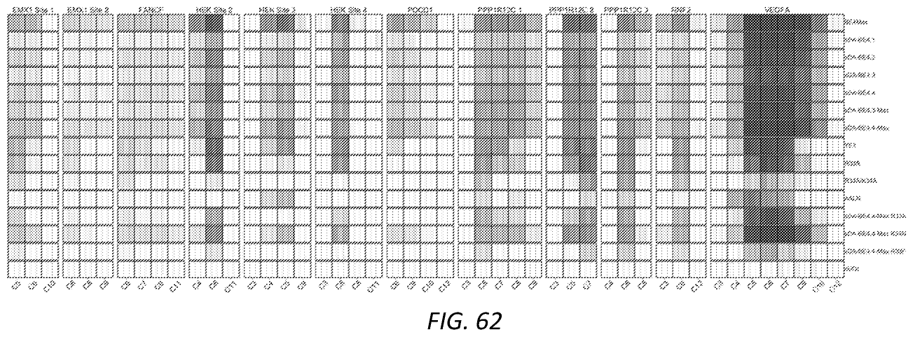

Work from David Liu's lab has established that reduced-activity variant CBEs—namely the YE variants and SECURE variants bearing the R33A and K34A mutations—possess markedly lower capacities for undergoing spurious DNA editing events than wild-type CBEs (Kim et al., “Increasing the genome-targeting scope and precision of base editing with engineered Cas9-cytidine deaminase fusions,” Nat. Biotechnol. 35(4):371-376 (2017); Grunewald et al., “Transcriptome-wide off-target RNA editing induced by CRISPR-guided DNA base editors.” Nature 569(7756):433-437 (2019); and Doman et al., “Evaluation and minimization of Cas9-independent off-target DNA editing by cytosine base editors,” Nat. Biotechnol. 38(5):620-628. (2020)). Our own studies of SECURE editors using the BE-ARD assay confirm this finding; however, we also found that the total on-target editing rates of SECURE CBEs can suffer significantly, with the R33A/K34A double mutant unable to achieve 25% of the total cytosine editing of its parent enzyme (BE4Max) at 8 of the 12 sites we examined. Meanwhile the R33A version failed to achieve 25% of the total editing of its parent enzyme at 2 of 12 sites (FIG. 62). We also examined the YE1 variant, which possesses slightly higher rates of both on-target and spurious DNA editing than the R33A variant.

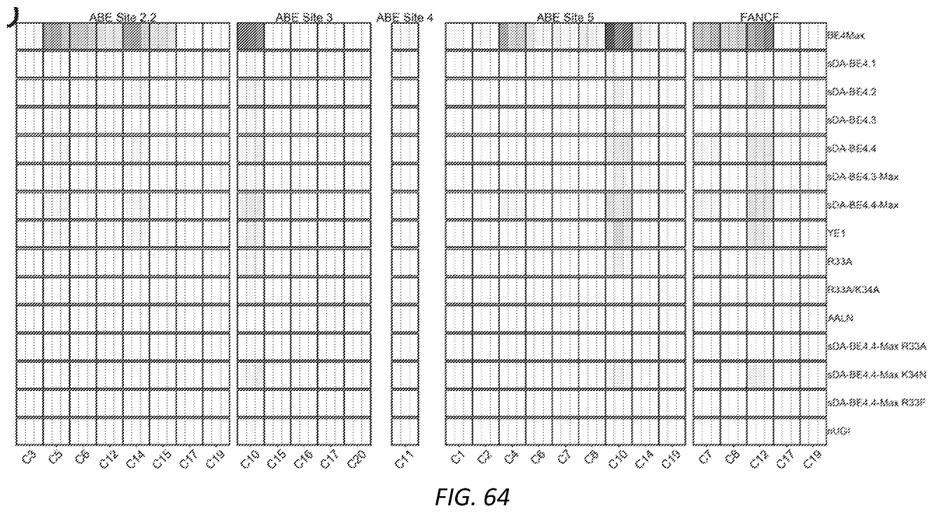

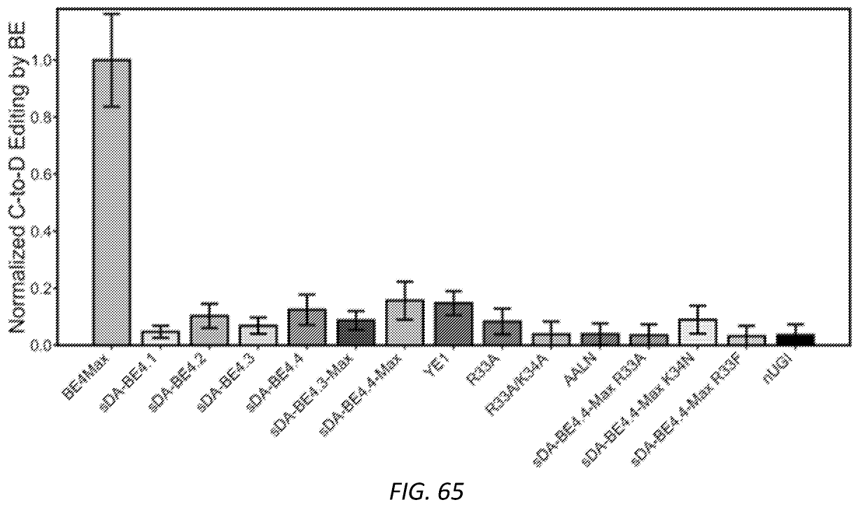

A version of the R33A/K34A SECURE variant bearing the activity-enhancing H140L/D142N mutations (the so-called AALN variant) has been put forth as a partial solution to the activity decrement observed with SECURE CBEs. Previous work proposes that AALN rescues some of the on-target activity of the R33A/K34A variant while maintaining its favorable effects on spurious DNA editing; however, careful examination of published data shows an activity increase at only 1 of 6 gRNA sites tested (Doman et al., “Evaluation and minimization of Cas9-independent off-target DNA editing by cytosine base editors,” Nat. Biotechnol. 38(5):620-628 (2020)). We confirm and extend this finding, showing that total cytosine editing rates with AALN are improved over R33A/K34A at only 2 out of the 12 gRNA sites we examined. These are also the only 2 gRNA sites that reach at least 25% editing using the AALN CBE, although we also note that spurious DNA editing with AALN is indistinguishable from a deaminase-free nCas9-UGI-UGI (nUGI) control at all 5 SaCas9 sites examined in our BE-ARD assay (FIG. 64 and FIG. 65).

Recognizing the need for an alternate approach to abrogate spurious DNA editing while maintaining on-target activity, we next examined an sDA-BE architecture. While we initially envisioned a system in which two cognate subdomains of an sDA would be co-localized to a loci via adjacently-targeting Cas9 modules, we found that by co-expressing both pieces that result from the splitting of BE4Max between the T72 and P78 amino acid residues of its rAPO1 domain (T90 and P96 with respect to the BE4Max sequence) was able to modestly reproduce the cytosine editing rates of its parent enzyme at 12 on-target sites in HEK293T cells (sDA-BE4.1). We chose this split region due to its lack of secondary structure as observed in a predicted structural representation and the fact that it bisects the active site of the enzyme, making each piece unlikely to be active on its own, which we then confirmed. For simplicity, we refer to the smaller N-terminal piece as sDA1, and the nCas9-containing C-terminal piece as sDA2. Importantly, sDA-BE4.1 also largely attenuates spurious DNA editing as observed in the BE-ARD assay, with editing rates at 5 SaCas9 sites nearly identical to a nUGI control.

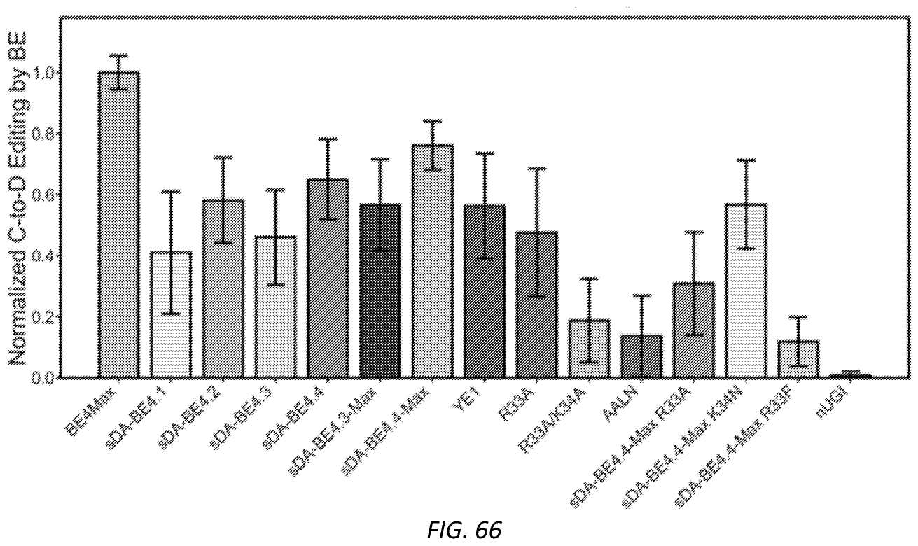

We also reasoned that inclusion of charged residues on the termini of the sDA pieces may improve protein stability through solvent effects, and thus found that an extension of the N-terminal end of the C-terminal domain to include E73 and R74 (ER extension) also improves editing activity (sDA-BE4.2). Next, we visually scanned a predicted rAPO1 structure for residues that could plausibly influence the binding affinity between the N-terminal and C-terminal sDA pieces. This led to our discovery that an incorporation of an S83R mutation improves on target editing rates (sDA-BE4.3). An sDA-BE containing both the S83R and ER modifications exhibits higher editing rates than both (sDA-BE4.4). Finally, inclusion of a second bi-partite NLS domain in between the UGI domains of the C-terminal sDA-containing protein increases on-target editing rates of both sDA-BE4.3 and sDA-BE4.4 (sDA-BE4.3-Max and sDA-BE4.4-Max). Importantly, sDA-BE4.4-Max exhibits on-target activity above 75% of BE4Max at all gRNAs examined and possesses reduced rates of spurious DNA editing in the BE-ARD assay, with less than 20% of the total spurious editing as BE4Max across all sites (FIG. 62, FIG. 64, FIG. 65 and FIG. 66). Table 6 contains information pertaining to the sequences comprising each of the sDA-BEs described in this paragraph.

We next sought to combine the sDA-BE and SECURE strategies to achieve a total abrogation of spurious DNA editing. Because the R33A/K34A double mutant has serious deficiencies in editing some on-target sites, we focused on a version of sDA-BE4.4-Max bearing the R33A mutation. Unsurprisingly, this sDA-SECURE editor showed undetectable rates of spurious DNA editing at all 5 BE-ARD sites examined; however, it also has difficulty editing the PDCD1 and HEK Site 3 gRNA sites that its parent enzyme (BE4Max R33A) similarly struggles to edit, suggesting that its utility may need to be empirically investigated by users on a case-by-case basis (FIG. 62, FIG. 64, FIG. 65 and FIG. 66). To examine the effects of similar mutations, we also created two additional sDA-BE4.4-Max variants bearing the SECURE-like mutants K34N or R33F mutations, revealing either intermediate effects on editing efficiency and specificity (K34N) or severe effects (R33F) (FIG. 62, FIG. 64, FIG. 65 and FIG. 66).

We realized that one potential explanation for why sDA-BEs reduce spurious DNA editing may be that they result in a diminished nuclear concentration of enzymatically active deaminase domain compared to an equivalent molar amount of intact CBE. That is, that the two sDA pieces only transiently reform themselves into a functional deaminase domain. While this scenario may account for some of the effects we observe, if it was the only mechanism underlying the spurious-limiting effects of sDA-BEs, our outcomes could be achieved by simply delivering a lower dose of an intact CBE.

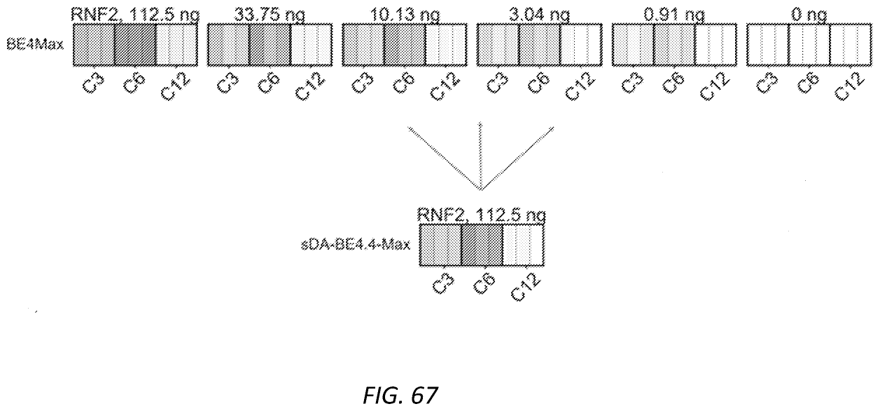

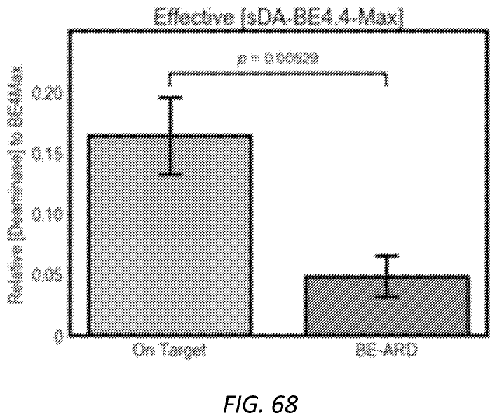

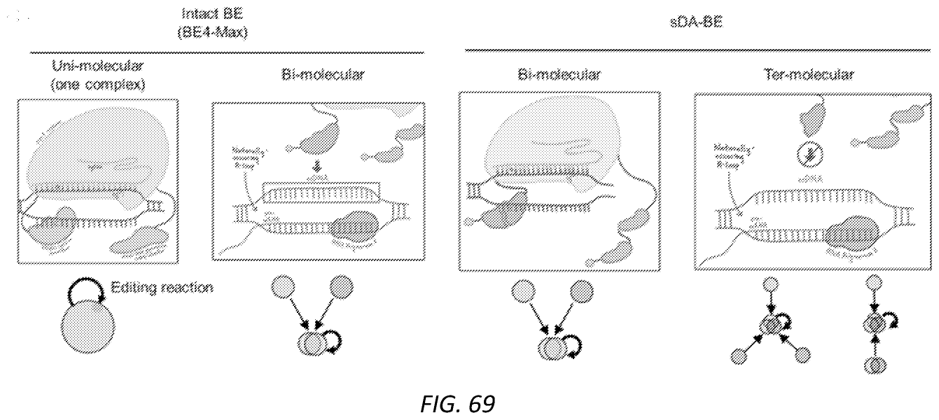

To investigate this possibility, we transfected HEK293T cells with a 30% dilution series of either BE4Max starting at 112.5 ng and three separate on-target and BE-ARD gRNA pairs and plotted the resultant total C-to-D editing rates at both kinds of sites (FIG. 67). By creating standard sigmoidal curves of the resultant editing rates, we were able to estimate the concentration of sDA-BE4.4-Max relative to BE4Max at both kinds of target sites. This analysis revealed that sDA-BE4.4-Max has a relative concentration of ˜0.16 (+/−0.04) at on-target sites compared to BE4Max, while this value drops to ˜0.05 (+/−0.01) at the BE-ARD target sites (FIG. 68). This differential effect suggests that a simple concentration-based explanation is unlikely to explain our findings, since the relative deaminase concentration between on-target and BE-ARD sites should have remained stable at both kinds of sites if nuclear concentration is the only important variable that explains our results. We therefore hypothesize that sDA-BEs are able to differentially reduce spurious DNA editing via a molecularity effect, wherein the nCas9-mediated accumulation of one piece of the sDA allows its deamination reaction to proceed as a functionally bi-molecular reaction, whereas editing at a spurious DNA site would require a relatively rare ter-molecular collision between both sDA pieces and an ssDNA substrate (FIG. 69).

The development of an sDA-BE architecture is an important step toward creating maximally-specific CBEs, and may therefore be a critical step toward routine therapeutic adoption of this technology. Though this study relied solely on the highly-sensitive in situ BE-ARD approach to compare the spurious DNA editing capacities between various CBEs, previous studies have shown that CBE treatments are capable of producing thousands of genomically-dispersed spurious DNA editing events. Though this level of unpredictable off-target editing may be acceptable for some industrial or experimental therapeutic applications, it may prevent the widespread adoption of CBEs for routine therapeutic uses where disease prognoses are not severe enough to warrant the oncogenic potential of widespread deaminase-induced mutagenesis.

Though no perfect solution to spurious DNA editing currently exists, we note that sDA-BEs maintain robust on-target editing rates more favorably than the reduced-activity CBEs such as SECURE variants, YE variants, or AALN, and therefore may be an option for CBE applications that require high specificity but cannot afford an activity cost. As evidenced by comparison against a BE4Max titration experiment, we show that sDA-BEs do not appear to limit spurious editing via a simple concentration-limiting mechanism. Instead, we reason that nCas9-mediated accumulation of the sDA2 piece at the on-target site may create a “primed” state in which the nCas9-tethered sDA2 waits for an interaction with its cognate sDA1 piece from solution, after which a reformed enzymatic machinery can proceed with deamination as normal. Since no such nCas9-mediated accumulation exists at spurious off-target sites, such editing events must therefore rely on a lucky collision of all three “reactants” (both sDA pieces and an ssDNA substrate). In this model, spurious DNA editing is reduced with sDA-BEs compared to their intact parent enzymes because of a reduced likelihood, on top of whatever concentration-limiting effects. In the future, creation of a dual-targeted sDA-BE that uses two adjacently-targeted DNA binding domains may further improve both on-target editing efficiency and specificity, as such configurations should improve an accumulative mechanism that may bias sDA-BE editing away from spurious target sites.

### Programmable DNA Binding Domains

The split deaminase base editors described herein, e.g., fusion proteins of the split deaminases, can include programmable DNA binding domains such as engineered C2H2 zinc-fingers, transcription activator effector-like effectors (TALEs), and Clustered Regularly Interspaced Short Palindromic Repeats (CRISPR) Cas RNA-guided nucleases (RGNs) and their variants, including ssDNA nickases (nCas9) or their analogs and catalytically inactive dead Cas9 (dCas9) and its analogs, and any engineered protospacer-adjacent motif (PAM) variants. A programmable DNA binding domain is one that can be engineered to bind to a selected target sequence.

CRISPR-Cas Nucleases

Although herein we refer to nCas9, in general any Cas9-like nickase could be used based on any ortholog of the Cpf1 protein (including the related Cpf1 enzyme class), unless specifically indicated, including, e.g., those shown in Tables 1A and 1B.

TABLE 1A
List of Exemplary Cas9 Orthologs

| | | Nickase |
|---|---|---|
| | | Mutations/ |
| | UniProt Accession | Catalytic |
| Ortholog | Number | residues |
| S. pyogenes Cas9 (SpCas9) | Q99ZW2 | D10A, E762A, |
| | | H840A, N854A, |
| | | N863A, D986A¹⁷ |
| S. aureus Cas9 (SaCas9) | J7RUA5 | D10A and |
| | | N580¹⁸ |
| S. thermophilus Cas9 | G3ECR1 | D31A and |
| (St1Cas9) | | N891A¹⁹ |
| S. pasteuriamus Cas9 | F5X275 | D10, H599* |
| (SpaCas9) | | |
| C. jejuni Cas9 (CjCas9) | Q0P897 | D8A, H559A²⁰ |
| E novicida Cas9 (FnCas9) | A0Q5Y3 | D11, X995²¹ |
| P. lavamentivorans Cas9 | A7HP89 | D8, H601* |
| (PlCas9) | | |
| C. lari Cas9 (ClCas9) | G1UFN3 | D7, H567* |
| F. novicida Cpfl (FnCpf1) | A0Q7Q2 | D917, E1006, |
| | | D1255²¹ |
| M. bovoculi Cpfl (MbCpf1) | Sequence given at end | N/A** |
| A. sp. BV3L6 (AsCpf1) | U2UMQ6 | D908, 993E, |
| | | Q1226, D1263²³ |
| L. bacterium N2006 (LbCpf1) | A0A182DWE3 | D832A²⁴ |

*predicted based on UniRule annotation on the UniProt database.
**May be determinable based on sequence alignment with other Cpf1 orthologs

These orthologs, and mutants and variants thereof as known in the art, can be used in any of the split deaminases described herein. See, e.g., WO 2017/040348 (which describes variants of SaCas9 and SpCas 9 with increased specificity) and WO 2016/141224 (which describes variants of SaCas9 and SpCas 9 with altered PAM specificity).

TABLE 1B
List of Exemplary High Fidelity and/or PAM-relaxed RGN
Orthologs

| Published | | |
|---|---|---|
| HF/PAM-RGN | | |
| variants | PMID | Mutations* |
| S. pyogenes Cas9 | 26628643 | K810A/K1003A/R1060A(1.0); |
| (SpCas9) | | K848A/K1003A/R1060A(l.l) |
| eSpCas9 | | |
| S. pyogenes Cas9 | 29431739 | M495V/Y515N/K526E/R661Q; |
| (SpCas9) | | (M495V/Y515N/K526E/R661S; |
| evoCas9 | | M495V/Y515N/K526E/R66 IL) |
| S. pyogenes Cas9 | 26735016 | N497A/R661A/Q695A/Q926A |
| (SpCas9) HF1 | | |
| S. pyogenes Cas9 | 30082871 | R691A |
| (SpCas9) HiFi | | |
| Cas9 | | |
| S. pyogenes Cas9 | 28931002 | N692A, M694A, Q695A, H698A |
| (SpCas9) | | |
| HypaCas9 | | |
| S. pyogenes Cas9 | 30082838 | F539S, M763I, K890N |
| (SpCas9) Sniper- | | |
| Cas9 | | |
| S. pyogenes Cas9 | 29512652 | A262T, R324L, S409I, E480K, E543D, M694I, |
| (SpCas9) xCas9 | | E1219V |
| S. pyogenes Cas9 | 30166441 | R1335V, L1111R, D1135V, G1218R, |
| (SpCas9) | | E1219F, A1322R, T1337R |
| SpCas9-NG | | |
| S. pyogenes Cas9 | 26098369 | D1135V, R1335Q, T1337R; |
| (SpCas9) | | D1135V/G1218R/R1335E/T1337R |
| VQR/VRER | | |
| S. aureus Cas9 | 26524662 | E782K/N968K/R1015H |
| (SaCas9)-KKH | | |
| enAsCas12a | USSN 15/960,271 | One or more of: E174R, S170R, S542R, K548R, |
| | | K548V, N551R, N552R, K607R, K607H, e.g., |
| | | E174R/S542R/K548R, E174R/S542R/K607R, |
| | | E174R/S542R/K548V/N552R, |
| | | S170R/S542R/K548R, S170R/E174R, E174R/S542R, |
| | | S170R/S542R, E174R/S542R/K548R/N551R, |
| | | E174R/S542R/K607H, S170R/S542R/K607R, or |
| | | S170R/S542R/K548V/N552R |
| enAsCas12a-HF | USSN 15/960,271 | One or more of: E174R, S542R, K548R, e.g., |
| | | E174R/S542R/K548R, E174R/S542R/K607R, |
| | | E174R/S542R/K548V/N552R, |
| | | S170R/S542R/K548R, S170R/E174R, E174R/S542R, |
| | | S170R/S542R, E174R/S542R/K548R/N551R, |
| | | E174R/S542R/K607H, S170R/S542R/K607R, or |
| | | S170R/S542R/K548V/N552R, with the addition of |
| | | one or more of: N282A, T315A, N515A and K949A |
| enLbCas12a(HF) | USSN 15/960,271 | One or more of T152R, T152K, D156R, D156K, |
| | | Q529K, G532R, G532K, G532Q, K538R, K538V, |
| | | D541R, Y542R, M592A, K595R, K595H, K595S or |
| | | K595Q, e.g., D156R/G532R/K538R, |
| | | D156R/G532R/K595R, |
| | | D156R/G532R/K538V/Y542R, |
| | | T152R/G532R/K538R, T152R/D156R, |
| | | D156R/G532R, T152R/G532R, |
| | | D156R/G532R/K53 8R/D541R, |
| | | D156R/G532R/K595H, T152R/G532R/K595R, |
| | | T152R/G532R/K538V/Y542R, optionally with the |
| | | addition of one or more of: N260A, N256A, K514A, |
| | | D505A, K881A, S286A, K272A, K897A |
| enFnCas12a(HF) | USSN 15/960,271 | One or more of T177A, K180R, K180K, E184R, |
| | | E184K, T604K, N607R, N607K, N607Q, K613R, |
| | | K613V, D616R, N617R, M668A, K671R, K671H, |
| | | K671S, orK671Q, e.g., E184R/N607R/K613R, |
| | | E184R/N607R/K671R, |
| | | E184R/N607R/K613 V/N617R, |
| | | K180R/N607R/K613R, K180R/E184R, |
| | | E184R/N607R, K180R/N607R, |
| | | E184R/N607R/K613R/D616R, |
| | | E184R/N607R/K671H, K180R/N607R/K671R, |
| | | K180R/N607R/K613V/N617R, optionally with the |
| | | addition of one or more of: N305A, N301 A, K589A, |
| | | N580A, K962A, S334A, K320A, K978A |
| S. pyogenes Cas9 | 32217751 | D1135L, S1136W, G1218K, E1219Q, R1335Q, |
| (SpGas9) SpG | | T1337R |
| S. pyogenes Cas9 | 32217751 | A61R, L1111R, D1135L, S1136W, G1218K, |
| (SpGas9) SpRY | | E1219Q, N1317R, A1322R, R1333P, R1335Q, |
| | | T1337R |

*predicted based on UniRule annotation on the UniProt database.

The Cas9 nuclease from S. pyogenes (hereafter simply Cas9) can be guided via simple base pair complementarity between 17-20 nucleotides of an engineered guide RNA (gRNA), e.g., a single guide RNA or crRNA/tracrRNA pair, and the complementary strand of a target genomic DNA sequence of interest that lies next to a protospacer adjacent motif (PAM), e.g., a PAM matching the sequence NGG or NAG (Shen et al., Cell Res (2013); Dicarlo et al., Nucleic Acids Res (2013); Jiang et al., Nat Biotechnol 31, 233-239 (2013); Jinek et al., Elife 2, e00471 (2013); Hwang et al., Nat Biotechnol 31, 227-229 (2013); Cong et al., Science 339, 819-823 (2013); Mali et al., Science 339, 823-826 (2013c); Cho et al., Nat Biotechnol 31, 230-232 (2013); Jinek et al., Science 337, 816-821 (2012)). The engineered CRISPR from Prevotella and Francisella 1 (Cpf1) nuclease can also be used, e.g., as described in Zetsche et al., Cell 163, 759-771 (2015); Schunder et al., Int J Med Microbiol 303, 51-60 (2013); Makarova et al., Nat Rev Microbiol 13, 722-736 (2015); Fagerlund et al., Genome Biol 16, 251 (2015). Unlike SpCas9, Cpf1 requires only a single 42-nt crRNA, which has 23 nt at its 3′ end that are complementary to the protospacer of the target DNA sequence (Zetsche et al., 2015). Furthermore, whereas SpCas9 recognizes an NGG PAM sequence that is 3′ of the protospacer, AsCpf1 and LbCpl recognize TTTN PAMs that are found 5′ of the protospacer (Id.).

The wild-type sequence of spCas9 (SEQ ID NO:50) is as follows:

MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

Wild-type spCas9 has 2 endonuclease domains. The discontinuous RuvC-like domain (approximately residues 1-62, 718-765 and 925-1102) recognizes and cleaves the target DNA noncomplementary to crRNA while the HNH nuclease domain (residues 810-872) cleaves the target DNA complementary to crRNA. See Jinek et al., “A Programmable Dual-RNA-Guided DNA Endonuclease in Adaptive Bacterial Immunity,” Science 337:816-21 (2012) and Nishimasu et al., “Crystal Structure of Cas9 in Complex with Guide RNA and Target DNA,” Cell 156:935-49 (2014).

Wild-type spCas9 has a bilobed architecture with a recognition lobe (REC, residues 60-718) and a discontinuous nuclease lobe (NUC, residues 1-59 and 719-1368). See Nishimasu et al., “Crystal Structure of Cas9 in Complex with Guide RNA and Target DNA,” Cell 156:935-49 (2014); Jiang et al., “A Cas9-Guide RNA Complex Preorganized for Target DNA Recognition,” Science 348:1477-81 (2015); and Jinek et al., “Structures of Cas9 endonucleases reveal RNA-mediated conformational activation,” Science 343:154997 (2014). The crRNA-target DNA lies in a channel between the 2 lobes (See Nishimasu et al., “Crystal Structure of Cas9 in Complex with Guide RNA and Target DNA,” Cell 156:935-49 (2014); Jiang et al., “A Cas9-Guide RNA Complex Preorganized for Target DNA Recognition,” Science 348:1477-81 (2015); and Jiang et al, “Structures of a CRISPR_Cas9 R-loop Complex Primed for DNA Cleavage,” Science 351:867-71 (2016)). Binding of sgRNA induces large conformational changes further enhanced by target DNA binding (see Jiang et al., “STRUCTURAL BIOLOGY. A Cas9-guide RNA Complex Preorganized for Target DNA Recognition,” Science 348:1477-81 (2015); and Jiang et al, “Structures of a CRISPR_Cas9 R-loop Complex Primed for DNA Cleavage,” Science 351:867-71 (2016)). REC recognizes and binds differing regions of an artifical sgRNA in a sequence-independent manner. Deletions of parts of this lobe abolish nuclease activity (See Nishimasu et al., “Crystal Structure of Cas9 in Complex with Guide RNA and Target DNA,” Cell 156:935-49 (2014)).

The PAM-interacting domain of wild-type spCas9 (PI domain, approximately residues 1099-1368) recognizes the PAM motif, swapping the PI domain of this enzyme with that from S. thermophilus St3Cas9 (AC Q03JI6) prevents cleavage of DNA with the endogenous PAM site (5′-NGG-3′) but confers the ability to cleave DNA with the PAM site specific for St3 CRISPRs. See Nishimasu et al., “Crystal Structure of Cas9 in Complex with Guide RNA and Target DNA,” Cell 156:935-49 (2014).

In some embodiments, the split deaminase base editors described herein, e.g., fusion proteins of the split deaminases utilizes a wild type or variant Cas9 protein from S. pyogenes or Staphylococcus aureus, or a wild type or variant Cpf1 protein from Acidaminococcus sp. BV3L6 or Lachnospiraceae bacterium ND2006 either as encoded in bacteria or codon-optimized for expression in mammalian cells and/or modified in its PAM recognition specificity and/or its genome-wide specificity. A number of variants have been described; see, e.g., WO 2016/141224, PCT/US2016/049147, Kleinstiver et al., Nat Biotechnol. 2016 August; 34(8):869-74; Tsai and Joung, Nat Rev Genet. 2016 May; 17(5):300-12; Kleinstiver et al., Nature. 2016 Jan. 28; 529(7587):490-5; Shmakov et al., Mol Cell. 2015 Nov. 5; 60(3):385-97; Kleinstiver et al., Nat Biotechnol. 2015 December; 33(12):1293-1298; Dahlman et al., Nat Biotechnol. 2015 November; 33(11):1159-61; Kleinstiver et al., Nature. 2015 Jul. 23; 523(7561):481-5; Wyvekens et al., Hum Gene Ther. 2015 Jul.; 26(7):425-31; Hwang et al., Methods Mol Biol. 2015; 1311:317-34; Osborn et al., Hum Gene Ther. 2015 Feb.; 26(2):114-26; Konermann et al., Nature. 2015 Jan. 29; 517(7536):583-8; Fu et al., Methods Enzymol. 2014; 546:21-45; and Tsai et al., Nat Biotechnol. 2014 June; 32(6):569-76, inter alia. Concerning rAPOBEC1 itself, a number of variants have been described, e.g. Chen et al, RNA. 2010 May; 16(5):1040-52; Chester et al, EMBO J. 2003 Aug. 1; 22(15):3971-82; Teng et al, J Lipid Res. 1999 April; 40(4):623-35.; Navaratnam et al, Cell. 1995 Apr. 21; 81(2):187-95; MacGinnitie et al, J Biol Chem. 1995 Jun. 16; 270(24):14768-75; Yamanaka et al, J Biol Chem. 1994 Aug. 26; 269(34):21725-34. The guide RNA is expressed or present in the cell together with the Cas9 or Cpf1. Either the guide RNA or the nuclease, or both, can be expressed transiently or stably in the cell or introduced as a purified protein or nucleic acid.

In some embodiments, the Cas9 also includes one of the following mutations, which reduce nuclease activity of the Cas9; e.g., for SpCas9, mutations at D10 (e.g., D10A) or H840 (e.g., H840A) (which creates a single-strand nickase).

In some embodiments, the SpCas9 variants also include mutations at one of each of the two sets of the following amino acid positions, which together destroy the nuclease activity of the Cas9: D10, E762, D839, H983, or D986 and H840 or N863, e.g., D10A/D10N and H840A/H840N/H840Y, to render the nuclease portion of the protein catalytically inactive; substitutions at these positions could be alanine (as they are in Nishimasu al., Cell 156, 935-949 (2014)), or other residues, e.g., glutamine, asparagine, tyrosine, serine, or aspartate, e.g., E762Q, H983N, H983Y, D986N, N863D, N863S, or N863H (see WO 2014/152432).

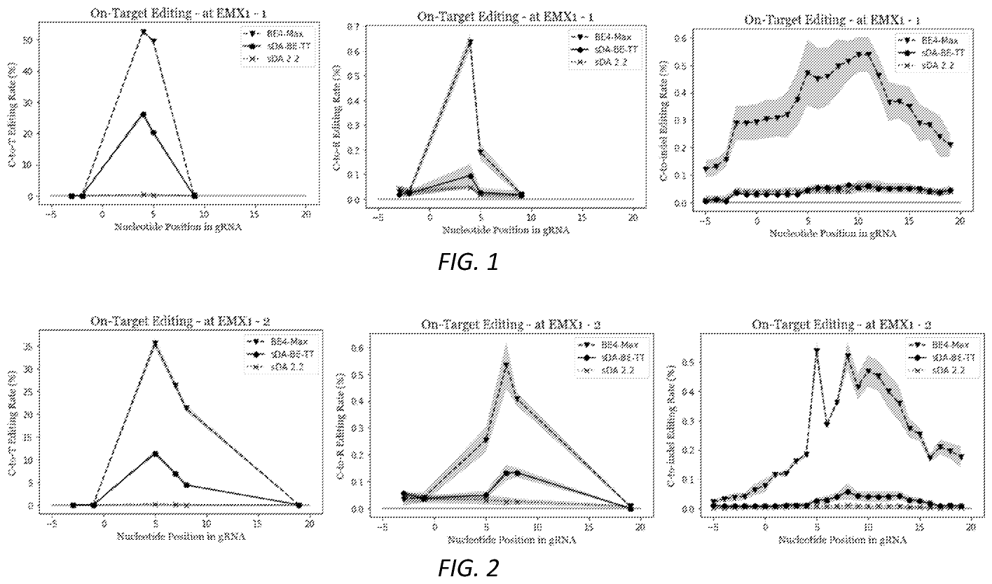

Cas9 molecules of a variety of species can be used in the methods and compositions described herein. While the S. pyogenes and S. thermophilus Cas9 molecules are the subject of much of the disclosure herein, Cas9 molecules of, derived from, or based on the Cas9 proteins of other species listed herein can be used as well. In other words, while the much of the description herein uses S. pyogenes and S. thermophilus Cas9 molecules, Cas9 molecules from the other species can replace them. Such species include those set forth in the following table, which was created based on supplementary FIG. 1 of Chylinski et al., 2013.

Alternative Cas9 proteins

| GenBank Acc No. | Bacterium |
|---|---|
| 303229466 | Veillonella atypica ACS-134-V-Col7a |
| 34762592 | Fusobacterium nucleatum subsp. vincentii |
| 374307738 | Filifactor alocis ATCC 35896 |
| 320528778 | Solobacterium moorei F0204 |
| 291520705 | Coprococcus catus GD-7 |
| 42525843 | Treponema denticola ATCC 35405 |
| 304438954 | Peptoniphilus duerdenii ATCC BAA-1640 |
| 224543312 | Catenibacterium mitsuokai DSM 15897 |
| 24379809 | Streptococcus mutans UA159 |
| 15675041 | Streptococcus pyogenes SF370 |
| 16801805 | Listeria innocua Clip11262 |
| 116628213 | Streptococcus thermophilus LMD-9 |
| 323463801 | Staphylococcus pseudintermedius ED99 |
| 352684361 | Acidaminococcus intestini RyC-MR95 |
| 302336020 | Olsenella uli DSM 7084 |
| 366983953 | Oenococcus kitaharae DSM 17330 |
| 310286728 | Bifidobacterium bifidum S17 |
| 258509199 | Lactobacillus rhamnosus GG |
| 300361537 | Lactobacillus gasseri JV-V03 |
| 169823755 | Finegoldia magna ATCC 29328 |
| 47458868 | Mycoplasma mobile 163K |
| 284931710 | Mycoplasma gallisepticum str. F |
| 363542550 | Mycoplasma ovipneumoniae SC01 |
| 384393286 | Mycoplasma canis PG 14 |
| 71894592 | Mycoplasma synoviae 53 |
| 238924075 | Eubacterium rectale ATCC 33656 |
| 116627542 | Streptococcus thermophilus LMD-9 |
| 315149830 | Enterococcus faecalis TX0012 |
| 315659848 | Staphylococcus lugdunensis M23590 |
| 160915782 | Eubacterium dolichum DSM 3991 |
| 336393381 | Lactobacillus coryniformis subsp. torquens |
| 310780384 | Ilyobacter polytropus DSM 2926 |
| 325677756 | Ruminococcus albus 8 |
| 187736489 | Akkermansia muciniphila ATCC BAA-835 |
| 117929158 | Acidothermus cellulolyticus 11B |
| 189440764 | Bifidobacterium longum DJO10A |
| 283456135 | Bifidobacterium dentium Bd1 |
| 38232678 | Corynebacterium diphtheriae NCTC 13129 |
| 187250660 | Elusimicrobium minutum Pei191 |
| 319957206 | Nitratifractor salsuginis DSM 16511 |
| 325972003 | Sphaerochaeta globus str. Buddy |
| 261414553 | Fibrobacter succinogenes subsp. succinogenes |
| 60683389 | Bacteroides fragilis NCTC 9343 |
| 256819408 | Capnocytophaga ochracea DSM 7271 |
| 90425961 | Rhodopseudomonas palustris BisB18 |
| 373501184 | Prevotella micans F0438 |
| 294674019 | Prevotella ruminicola 23 |
| 365959402 | Flavobacterium columnare ATCC 49512 |
| 312879015 | Aminomonas paucivorans DSM 12260 |
| 83591793 | Rhodospirillum rubrum ATCC 11170 |
| 294086111 | Candidatus Puniceispirillum marinum IMCC1322 |
| 121608211 | Verminephrobacter eiseniae EF01-2 |
| 344171927 | Ralstonia syzygii R24 |
| 159042956 | Dinoroseobacter shibae DFL 12 |
| 288957741 | Azospirillum sp- B510 |
| 92109262 | Nitrobacter hamburgensis X14 |
| 148255343 | Bradyrhizobium sp- BTAil |
| 34557790 | Wolinella succinogenes DSM 1740 |
| 218563121 | Campylobacter jejuni subsp. jejuni |
| 291276265 | Helicobacter mustelae 12198 |
| 229113166 | Bacillus cereus Rock1-15 |
| 222109285 | Acidovorax ebreus TPSY |
| 189485225 | uncultured Termite group 1 |
| 182624245 | Clostridium perfringens D str. |
| 220930482 | Clostridium cellulolyticum H10 |
| 154250555 | Parvibaculum lavamentivorans DS-1 |
| 257413184 | Roseburia intestinalis L1-82 |
| 218767588 | Neisseria meningitidis Z2491 |
| 15602992 | Pasteurella multocida subsp. multocida |
| 319941583 | Sutterella wadsworthensis 3 1 |
| 254447899 | gamma proteobacterium HTCC5015 |
| 54296138 | Legionella pneumophila str. Paris |
| 331001027 | Parasutterella excrementihominis YIT 11859 |
| 34557932 | Wolinella succinogenes DSM 1740 |
| 118497352 | Francisella novicida U112 |

The split deaminase base editors described herein, e.g., fusion proteins of the split deaminases, can include the use of any of those Cas9 proteins, and their corresponding guide RNAs or other guide RNAs that are compatible. The Cas9 from Streptococcus thermophilus LMD-9 CRISPR1 system has been shown to function in human cells in Cong et al (Science 339, 819 (2013)). Additionally, Jinek et al. showed in vitro that Cas9 orthologs from S. thermophilus and L. innocua, (but not from N. meningitidis or C. jejuni, which likely use a different guide RNA), can be guided by a dual S. pyogenes gRNA to cleave target plasmid DNA, albeit with slightly decreased efficiency.

In some embodiments, the Cas9 is fused to one or more Uracil glycosylase inhibitor (UGI) protein sequences; an exemplary UGI sequence is as follows:

(SEQ ID NO:47; Uniprot: P14739)
TNLSDIIEKETGKQLVIQESILMLPEEVEEVIGNKPESDILVHTAYDEST
DENVMLLTSDAPEYKPWALVIQDSNGENKIKML.

Typically, the UGIs are at the C-terminus of a BE fusion protein, but could conceivably be at the N-terminus, or between the DNA binding domain and the sDA domain. Linkers as known in the art can be used to separate domains.

TAL Effector Repeat Arrays

Transcription activator like effectors (TALEs) of plant pathogenic bacteria in the genus Xanthomonas play important roles in disease, or trigger defense, by binding host DNA and activating effector-specific host genes. Specificity depends on an effector-variable number of imperfect, typically ˜33-35 amino acid repeats. Polymorphisms are present primarily at repeat positions 12 and 13, which are referred to herein as the repeat variable-diresidue (RVD). The RVDs of TAL effectors correspond to the nucleotides in their target sites in a direct, linear fashion, one RVD to one nucleotide, with some degeneracy and no apparent context dependence. In some embodiments, the polymorphic region that grants nucleotide specificity may be expressed as a triresidue or triplet.

Each DNA binding repeat can include a RVD that determines recognition of a base pair in the target DNA sequence, wherein each DNA binding repeat is responsible for recognizing one base pair in the target DNA sequence. In some embodiments, the RVD can comprise one or more of: HA for recognizing C; ND for recognizing C; HI for recognizing C; HN for recognizing G; NA for recognizing G; SN for recognizing G or A; YG for recognizing T; and NK for recognizing G, and one or more of: HD for recognizing C; NG for recognizing T; NI for recognizing A; NN for recognizing G or A; NS for recognizing A or C or G or T; N* for recognizing C or T, wherein * represents a gap in the second position of the RVD; HG for recognizing T; H* for recognizing T, wherein * represents a gap in the second position of the RVD; and IG for recognizing T.

TALE proteins may be useful in research and biotechnology as targeted chimeric nucleases that can facilitate homologous recombination in genome engineering (e.g., to add or enhance traits useful for biofuels or biorenewables in plants). These proteins also may be useful as, for example, transcription factors, and especially for therapeutic applications requiring a very high level of specificity such as therapeutics against pathogens (e.g., viruses) as non-limiting examples.

Methods for generating engineered TALE arrays are known in the art, see, e.g., the fast ligation-based automatable solid-phase high-throughput (FLASH) system described in U.S. Ser. No. 61/610,212, and Reyon et al., Nature Biotechnology 30, 460-465 (2012); as well as the methods described in Bogdanove & Voytas, Science 333, 1843-1846 (2011); Bogdanove et al., Curr Opin Plant Biol 13, 394-401 (2010); Scholze & Boch, J. Curr Opin Microbiol (2011); Boch et al., Science 326, 1509-1512 (2009); Moscou & Bogdanove, Science 326, 1501 (2009); Miller et al., Nat Biotechnol 29, 143-148 (2011); Morbitzer et al., T. Proc Natl Acad Sci USA 107, 21617-21622 (2010); Morbitzer et al., Nucleic Acids Res 39, 5790-5799 (2011); Zhang et al., Nat Biotechnol 29, 149-153 (2011); Geissler et al., PLoS ONE 6, e19509 (2011); Weber et al., PLoS ONE 6, e19722 (2011); Christian et al., Genetics 186, 757-761 (2010); Li et al., Nucleic Acids Res 39, 359-372 (2011); Mahfouz et al., Proc Natl Acad Sci USA 108, 2623-2628 (2011); Mussolino et al., Nucleic Acids Res (2011); Li et al., Nucleic Acids Res 39, 6315-6325 (2011); Cermak et al., Nucleic Acids Res 39, e82 (2011); Wood et al., Science 333, 307 (2011); Hockemeye et al. Nat Biotechnol 29, 731-734 (2011); Tesson et al., Nat Biotechnol 29, 695-696 (2011); Sander et al., Nat Biotechnol 29, 697-698 (2011); Huang et al., Nat Biotechnol 29, 699-700 (2011); and Zhang et al., Nat Biotechnol 29, 149-153 (2011); all of which are incorporated herein by reference in their entirety.

Also suitable for use in the present methods are MegaTALs, which are a fusion of a meganuclease with a TAL effector; see, e.g., Boissel et al., Nucl. Acids Res. 42(4):2591-2601 (2014); Boissel and Scharenberg, Methods Mol Biol. 2015; 1239:171-96.

Zinc Fingers

Zinc finger (ZF) proteins are DNA-binding proteins that contain one or more zinc fingers, independently folded zinc-containing mini-domains, the structure of which is well known in the art and defined in, for example, Miller et al., 1985, EMBO J., 4:1609; Berg, 1988, Proc. Natl. Acad. Sci. USA, 85:99; Lee et al., 1989, Science. 245:635; and Klug, 1993, Gene, 135:83. Crystal structures of the zinc finger protein Zif268 and its variants bound to DNA show a semi-conserved pattern of interactions, in which typically three amino acids from the alpha-helix of the zinc finger contact three adjacent base pairs or a “subsite” in the DNA (Pavletich et al., 1991, Science, 252:809; Elrod-Erickson et al., 1998, Structure, 6:451). Thus, the crystal structure of Zif268 suggested that zinc finger DNA-binding domains might function in a modular manner with a one-to-one interaction between a zinc finger and a three-base-pair “subsite” in the DNA sequence. In naturally occurring zinc finger transcription factors, multiple zinc fingers are typically linked together in a tandem array to achieve sequence-specific recognition of a contiguous DNA sequence (Klug, 1993, Gene 135:83).

Multiple studies have shown that it is possible to artificially engineer the DNA binding characteristics of individual zinc fingers by randomizing the amino acids at the alpha-helical positions involved in DNA binding and using selection methodologies such as phage display to identify desired variants capable of binding to DNA target sites of interest (Rebar et al., 1994, Science, 263:671; Choo et al., 1994 Proc. Natl. Acad. Sci. USA, 91:11163; Jamieson et al., 1994, Biochemistry 33:5689; Wu et al., 1995 Proc. Natl. Acad. Sci. USA, 92: 344). Such recombinant zinc finger proteins can be fused to functional domains, such as transcriptional activators, transcriptional repressors, methylation domains, and nucleases to regulate gene expression, alter DNA methylation, and introduce targeted alterations into genomes of model organisms, plants, and human cells (Carroll, 2008, Gene Ther., 15:1463-68; Cathomen, 2008, Mol. Ther., 16:1200-07; Wu et al., 2007, Cell. Mol. Life Sci., 64:2933-44).

One existing method for engineering zinc finger arrays, known as “modular assembly,” advocates the simple joining together of pre-selected zinc finger modules into arrays (Segal et al., 2003, Biochemistry, 42:2137-48; Beerli et al., 2002, Nat. Biotechnol., 20:135-141; Mandell et al., 2006, Nucleic Acids Res., 34:W516-523; Carroll et al., 2006, Nat. Protoc. 1:1329-41; Liu et al., 2002, J. Biol. Chem., 277:3850-56; Bae et al., 2003, Nat. Biotechnol., 21:275-280; Wright et al., 2006, Nat. Protoc., 1:1637-52). Although straightforward enough to be practiced by any researcher, recent reports have demonstrated a high failure rate for this method, particularly in the context of zinc finger nucleases (Ramirez et al., 2008, Nat. Methods, 5:374-375; Kim et al., 2009, Genome Res. 19:1279-88), a limitation that typically necessitates the construction and cell-based testing of very large numbers of zinc finger proteins for any given target gene (Kim et al., 2009, Genome Res. 19:1279-88).

Combinatorial selection-based methods that identify zinc finger arrays from randomized libraries have been shown to have higher success rates than modular assembly (Maeder et al., 2008, Mol. Cell, 31:294-301; Joung et al., 2010, Nat. Methods, 7:91-92; Isalan et al., 2001, Nat. Biotechnol., 19:656-660). In preferred embodiments, the zinc finger arrays are described in, or are generated as described in, WO 2011/017293 and WO 2004/099366. Additional suitable zinc finger DBDs are described in U.S. Pat. Nos. 6,511,808, 6,013,453, 6,007,988, and 6,503,717 and U.S. patent application 2002/0160940.

### Deaminases

In some embodiments, the split deaminase base editor described herein, e.g., fusion proteins of the split deaminases, comprises a deaminase that modifies cytosine DNA bases, e.g., a cytosine deaminase from the apolipoprotein B mRNA-editing enzyme, catalytic polypeptide-like (APOBEC) family of deaminases, including APOBEC1, APOBEC2, APOBEC3A, APOBEC3B, APOBEC3C, APOBEC3D/E, APOBEC3F, APOBEC3G, APOBEC3H, APOBEC4 (see, e.g., Yang et al., J Genet Genomics. 2017 Sep. 20; 44(9):423-437); activation-induced cytosine deaminase (AID), e.g., activation induced cytosine deaminase (AICDA), cytosine deaminase 1 (CDA1), and CDA2, and cytosine deaminase acting on tRNA (CDAT). The following Table 2 provides exemplary sequences; other sequences can also be used.

TABLE 2
Exemplary Deaminase Sequences.

| | GenBank Accession Nos. | |
|---|---|---|
| Deaminase | Nucleic Acid | Amino Acid |
| hAID/AICDA | NM_020661.3 isoform 1 | NP_065712.1 variant 1 |
| | NM_020661.3 isoform 2 | NP_065712.1 variant 2 |
| APOBEC1 | NM_001644.4 isoform a | NP_001635.2 variant 1 |
| | NM_005889.3 isoform b | NP_005880.2 variant 3 |
| APOBEC2 | NM_006789.3 | NP_006780.1 |
| APOBEC3A | NM_145699.3 isoform a | NP_663 745.1 variant 1 |
| | NM_001270406.1 isoform b | NP_001257335.1 variant 2 |
| APOBEC3B | NM_004900.4 isoform a | NP_004891.4 variant 1 |
| | NM_001270411.1 isoform b | NP_001257340.1 variant 2 |
| APOBEC3C | NM_014508.2 | NP_055323.2 |
| APOBEC3D/E | NM_152426.3 | NP_689639.2 |
| APOBEC3F | NM_145298.5 isoform a | NP_660341.2 variant 1 |
| | NM_001006666.1 isoform b | NP_001006667.1 variant 2 |
| APOBEC3G | NM_021822.3 (isoform a) | NP_068594.1 (variant 1) |
| APOBEC3H | NM_001166003.2 | NP_001159475.2 (variant |
| | | SV-200) |
| APOBEC4 | NM_203454.2 | NP_982279.1 |
| CDA1* | NM_27515.4 | NP_179547.1 |
| yCD (FCY1)* | NM_001184159.1 | NP_0153 87.1 |

*from Saccharomyces cerevisiae S288C

Exemplary split deaminase regions are shown in Table 3. Each split region listed in Table 3 represents a region of the enzyme either known to be a linker region devoid of secondary structure and positioned away from enzymatically important functions or predicted to be linker based on alignment with hAPOBEC3G where structural information is lacking (* indicates which proteins lack sufficient structural information). Unstructured recognition loops were not included due to their importance in determining substrate binding and specificity. All protein sequences acquired from uniprot.org. All positional information refers to positions within the full-length protein sequences as described below. Candidate split regions described only indicate our best attempt at a priori prediction of which splits will be functional.

TABLE 3
Exemplary Split Deaminase Regions

| | Split | Split | Split | Split | Split | Split |
|---|---|---|---|---|---|---|
| Deaminase | Region 1 | Region 2 | Region 3 | Region 4 | Region 5 | Region 6 |
| hAID | N51-H56 | D69-C75 | S85-P86 | P102-N103 | M129-T140 | E153-E163 |
| rAPOBEC1* | H48-H61 | Y75-R81 | S91-P92 | P108-H109 | M144-T145 | N158-W167 |
| mAPOBEC3* | N66-I70 | V87-E93 | S103-P104 | H120-N121 | M156-D157 | D170-K180 |
| hAPOBEC3A | N57-H70 | Q83-I89 | S99-P100 | T118-H119 | M153-T154 | D167-G178 |
| hAPOBEC3C | N57-H66 | I79-K85 | S95-P96 | S112-N113 | M148-D149 | Y162-K172 |
| hAPOBEC3G | N244-H257 | K270-D276 | S286-P287 | K303-H304 | M338-T339 | D352-D362 |
| hAPOBEC3H* | N49-H54 | K67-C73 | S83-P84 | D100-H101 | M136-G137 | D150-Y160 |
| hAPOBEC3F | N240-H249 | I262-N268 | S278-P279 | S295-N296 | M331-G332 | Y345-K355 |

The split deaminase regions can include mutations that may enhance base editing, e.g., when made to the nCas9-UGI portion, e.g., mutations corresponding to W90, R126, or R132 of rAPOBEC1 (rAPO1), e.g., corresponding to W90Y, R126E, R132E, of rAPOBEC1 (rAPO1) (see, e. g., Kim et al. “Increasing the Genome-Targeting Scope and Precision of Base Editing with Engineered Cas9-Cytosine Deaminase Fusions.” Nature Biotechnology 35(4):371-376 (2017); U.S. Patent Application Publication No. 2020/0172895). Alternatively or in addition, the split deaminase regions can include mutations at positions corresponding to one or more of N57, Y130, or K60 of SEQ ID NO:49, e.g., mutations corresponding to N57G, N57A, N57Q, Y130F, K60D of hAPOBEC3A (hA3A) (see, e.g. U.S. Patent Application Publication No. 2020/0172895).

### Variants

In some embodiments, the split deaminase base editors described herein and/or the components of the split deaminase base editors described herein, e.g., fusion proteins of the split deaminase base editors, are at least 80%, e.g., at least 85%, 90%, 95%, 97%, or 99% identical to the amino acid sequence of a exemplary sequence (e.g., as provided herein), e.g., have differences at up to 1%, 2%, 5%, 10%, 15%, or 20% of the residues of the exemplary sequence replaced, e.g., with conservative mutations, e.g., including or in addition to the mutations described herein. In preferred embodiments, the variant retains desired activity of the parent, e.g., nickase activity, and/or the ability to interact with a guide RNA and/or target DNA, optionally with improved specificity or altered substrate specificity.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences. Percent identity between two polypeptides or nucleic acid sequences is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins or nucleic acids, the length of comparison can be any length, up to and including full length (e.g., 5%, 10%, 20%, 30%, 40%, 50%, 60%, 70%, 80%, 90%, 95%, or 100%). For purposes of the present compositions and methods, at least 80% of the full length of the sequence is aligned.

For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Also provided herein are isolated nucleic acids encoding the split deaminase base editors described herein, e.g., fusion proteins of the split deaminases and, vectors comprising the isolated nucleic acids, optionally operably linked to one or more regulatory domains for expressing the variant proteins, and host cells, e.g., mammalian host cells, comprising the nucleic acids, and optionally expressing the variant proteins. In some embodiments, the host cells are stem cells, e.g., hematopoietic stem cells.

In some embodiments, the split deaminase base editors described herein comprise fusion proteins, e.g., a fusion protein comprising a DNA binding domain and a BE domain. In some embodiments, the fusion proteins include a linker between the DNA binding domain (e.g., ZFN, TALE, or nCas9) and the BE domains. Linkers that can be used in these fusion proteins (or between fusion proteins in a concatenated structure) can include any sequence that does not interfere with the function of the fusion proteins. In preferred embodiments, the linkers are short, e.g., 2-20 amino acids, and are typically flexible (i.e., comprising amino acids with a high degree of freedom such as glycine, alanine, and serine). In some embodiments, the linker comprises one or more units consisting of GGGS (SEQ ID NO:75) or GGGGS (SEQ ID NO:76), e.g., two, three, four, or more repeats of the GGGS (SEQ ID NO:75) or GGGGS (SEQ ID NO:76) unit. Other linker sequences can also be used.

In some embodiments, split deaminase base editors described herein, e.g., fusion proteins of the split deaminases, include a cell-penetrating peptide sequence that facilitates delivery to the intracellular space, e.g., HIV-derived TAT peptide, penetratins, transportans, or hCT derived cell-penetrating peptides, see, e.g., Caron et al., (2001) Mol Ther. 3(3):310-8; Langel, Cell-Penetrating Peptides: Processes and Applications (CRC Press, Boca Raton Fla. 2002); El-Andaloussi et al., (2005) Curr Pharm Des. 11(28):3597-611; and Deshayes et al., (2005) Cell Mol Life Sci. 62(16):1839-49.

Cell penetrating peptides (CPPs) are short peptides that facilitate the movement of a wide range of biomolecules across the cell membrane into the cytoplasm or other organelles, e.g. the mitochondria and the nucleus. Examples of molecules that can be delivered by CPPs include therapeutic drugs, plasmid DNA, oligonucleotides, siRNA, peptide-nucleic acid (PNA), proteins, peptides, nanoparticles, and liposomes. CPPs are generally 30 amino acids or less, are derived from naturally or non-naturally occurring protein or chimeric sequences, and contain either a high relative abundance of positively charged amino acids, e.g. lysine or arginine, or an alternating pattern of polar and non-polar amino acids. CPPs that are commonly used in the art include Tat (Frankel et al., (1988) Cell. 55:1189-1193, Vives et al., (1997) J. Biol. Chem. 272:16010-16017), penetratin (Derossi et al., (1994) J. Biol. Chem. 269:10444-10450), polyarginine peptide sequences (Wender et al., (2000) Proc. Natl. Acad. Sci. USA 97:13003-13008, Futaki et al., (2001) J. Biol. Chem. 276:5836-5840), and transportan (Pooga et al., (1998) Nat. Biotechnol. 16:857-861).

CPPs can be linked with their cargo through covalent or non-covalent strategies. Methods for covalently joining a CPP and its cargo are known in the art, e.g. chemical cross-linking (Stetsenko et al., (2000) J. Org. Chem. 65:4900-4909, Gait et al. (2003) Cell. Mol. Life. Sci. 60:844-853) or cloning a fusion protein (Nagahara et al., (1998) Nat. Med. 4:1449-1453). Non-covalent coupling between the cargo and short amphipathic CPPs comprising polar and non-polar domains is established through electrostatic and hydrophobic interactions.

CPPs have been utilized in the art to deliver potentially therapeutic biomolecules into cells. Examples include cyclosporine linked to polyarginine for immunosuppression (Rothbard et al., (2000) Nature Medicine 6(11):1253-1257), siRNA against cyclin B1 linked to a CPP called MPG for inhibiting tumorigenesis (Crombez et al., (2007) Biochem Soc. Trans. 35:44-46), tumor suppressor p53 peptides linked to CPPs to reduce cancer cell growth (Takenobu et al., (2002) Mol. Cancer Ther. 1(12):1043-1049, Snyder et al., (2004) PLoS Biol. 2:E36), and dominant negative forms of Ras or phosphoinositol 3 kinase (PI3K) fused to Tat to treat asthma (Myou et al., (2003) J. Immunol. 171:4399-4405).

CPPs have been utilized in the art to transport contrast agents into cells for imaging and biosensing applications. For example, green fluorescent protein (GFP) attached to Tat has been used to label cancer cells (Shokolenko et al., (2005) DNA Repair 4(4):511-518). Tat conjugated to quantum dots have been used to successfully cross the blood-brain barrier for visualization of the rat brain (Santra et al., (2005) Chem. Commun. 3144-3146). CPPs have also been combined with magnetic resonance imaging techniques for cell imaging (Liu et al., (2006) Biochem. and Biophys. Res. Comm. 347(1):133-140). See also Ramsey and Flynn, Pharmacol Ther. 2015 Jul. 22. pii: S0163-7258(15)00141-2.

Alternatively or in addition, the split deaminase base editors described herein, e.g., fusion proteins of the split deaminases, can include a nuclear localization sequence, e.g., SV40 large T antigen NLS (PKKKRRV (SEQ ID NO:48)) and nucleoplasmin NLS (KRPAATKKAGQAKKKK (SEQ ID NO:49)). Other NLSs are known in the art; see, e.g., Cokol et al., EMBO Rep. 2000 Nov. 15; 1(5): 411-415; Freitas and Cunha, Curr Genomics. 2009 Dec.; 10(8): 550-557.

In some embodiments, the split deaminase base editors described herein, e.g., fusion proteins of the split deaminases, include a moiety that has a high affinity for a ligand, for example GST, FLAG or hexahistidine sequences. Such affinity tags can facilitate the purification of recombinant split deaminases, e.g., split deaminase fusion protein(s).

The split deaminase base editors described herein, e.g., fusion proteins of the split deaminases, can be used for altering the genome of a cell. The methods generally include expressing or contacting the split deaminase base editors, e.g., split deaminase fusion protein(s), in the cells; in versions using one or two Cas9s, the methods include using a guide RNA having a region complementary to a selected portion of the genome of the cell. Methods for selectively altering the genome of a cell are known in the art, see, e.g., U.S. Pat. No. 8,993,233; US 20140186958; U.S. Pat. No. 9,023,649; WO/2014/099744; WO 2014/089290; WO2014/144592; WO144288; WO2014/204578; WO2014/152432; WO2115/099850; U.S. Pat. No. 8,697,359; US20160024529; US20160024524; US20160024523; US20160024510; US20160017366; US20160017301; US20150376652; US20150356239; US20150315576; US20150291965; US20150252358; US20150247150; US20150232883; US20150232882; US20150203872; US20150191744; US20150184139; US20150176064; US20150167000; US20150166969; US20150159175; US20150159174; US20150093473; US20150079681; US20150067922; US20150056629; US20150044772; US20150024500; US20150024499; US20150020223; US20140356867; US20140295557; US20140273235; US20140273226; US20140273037; US20140189896; US20140113376; US20140093941; US20130330778; US20130288251; US20120088676; US20110300538; US20110236530; US20110217739; US20110002889; US20100076057; US20110189776; US20110223638; US20130130248; US20150050699; US20150071899; US20150050699; US20150045546; US20150031134; US20150024500; US20140377868; US20140357530; US20140349400; US20140335620; US20140335063; US20140315985; US20140310830; US20140310828; US20140309487; US20140304853; US20140298547; US20140295556; US20140294773; US20140287938; US20140273234; US20140273232; US20140273231; US20140273230; US20140271987; US20140256046; US20140248702; US20140242702; US20140242700; US20140242699; US20140242664; US20140234972; US20140227787; US20140212869; US20140201857; US20140199767; US20140189896; US20140186958; US20140186919; US20140186843; US20140179770; US20140179006; US20140170753; WO/2008/108989; WO/2010/054108; WO/2012/164565; WO/2013/098244; WO/2013/176772; US 20150071899; Makarova et al., “Evolution and classification of the CRISPR-Cas systems” 9(6) Nature Reviews Microbiology 467-477 (1-23) (June 2011); Wiedenheft et al., “RNA-guided genetic silencing systems in bacteria and archaea” 482 Nature 331-338 (Feb. 16, 2012); Gasiunas et al., “Cas9-crRNA ribonucleoprotein complex mediates specific DNA cleavage for adaptive immunity in bacteria” 109(39) Proceedings of the National Academy of Sciences USA E2579-E2586 (Sep. 4, 2012); Jinek et al., “A Programmable Dual-RNA-Guided DNA Endonuclease in Adaptive Bacterial Immunity” 337 Science 816-821 (Aug. 17, 2012); Carroll, “A CRISPR Approach to Gene Targeting” 20(9) Molecular Therapy 1658-1660 (September 2012); U.S. Appl. No. 61/652,086, filed May 25, 2012; Al-Attar et al., Clustered Regularly Interspaced Short Palindromic Repeats (CRISPRs): The Hallmark of an Ingenious Antiviral Defense Mechanism in Prokaryotes, Biol Chem. (2011) vol. 392, Issue 4, pp. 277-289; Hale et al., Essential Features and Rational Design of CRISPR RNAs That Function With the Cas RAMP Module Complex to Cleave RNAs, Molecular Cell, (2012) vol. 45, Issue 3, 292-302.

For methods in which the split deaminase base editors described herein, e.g., fusion proteins of the split deaminases, are delivered to cells, the proteins can be produced using any method known in the art, e.g., by in vitro translation, or expression in a suitable host cell from nucleic acid encoding the split deaminase, e.g., split deaminase fusion protein(s); a number of methods are known in the art for producing proteins. For example, the proteins can be produced in and purified from yeast, E. coli, insect cell lines, plants, transgenic animals, or cultured mammalian cells; see, e.g., Palomares et al., “Production of Recombinant Proteins: Challenges and Solutions,” Methods Mol Biol. 2004; 267:15-52. In addition, the split deaminases, e.g., split deaminase fusion protein(s), can be linked to a moiety that facilitates transfer into a cell, e.g., a lipid nanoparticle, optionally with a linker that is cleaved once the protein is inside the cell. See, e.g., LaFountaine et al., Int J Pharm. 2015 Aug. 13; 494(1):180-194.

### Methods of Use

The methods described herein include contacting cells with a nucleic acid encoding the split deaminase base editors described herein, e.g., fusion proteins of the split deaminases, and nucleic acids encoding one or more guide RNAs directed to a selected gene.

Guide RNAs (gRNAs)

Guide RNAs generally speaking come in two different systems: System 1, which uses separate crRNA and tracrRNAs that function together to guide cleavage by Cas9, and System 2, which uses a chimeric crRNA-tracrRNA hybrid that combines the two separate guide RNAs in a single system (referred to as a single guide RNA or sgRNA, see also Jinek et al., Science 2012; 337:816-821). The tracrRNA can be variably truncated and a range of lengths has been shown to function in both the separate system (system 1) and the chimeric gRNA system (system 2). For example, in some embodiments, tracrRNA may be truncated from its 3′ end by at least 1, 2, 3, 4, 5, 6, 7, 8, 9, 10, 15, 20, 25, 30, 35 or 40 nts. In some embodiments, the tracrRNA molecule may be truncated from its 5′ end by at least 1, 2, 3, 4, 5, 6, 7, 8, 9, 10, 15, 20, 25, 30, 35 or 40 nts. Alternatively, the tracrRNA molecule may be truncated from both the 5′ and 3′ end, e.g., by at least 1, 2, 3, 4, 5, 6, 7, 8, 9, 10, 15 or 20 nts on the 5′ end and at least 1, 2, 3, 4, 5, 6, 7, 8, 9, 10, 15, 20, 25, 30, 35 or 40 nts on the 3′ end. See, e.g., Jinek et al., Science 2012; 337:816-821; Mali et al., Science. 2013 Feb. 15; 339(6121):823-6; Cong et al., Science. 2013 Feb. 15; 339(6121):819-23; and Hwang and Fu et al., Nat Biotechnol. 2013 Mar.; 31(3):227-9; Jinek et al., Elife 2, e00471 (2013)). For System 2, generally the longer length chimeric gRNAs have shown greater on-target activity but the relative specificities of the various length gRNAs currently remain undefined and therefore it may be desirable in certain instances to use shorter gRNAs. In some embodiments, the gRNAs are complementary to a region that is within about 100-800 bp upstream of the transcription start site, e.g., is within about 500 bp upstream of the transcription start site, includes the transcription start site, or within about 100-800 bp, e.g., within about 500 bp, downstream of the transcription start site. In some embodiments, vectors (e.g., plasmids) encoding more than one gRNA are used, e.g., plasmids encoding, 2, 3, 4, 5, or more gRNAs directed to different sites in the same region of the target gene.

Cas9 nuclease can be guided to specific 17-20 nt genomic targets bearing an additional proximal protospacer adjacent motif (PAM), e.g., of sequence NGG, using a guide RNA, e.g., a single gRNA or a tracrRNA/crRNA, bearing 17-20 nts at its 5′ end that are complementary to the complementary strand of the genomic DNA target site. Thus, the present methods can include the use of a single guide RNA comprising a crRNA fused to a normally trans-encoded tracrRNA, e.g., a single Cas9 guide RNA as described in Mali et al., Science 2013 Feb. 15; 339(6121):823-6, with a sequence at the 5′ end that is complementary to the target sequence, e.g., of 25-17, optionally 20 or fewer nucleotides (nts), e.g., 20, 19, 18, or 17 nts, preferably 17 or 18 nts, of the complementary strand to a target sequence immediately 5′ of a protospacer adjacent motif (PAM), e.g., NGG, NAG, or NNGG. In some embodiments, the single Cas9 guide RNA consists of the sequence:

(SEQ ID NO: 51)
(X₁₇₋₂₀)GUUUUAGAGCUAGAAAUAGCAAGUUAAAAUAAGGCUAGUCCG
(X_{N});
(SEQ ID NO: 52)
(X₁₇₋₂₀)GUUUUAGAGCUAUGCUGAAAAGCAUAGCAAGUUAAAAUAAGG
CUAGUCCGUUAUC(X_{N});
(SEQ ID NO: 53)
(X₁₇₋₂₀)GUUUUAGAGCUAUGCUGUUUUGGAAACAAAACAGCAUAGCAA
GUUAAAAUAAGGCUAGUCCGUUAUC(X_{N});
(SEQ ID NO: 54)
(X₁₇₋₂₀)GUUUUAGAGCUAGAAAUAGCAAGUUAAAAUAAGGCUAGUCCG
UUAUCAACUUGAAAAAGUGGCACCGAGUCGGUGC(X_{N}),
(SEQ ID NO: 55)
(X₁₇₋₂₀)GUUUAAGAGCUAGAAAUAGCAAGUUUAAAUAAGGCUAGUCCG
UUAUCAACUUGAAAAAGUGGCACCGAGUCGGUGC;
(SEQ ID NO: 56)
(X₁₇₋₂₀)GUUUUAGAGCUAUGCUGGAAACAGCAUAGCAAGUUUAAAUAA
GGCUAGUCCGUUAUCAACUUGAAAAAGUGGCACCGAGUCGGUGC;
or
(SEQ ID NO: 57)
(X₁₇₋₂₀)GUUUAAGAGCUAUGCUGGAAACAGCAUAGCAAGUUUAAAUAA
GGCUAGUCCGUUAUCAACUUGAAAAAGUGGCACCGAGUCGGUGC;

wherein X₁₇₋₂₀ is the nucleotide sequence complementary to 17-20 consecutive nucleotides of the target sequence. DNAs encoding the single guide RNAs have been described previously in the literature (Jinek et al., Science. 337(6096):816-21 (2012) and Jinek et al., Elife. 2:e00471 (2013)).

The guide RNAs can include X_{N} which can be any sequence, wherein N (in the RNA) can be 0-200, e.g., 0-100, 0-50, or 0-20, that does not interfere with the binding of the ribonucleic acid to Cas9.

In some embodiments, the guide RNA includes one or more Adenine (A) or Uracil (U) nucleotides on the 3′ end. In some embodiments the RNA includes one or more U, e.g., 1 to 8 or more Us (e.g., U, UU, UUU, UUUU, UUUUU, UUUUUU, UUUUUUU, UUUUUUUU) at the 3′ end of the molecule, as a result of the optional presence of one or more Ts used as a termination signal to terminate RNA PolIII transcription.

Although some of the examples described herein utilize a single gRNA, the methods can also be used with dual gRNAs (e.g., the crRNA and tracrRNA found in naturally occurring systems). In this case, a single tracrRNA would be used in conjunction with multiple different crRNAs expressed using the present system, e.g., the following:

### (X₁₇₋₂₀) GUUUUAGAGCUA (SEQ ID NO:58);

### (X₁₇₋₂₀) GUUUUAGAGCUAUGCUGUUUUG (SEQ ID NO:59); or

(X₁₇₋₂₀) GUUUUAGAGCUAUGCU (SEQ ID NO:60); and a tracrRNA sequence. In this case, the crRNA is used as the guide RNA in the methods and molecules described herein, and the tracrRNA can be expressed from the same or a different DNA molecule. In some embodiments, the methods include contacting the cell with a tracrRNA comprising or consisting of the sequence GGAACCAUUCAAAACAGCAUAGCAAGUUAAAAUAAGGCUAGUCCGUUA UCAACUUGAAAAAGUGGCACCGAGUCGGUGC (SEQ ID NO:61) or an active portion thereof (an active portion is one that retains the ability to form complexes with Cas9 or dCas9). In some embodiments, the tracrRNA molecule may be truncated from its 3′ end by at least 1, 2, 3, 4, 5, 6, 7, 8, 9, 10, 15, 20, 25, 30, 35 or 40 nts. In another embodiment, the tracrRNA molecule may be truncated from its 5′ end by at least 1, 2, 3, 4, 5, 6, 7, 8, 9, 10, 15, 20, 25, 30, 35 or 40 nts. Alternatively, the tracrRNA molecule may be truncated from both the 5′ and 3′ end, e.g., by at least 1, 2, 3, 4, 5, 6, 7, 8, 9, 10, 15 or 20 nts on the 5′ end and at least 1, 2, 3, 4, 5, 6, 7, 8, 9, 10, 15, 20, 25, 30, 35 or 40 nts on the 3′ end. Exemplary tracrRNA sequences in addition to SEQ ID NO:8 include the following: UAGCAAGUUAAAAUAAGGCUAGUCCGUUAUCAACUUGAAAAAGUGGCA CCGAGUCGGUGC (SEQ ID NO:62) or an active portion thereof; or AGCAUAGCAAGUUAAAAUAAGGCUAGUCCGUUAUCAACUUGAAAAAGU GGCACCGAGUCGGUGC (SEQ ID NO:63) or an active portion thereof.

In some embodiments when (X₁₇₋₂₀) GUUUUAGAGCUAUGCUGUUUUG (SEQ ID NO:64) is used as a crRNA, the following tracrRNA is used: GGAACCAUUCAAAACAGCAUAGCAAGUUAAAAUAAGGCUAGUCCGUUA UCAACUUGAAAAAGUGGCACCGAGUCGGUGC (SEQ ID NO:65) or an active portion thereof.

In some embodiments when (X₁₇₋₂₀) GUUUUAGAGCUA (SEQ ID NO:66) is used as a crRNA, the following tracrRNA is used: UAGCAAGUUAAAAUAAGGCUAGUCCGUUAUCAACUUGAAAAAGUGGCA CCGAGUCGGUGC (SEQ ID NO:225) or an active portion thereof.

In some embodiments when (X₁₇₋₂₀) GUUUUAGAGCUAUGCU (SEQ ID NO:67) is used as a crRNA, the following tracrRNA is used: AGCAUAGCAAGUUAAAAUAAGGCUAGUCCGUUAUCAACUUGAAAAAGU GGCACCGAGUCGGUGC (SEQ ID NO:68) or an active portion thereof.

In some embodiments, the gRNA is targeted to a site that is at least three or more mismatches different from any sequence in the rest of the genome in order to minimize off-target effects.

Modified RNA oligonucleotides such as locked nucleic acids (LNAs) have been demonstrated to increase the specificity of RNA-DNA hybridization by locking the modified oligonucleotides in a more favorable (stable) conformation. For example, 2′-O-methyl RNA is a modified base where there is an additional covalent linkage between the 2′ oxygen and 4′ carbon which when incorporated into oligonucleotides can improve overall thermal stability and selectivity (Formula I).

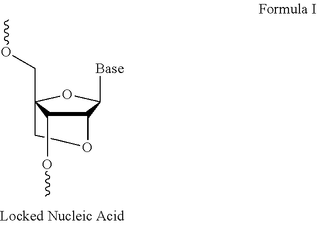

Thus in some embodiments, the tru-gRNAs disclosed herein may comprise one or more modified RNA oligonucleotides. For example, the truncated guide RNAs molecules described herein can have one, some or all of the region of the guideRNA complementary to the target sequence are modified, e.g., locked (2′-O-4′-C methylene bridge), 5′-methylcytidine, 2′-O-methyl-pseudouridine, or in which the ribose phosphate backbone has been replaced by a polyamide chain (peptide nucleic acid), e.g., a synthetic ribonucleic acid.

In other embodiments, one, some or all of the nucleotides of the tru-gRNA sequence may be modified, e.g., locked (2′-O-4′-C methylene bridge), 5′-methylcytidine, 2′-O-methyl-pseudouridine, or in which the ribose phosphate backbone has been replaced by a polyamide chain (peptide nucleic acid), e.g., a synthetic ribonucleic acid.

In some embodiments, the single guide RNAs and/or crRNAs and/or tracrRNAs can include one or more Adenine (A) or Uracil (U) nucleotides on the 3′ end.

Existing Cas9-based RGNs use gRNA-DNA heteroduplex formation to guide targeting to genomic sites of interest. However, RNA-DNA heteroduplexes can form a more promiscuous range of structures than their DNA-DNA counterparts. In effect, DNA-DNA duplexes are more sensitive to mismatches, suggesting that a DNA-guided nuclease may not bind as readily to off-target sequences, making them comparatively more specific than RNA-guided nucleases. Thus, the guide RNAs usable in the methods described herein can be hybrids, i.e., wherein one or more deoxyribonucleotides, e.g., a short DNA oligonucleotide, replaces all or part of the gRNA, e.g., all or part of the complementarity region of a gRNA. This DNA-based molecule could replace either all or part of the gRNA in a single gRNA system or alternatively might replace all of part of the crRNA and/or tracrRNA in a dual crRNA/tracrRNA system. Such a system that incorporates DNA into the complementarity region should more reliably target the intended genomic DNA sequences due to the general intolerance of DNA-DNA duplexes to mismatching compared to RNA-DNA duplexes. Methods for making such duplexes are known in the art, See, e.g., Barker et al., BMC Genomics. 2005 Apr. 22; 6:57; and Sugimoto et al., Biochemistry. 2000 Sep. 19; 39(37):11270-81.

In addition, in a system that uses separate crRNA and tracrRNA, one or both can be synthetic and include one or more modified (e.g., locked) nucleotides or deoxyribonucleotides.

In a cellular context, complexes of Cas9 with these synthetic gRNAs could be used to improve the genome-wide specificity of the CRISPR/Cas9 nuclease system.

The methods described can include expressing in a cell, or contacting the cell with, a Cas9 gRNA plus a fusion protein as described herein.

Expression Systems

To use the split deaminase base editors described herein, e.g., fusion proteins of the split deaminases, it may be desirable to express them from a nucleic acid that encodes them. This can be performed in a variety of ways. For example, the nucleic acid encoding the split deaminase fusion can be cloned into an intermediate vector for transformation into prokaryotic or eukaryotic cells for replication and/or expression. Intermediate vectors are typically prokaryote vectors, e.g., plasmids, or shuttle vectors, or insect vectors, for storage or manipulation of the nucleic acid encoding the split deaminase fusion for production of the split deaminase, e.g., split deaminase fusion protein(s). The nucleic acid encoding the split deaminase, e.g., split deaminase fusion protein(s), can also be cloned into an expression vector, for administration to a plant cell, animal cell, preferably a mammalian cell or a human cell, fungal cell, bacterial cell, or protozoan cell.

To obtain expression, a sequence encoding a split deaminase base editor, e.g., fusion protein(s) of the split deaminases, is typically subcloned into an expression vector that contains a promoter to direct transcription. Suitable bacterial and eukaryotic promoters are well known in the art and described, e.g., in Sambrook et al., Molecular Cloning, A Laboratory Manual (3d ed. 2001); Kriegler, Gene Transfer and Expression: A Laboratory Manual (1990); and Current Protocols in Molecular Biology (Ausubel et al., eds., 2010). Bacterial expression systems for expressing the engineered protein are available in, e.g., E. coli, Bacillus sp., and Salmonella (Palva et al., 1983, Gene 22:229-235). Kits for such expression systems are commercially available. Eukaryotic expression systems for mammalian cells, yeast, and insect cells are well known in the art and are also commercially available.

The promoter used to direct expression of a nucleic acid depends on the particular application. For example, a strong constitutive promoter is typically used for expression and purification of fusion proteins. In contrast, when the split deaminase, e.g., split deaminase fusion protein(s), is to be administered in vivo for gene regulation, either a constitutive or an inducible promoter can be used, depending on the particular use of the split deaminase, e.g., split deaminase fusion protein(s). In addition, a preferred promoter for administration of the split deaminase, e.g., split deaminase fusion protein(s), can be a weak promoter, such as HSV TK or a promoter having similar activity. The promoter can also include elements that are responsive to transactivation, e.g., hypoxia response elements, Gal4 response elements, lac repressor response element, and small molecule control systems such as tetracycline-regulated systems and the RU-486 system (see, e.g., Gossen & Bujard, 1992, Proc. Natl. Acad. Sci. USA, 89:5547; Oligino et al., 1998, Gene Ther., 5:491-496; Wang et al., 1997, Gene Ther., 4:432-441; Neering et al., 1996, Blood, 88:1147-55; and Rendahl et al., 1998, Nat. Biotechnol., 16:757-761).

In addition to the promoter, the expression vector typically contains a transcription unit or expression cassette that contains all the additional elements required for the expression of the nucleic acid in host cells, either prokaryotic or eukaryotic. Atypical expression cassette thus contains a promoter operably linked, e.g., to the nucleic acid sequence encoding the split deaminase, e.g., split deaminase fusion protein(s), and any signals required, e.g., for efficient polyadenylation of the transcript, transcriptional termination, ribosome binding sites, or translation termination. Additional elements of the cassette may include, e.g., enhancers, and heterologous spliced intronic signals.

The particular expression vector used to transport the genetic information into the cell is selected with regard to the intended use of the fusion protein, e.g., expression in plants, animals, bacteria, fungus, protozoa, etc. Standard bacterial expression vectors include plasmids such as pBR322 based plasmids, pSKF, pET23D, and commercially available tag-fusion expression systems such as GST and LacZ. A preferred tag-fusion protein is the maltose binding protein (MBP). Such tag-fusion proteins can be used for purification of the engineered TALE repeat protein. Epitope tags can also be added to recombinant proteins to provide convenient methods of isolation, for monitoring expression, and for monitoring cellular and subcellular localization, e.g., c-myc or FLAG.

Expression vectors containing regulatory elements from eukaryotic viruses are often used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 late promoter, metallothionein promoter, murine mammary tumor virus promoter, Rous sarcoma virus promoter, polyhedrin promoter, or other promoters shown effective for expression in eukaryotic cells.

The vectors for expressing the guide RNAs can include RNA Pol III promoters to drive expression of the guide RNAs, e.g., the H1, U6 or 7SK promoters. These human promoters allow for expression of split deaminase, e.g., split deaminase fusion protein(s), in mammalian cells following plasmid transfection. Alternatively, a T7 promoter may be used, e.g., for in vitro transcription, and the RNA can be transcribed in vitro and purified. Vectors suitable for the expression of short RNAs, e.g., siRNAs, shRNAs, or other small RNAs, can be used.

Some expression systems have markers for selection of stably transfected cell lines such as thymidine kinase, hygromycin B phosphotransferase, and dihydrofolate reductase. High yield expression systems are also suitable, such as using a baculovirus vector in insect cells, with the gRNA encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of recombinant sequences.

Standard transfection methods are used to produce bacterial, mammalian, yeast or insect cell lines that express large quantities of protein, which are then purified using standard techniques (see, e.g., Colley et al., 1989, J. Biol. Chem., 264:17619-22; Guide to Protein Purification, in Methods in Enzymology, vol. 182 (Deutscher, ed., 1990)). Transformation of eukaryotic and prokaryotic cells are performed according to standard techniques (see, e.g., Morrison, 1977, J. Bacteriol. 132:349-351; Clark-Curtiss & Curtiss, Methods in Enzymology 101:347-362 (Wu et al., eds, 1983).

Any of the known procedures for introducing foreign nucleotide sequences into host cells may be used. These include the use of calcium phosphate transfection, polybrene, protoplast fusion, electroporation, nucleofection, liposomes, microinjection, naked DNA, plasmid vectors, viral vectors, both episomal and integrative, and any of the other well-known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the split deaminase, e.g., split deaminase fusion protein(s).

In some embodiments, the fusion protein includes a nuclear localization domain which provides for the protein to be translocated to the nucleus. Several nuclear localization sequences (NLS) are known, and any suitable NLS can be used. For example, many NLSs have a plurality of basic amino acids, referred to as a bipartite basic repeats (reviewed in Garcia-Bustos et al, 1991, Biochim. Biophys. Acta, 1071:83-101). An NLS containing bipartite basic repeats can be placed in any portion of chimeric protein and results in the chimeric protein being localized inside the nucleus. In preferred embodiments a nuclear localization domain is incorporated into the final fusion protein, as the ultimate functions of the fusion proteins described herein will typically require the proteins to be localized in the nucleus. However, it may not be necessary to add a separate nuclear localization domain in cases where the DBD domain itself, or another functional domain within the final chimeric protein, has intrinsic nuclear translocation function.

In methods wherein the fusion proteins include a Cas9 domain, the methods also include delivering a gRNA that interacts with the Cas9.

Alternatively, the methods can include delivering the split deaminase, e.g., split deaminase fusion protein(s), and guide RNA together, e.g., as a complex. For example, the split deaminase, e.g., split deaminase fusion protein(s), and gRNA can be can be overexpressed in a host cell and purified, then complexed with the guide RNA (e.g., in a test tube) to form a ribonucleoprotein (RNP), and delivered to cells. In some embodiments, the split deaminase, e.g., split deaminase fusion protein(s), can be expressed in and purified from bacteria through the use of bacterial expression plasmids. For example, His-tagged split deaminase, e.g., split deaminase fusion protein(s), can be expressed in bacterial cells and then purified using nickel affinity chromatography. The use of RNPs circumvents the necessity of delivering plasmid DNAs encoding the nuclease or the guide, or encoding the nuclease as an mRNA. RNP delivery may also improve specificity, presumably because the half-life of the RNP is shorter and there's no persistent expression of the nuclease and guide (as you'd get from a plasmid). The RNPs can be delivered to the cells in vivo or in vitro, e.g., using lipid-mediated transfection or electroporation. See, e.g., Liang et al. “Rapid and highly efficient mammalian cell engineering via Cas9 protein transfection.” Journal of biotechnology 208 (2015): 44-53; Zuris, John A., et al. “Cationic lipid-mediated delivery of proteins enables efficient protein-based genome editing in vitro and in vivo.” Nature biotechnology 33.1 (2015): 73-80; Kim et al. “Highly efficient RNA-guided genome editing in human cells via delivery of purified Cas9 ribonucleoproteins.” Genome research 24.6 (2014): 1012-1019.

The present invention also includes the vectors and cells comprising the vectors, as well as kits comprising the proteins and nucleic acids described herein, e.g., for use in a method described herein.

### Examples

The invention is further described in the following examples, which do not limit the scope of the invention described in the claims.

### Example 1: Materials and Methods

Various split base editing enzymes were engineered as described in Examples 2-7. Both on-site and off-site editing rates for the various enzymes was measured as described herein. gRNA sequences by target site are shown in Table 4.

TABLE 4
Target Site Sequences

| Target Site | gRNA Sequence | SEQ ID NO: |
|---|---|---|
| EMX1-1 | GAGTCCGAGCAGAAGAAGAA | 27 |
| EMX1 -2 | GTATTCACCTGAAAGTGTGC | 28 |
| FANCF | GGAATCCCTTCTGCAGCACC | 29 |
| HEK Site 2 | GAACACAAAGCATAGACTGC | 30 |
| HEK Site 3 | GGCCCAGACTGAGCACGTGA | 31 |
| HEK Site 4 | GGCACTGCGGCTGGAGGTGG | 32 |
| PDCD1 | GCGTGACTTCCACATGAGCG | 33 |
| PPP1R12C 1 | GACTCACCCAGGAGTGCGTT | 34 |
| PPP1R12C 2 | GGCACTCGGGGGCGAGAGGA | 35 |
| PPP1R12C 3 | GAGCTCACTGAACGCTGGCA | 36 |
| RNF2 | GTCATCTTAGTCATTACCTG | 37 |
| VEGFA | GACCCCCTCCACCCCGCCTC | 38 |

### Example 2: Engineering of sDA-BE

We developed a split deaminase base editor (sDA-BE) by bisecting the BE4-Max (SEQ ID NO:1) (see also FIG. 106 and SEQ ID NO:s 70-72) into two cognate pieces, the first of which spans from residues 1-90 (sDA1.2) (SEQ ID NO:2) and the other from residues 100-1854 of BE4-Max, with the additional of a methionine at the N-terminus (sDA2.2) (SEQ ID NO:3). Importantly, this architecture conserved the nuclear localization signal (NLS) (SEQ ID NO:4) present at the N and C termini of BE4, resulting in one bipartite NLS each for sDA1.2 and sDA2.2, whereas BE4 contains two.

BE4-Max
(SEQ ID NO: 1)
MKRTADGSEFESPKKKRKVSSETGPVAVDPTLRRRIEPHEFEVFFDPRELRKETCLL
YEINWGGRHSIWRHTSQNTNKHVEVNFIEKFTTERYFCPNTRCSITWFLSWSPCGEC
SRAITEFLSRYPHVTLFIYIARLYHHADPRNRQGLRDLISSGVTIQIMTEQESGYCW
RNFVNYSPSNEAHWPRYPHLWVRLYVLELYCIILGLPPCLNILRRKQPQLTFFTIAL
QSCHYQRLPPHILWATGLKSGGSSGGSSGSETPGTSESATPESSGGSSGGSDKKYSI
GLAIGTNSVGWAVITDEYKVPSKKFKVLGNTDRHSIKKNLIGALLFDSGETAEATRL
KRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDN
SDVDKLFIQLVQTYNQLFEENPINASGVDAKAILSARLSKSRRLENLIAQLPGEKKN
GLFGNLIALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQLPEKYKE
IFFDQSKNGYAGYIDGGASQEEFYKFIKPILEKMDGTEELLVKLNREDLLRKQRTFD
NGSIPHQIHLGELHAILRRQEDFYPFLKDNREKIEKILTFRIPYYVGPLARGNSRFA
WMTRKSEETITPWNFEEVVDKGASAQSFIERMTNFDKNLPNEKVLPKHSLLYEYFTV
YNELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFANR
NFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQ
LQNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIKR
QLVETRQITKHVAQILDSRMNTKYDENDKLIREVKVITLKSKLVSDFRKDFQFYKVR
EINNYHHAHDAYLNAVVGTALIKKYPKLESEFVYGDYKVYDVRKMIAKSEQEIGKAT
AKYFFYSNIMNFFKTEITLANGEIRKRPLIETNGETGEIVWDKGRDFATVRKVLSMP
QVNIVKKTEVQTGGFSKESILPKRNSDKLIARKKDWDPKKYGGFDSPTVAYSVLVVA
KVEKGKSKKLKSVKELLGITIMERSSFEKNPIDFLEAKGYKEVKKDLIIKLPKYSLF
ELENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNL
GAPAAFKYFDTTIDRKRYTSTKEVLDATLIHQSITGLYETRIDLSQLGGDSGGSGGS
GGSTNLSDIIEKETGKQLVIQESILMLPEEVEEVIGNKPESDILVHTAYDESTDENV
MLLTSDAPEYKPWALVIQDSNGENKIKMLSGGSGGSGGSTNLSDIIEKETGKQLVIQ
ESILMLPEEVEEVIGNKPESDILVHTAYDESTDENVMLLTSDAPEYKPWALVIQDSN
GENKIKMLSGGSKRTADGSEFEPKKKRKV
sDA 1.2
(SEQ ID NO: 2)
MKRTADGSEFESPKKKRKVSSETGPVAVDPTLRRRIEPHEFEVFFDPRELRKETCLL
YEINWGGRHSIWRHTSQNTNKHVEVNFIEKFTT
sDA 2.2
(SEQ ID NO: 3)
MCSITWFLSWSPCGECSRAITEFLSRYPHVTLFIYIARLYHHADPRNRQGLRDLISS
GVTIQIMTEQESGYCWRNFVNYSPSNEAHWPRYPHLWVRLYVLELYCIILGLPPCLN
ILRRKQPQLTFFTIALQSCHYQRLPPHILWATGLKSGGSSGGSSGSETPGTSESATP
ESSGGSSGGSDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESF
LVEEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLSKS
RRLENLIAQLPGEKKNGLFGNLIALSLGLTPNFKSNFDLAEDAKLQLSKDTYDDDLD
NLLAQIGDQYADLFLAAKNLSDAILLSDILRVNTEITKAPLSASMIKRYDEHHQDLT
LLKALVRQQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKIEKILTFR
IPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEENEDILED
IVLTLTLFEDREMIEERLKTYAHLFDDKVMKQLKRRRYTGWGRLSRKLINGIRDKQS
GKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIK
ELGSQILKEHPVENTQLQNEKLYLYYLQNGRDMYVDQELDINRLSDYDVDHIVPQSF
LKDDSIDNKVLTRSDKNRGKSDNVPSEEVVKKMKNYWRQLLNAKLITQRKFDNLTKA
ERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYD
VRKMIAKSEQEIGKATAKYFFYSNIMNFFKTEITLANGEIRKRPLIETNGETGEIVW
DKGRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKGYK
EVKKDLIIKLPKYSLFELENGRKRMLASAGELQKGNELALPSKYVNFLYLASHYEKL
KGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETR
IDLSQLGGDSGGSGGSGGSTNLSDIIEKETGKQLVIQESILMLPEEVEEVIGNKPES
DILVHTAYDESTDENVMLLTSDAPEYKPWALVIQDSNGENKIKMLSGGSGGSGGSTN
LSDIIEKETGKQLVIQESILMLPEEVEEVIGNKPESDILVHTAYDESTDENVMLLTS
DAPEYKPWALVIQDSNGENKIKMLSGGSKRTADGSEFEPKKKRKV
N-Terminal Bipartite NLS (BPNLS)
(SEQ ID NO: 4)
MKRTADGSEFESPKKKRKV

### Example 3: Variants of sDA-BE

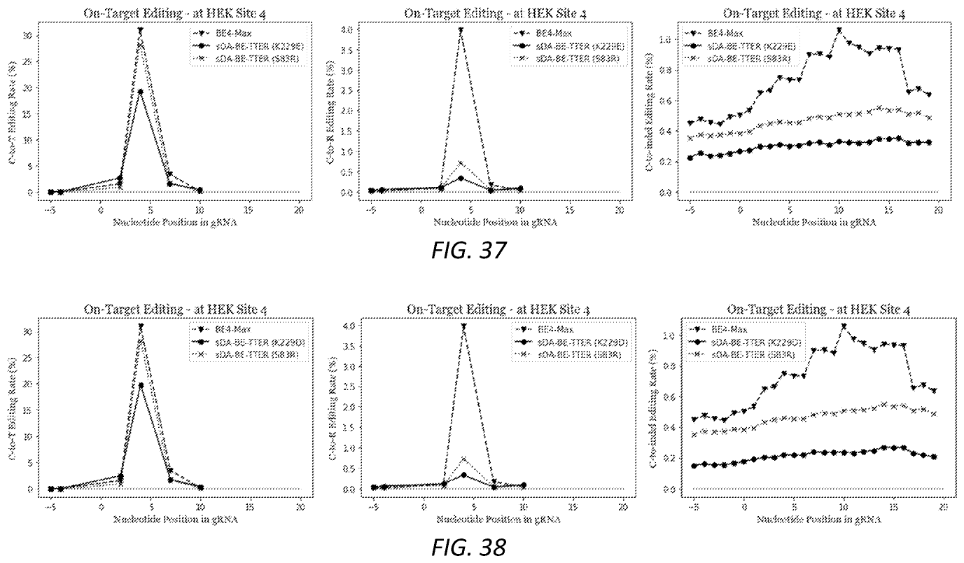

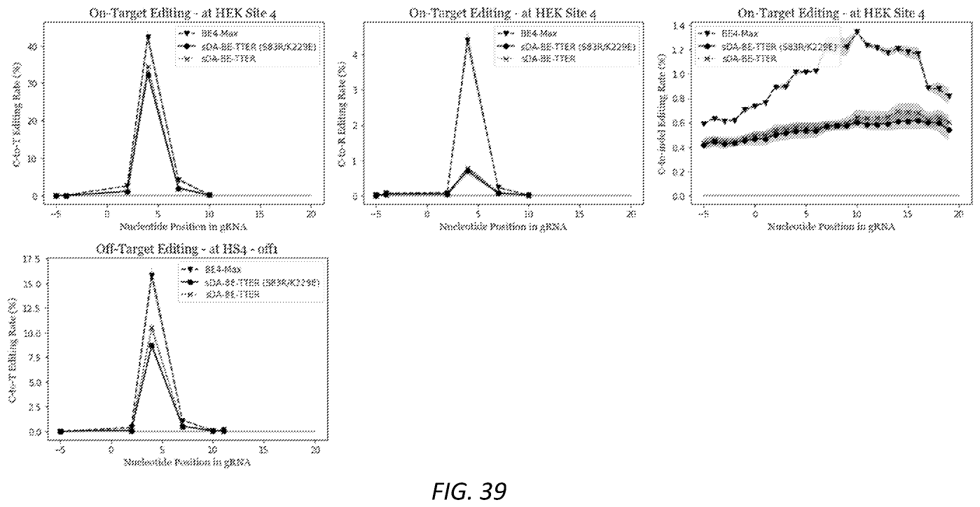

We further engineered the sDA-BE to substantially increase the editing rates, approaching that of the highly-active BE3 and BE4-Max. We found that extending the N-terminus of the sDA2.2 to include 3 more residues of BE4-Max (97-99) and converting the residue corresponding to the Ser-83 of the rAPOBEC1 (corresponding to S101R in BE4-Max) to an arginine (SEQ ID NO:5) (sDA-BE S83R) or the residue corresponding to the Lys-229 of the rAPOBEC (corresponding to K247E in BE4-Max) to glutamic acid or aspartic acid (SEQ ID NOs: 6 and 7, respectively) (sDA-BE K229E and sDA-BE K229D, respectively) (where N97 is preceded by Met-Gly diresidue to create an optimal Kozak sequence) facilitates efficient editing without the need to localize sDA1.2 using a second DNA binding domain (such as a zinc-finger) as we expected previously. sDA-BE-K229E and sDA-BE-K229D show improved editing over wild-type sDA2.2 but not over sDA-BE-S83R (FIGS. 37-39). While the exact mechanism of action of these modifications is unknown, we presume that they affect the binding affinity of the interaction between the two sDA modules. For example, S83R of rAPOBEC1 on sDA2.2 could potentially create a salt bridge with the E41 residue of the rAPOBEC1 domain on sDA1.2, possibly facilitating an interaction between the two sDA-BE modules. Therefore, we predict that similar mutations (such as S83K or S83H) that affect binding interactions between the sDA-BE pieces may result in similar outcomes.

sDA2.2-BE S83R
(SEQ ID NO: 5)
MGNTRCRITWFLSWSPCGECSRAITEFLSRYPHVTLFIYIARLYHHADPRNRQGLRD
LISSGVTIQIMTEQESGYCWRNFVNYSPSNEAHWPRYPHLWVRLYVLELYCIILGLP
PCLNILRRKQPQLTFFTIALQSCHYQRLPPHILWATGLKSGGSSGGSSGSETPGTSE
SATPESSGGSSGGSDKKYSIGLAIGTNSVGWAVITDEYKVPSKKFKVLGNTDRHSIK
KNLIGALLFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADLRLIYLAL
AHMIKFRGHFLIEGDLNPDNSDVDKLFIQLVQTYNQLFEENPINASGVDAKAILSAR
LSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHH
QDLTLLKALVRQQLPEKYKEIFFDQSKNGYAGYIDGGASQEEFYKFIKPILEKMDGT
EELLVKLNREDLLRKQRTFDNGSIPHQIHLGELHAILRRQEDFYPFLKDNREKIEKI
LTFRIPYYVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEENED
ILEDIVLTLTLFEDREMIEERLKTYAHLFDDKVMKQLKRRRYTGWGRLSRKLINGIR
DKQSGKTILDFLKSDGFANRNFMQLIHDDSLTFKEDIQKAQVSGQGDSLHEHIANLA
GSPAIKKGILQTVKVVDELVKVMGRHKPENIVIEMARENQTTQKGQKNSRERMKRIE
EGIKELGSQILKEHPVENTQLQNEKLYLYYLQNGRDMYVDQELDINRLSDYDVDHIV
PQSFLKDDSIDNKVLTRSDKNRGKSDNVPSEEVVKKMKNYWRQLLNAKLITQRKFDN
LTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETG
EIVWDKGRDFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVNFLYLASH
YEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGL
YETRIDLSQLGGDSGGSGGSGGSTNLSDIIEKETGKQLVIQESILMLPEEVEEVIGN
KPESDILVHTAYDESTDENVMLLTSDAPEYKPWALVIQDSNGENKIKMLSGGSGGSG
GSTNLSDIIEKETGKQLVIQESILMLPEEVEEVIGNKPESDILVHTAYDESTDENVM
LLTSDAPEYKPWALVIQDSNGENKIKMLSGGSKRTADGSEFEPKKKRKV
sDA2.2-BE K229E
(SEQ ID NO: 6)
MGNTRCSITWFLSWSPCGECSRAITEFLSRYPHVTLFIYIARLYHHADPRNRQGLRD
LISSGVTIQIMTEQESGYCWRNFVNYSPSNEAHWPRYPHLWVRLYVLELYCIILGLP
PCLNILRRKQPQLTFFTIALQSCHYQRLPPHILWATGLESGGSSGGSSGSETPGTSE
SATPESSGGSSGGSDKKYSIGLAIGTNSVGWAVITDEYKVPSKKFKVLGNTDRHSIK
KNLIGALLFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADLRLIYLAL
AHMIKFRGHFLIEGDLNPDNSDVDKLFIQLVQTYNQLFEENPINASGVDAKAILSAR
LSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHH
QDLTLLKALVRQQLPEKYKEIFFDQSKNGYAGYIDGGASQEEFYKFIKPILEKMDGT
EELLVKLNREDLLRKQRTFDNGSIPHQIHLGELHAILRRQEDFYPFLKDNREKIEKI
LTFRIPYYVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEENED
ILEDIVLTLTLFEDREMIEERLKTYAHLFDDKVMKQLKRRRYTGWGRLSRKLINGIR
DKQSGKTILDFLKSDGFANRNFMQLIHDDSLTFKEDIQKAQVSGQGDSLHEHIANLA
GSPAIKKGILQTVKVVDELVKVMGRHKPENIVIEMARENQTTQKGQKNSRERMKRIE
EGIKELGSQILKEHPVENTQLQNEKLYLYYLQNGRDMYVDQELDINRLSDYDVDHIV
PQSFLKDDSIDNKVLTRSDKNRGKSDNVPSEEVVKKMKNYWRQLLNAKLITQRKFDN
LTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETG
EIVWDKGRDFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVNELYLASH
YEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGL
YETRIDLSQLGGDSGGSGGSGGSTNLSDIIEKETGKQLVIQESILMLPEEVEEVIGN
KPESDILVHTAYDESTDENVMLLTSDAPEYKPWALVIQDSNGENKIKMLSGGSGGSG
GSTNLSDIIEKETGKQLVIQESILMLPEEVEEVIGNKPESDILVHTAYDESTDENVM
LLTSDAPEYKPWALVIQDSNGENKIKMLSGGSKRTADGSEFEPKKKRKV
sDA2.2-BE K229D
(SEQ ID NO: 7)
MGNTRCSITWFLSWSPCGECSRAITEFLSRYPHVTLFIYIARLYHHADPRNRQGLRD
LISSGVTIQIMTEQESGYCWRNFVNYSPSNEAHWPRYPHLWVRLYVLELYCIILGLP
PCLNILRRKQPQLTFFTIALQSCHYQRLPPHILWATGLDSGGSSGGSSGSETPGTSE
SATPESSGGSSGGSDKKYSIGLAIGTNSVGWAVITDEYKVPSKKFKVLGNTDRHSIK
KNLIGALLFDSGETAEATRLKRTARRRYTRRKNRICYLQEIFSNEMAKVDDSFFHRL
EESFLVEEDKKHERHPIFGNIVDEVAYHEKYPTIYHLRKKLVDSTDKADLRLIYLAL
AHMIKFRGHFLIEGDLNPDNSDVDKLFIQLVQTYNQLFEENPINASGVDAKAILSAR
LSKSRRLENLIAQLPGEKKNGLFGNLIALSLGLTPNFKSNFDLAEDAKLQLSKDTYD
DDLDNLLAQIGDQYADLFLAAKNLSDAILLSDILRVNTEITKAPLSASMIKRYDEHH
QDLTLLKALVRQQLPEKYKEIFFDQSKNGYAGYIDGGASQEEFYKFIKPILEKMDGT
EELLVKLNREDLLRKQRTFDNGSIPHQIHLGELHAILRRQEDFYPFLKDNREKIEKI
LTFRIPYYVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVDLLFKTNR
KVTVKQLKEDYFKKIECFDSVEISGVEDRFNASLGTYHDLLKIIKDKDFLDNEENED
ILEDIVLTLTLFEDREMIEERLKTYAHLFDDKVMKQLKRRRYTGWGRLSRKLINGIR
DKQSGKTILDFLKSDGFANRNFMQLIHDDSLTFKEDIQKAQVSGQGDSLHEHIANLA
GSPAIKKGILQTVKVVDELVKVMGRHKPENIVIEMARENQTTQKGQKNSRERMKRIE
EGIKELGSQILKEHPVENTQLQNEKLYLYYLQNGRDMYVDQELDINRLSDYDVDHIV
PQSFLKDDSIDNKVLTRSDKNRGKSDNVPSEEVVKKMKNYWRQLLNAKLITQRKFDN
LTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDY
KVYDVRKMIAKSEQEIGKATAKYFFYSNIMNFFKTEITLANGEIRKRPLIETNGETG
EIVWDKGRDFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVNELYLASH
YEKLKGSPEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLIHQSITGL
YETRIDLSQLGGDSGGSGGSGGSTNLSDIIEKETGKQLVIQESILMLPEEVEEVIGN
KPESDILVHTAYDESTDENVMLLTSDAPEYKPWALVIQDSNGENKIKMLSGGSGGSG
GSTNLSDIIEKETGKQLVIQESILMLPEEVEEVIGNKPESDILVHTAYDESTDENVM
LLTSDAPEYKPWALVIQDSNGENKIKMLSGGSKRTADGSEFEPKKKRKV

### Example 4: sDA-BE-TT and sDA-BE-TTER

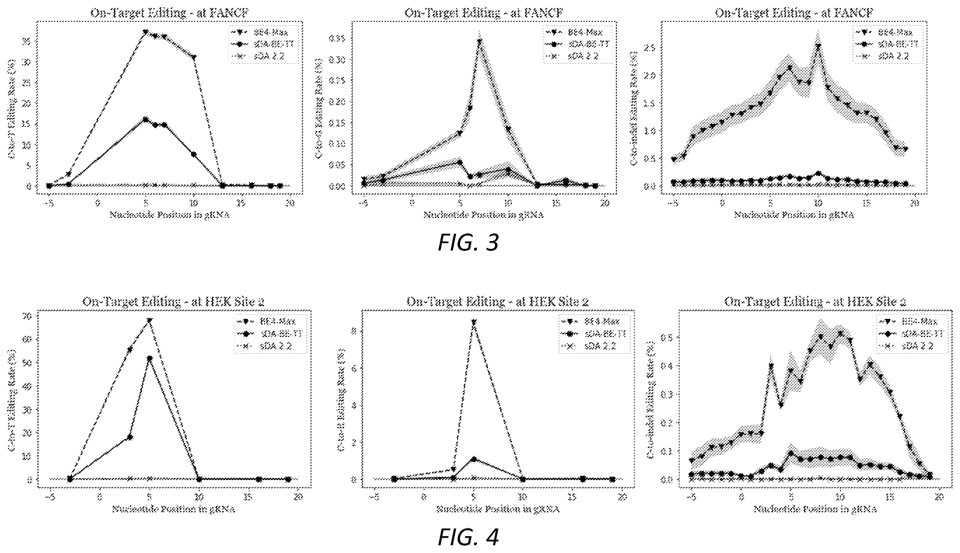

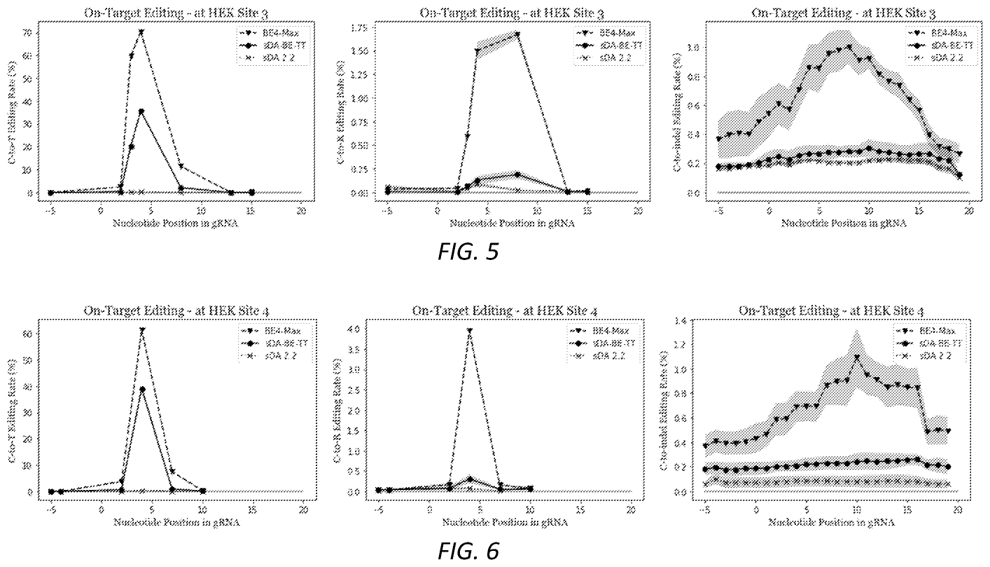

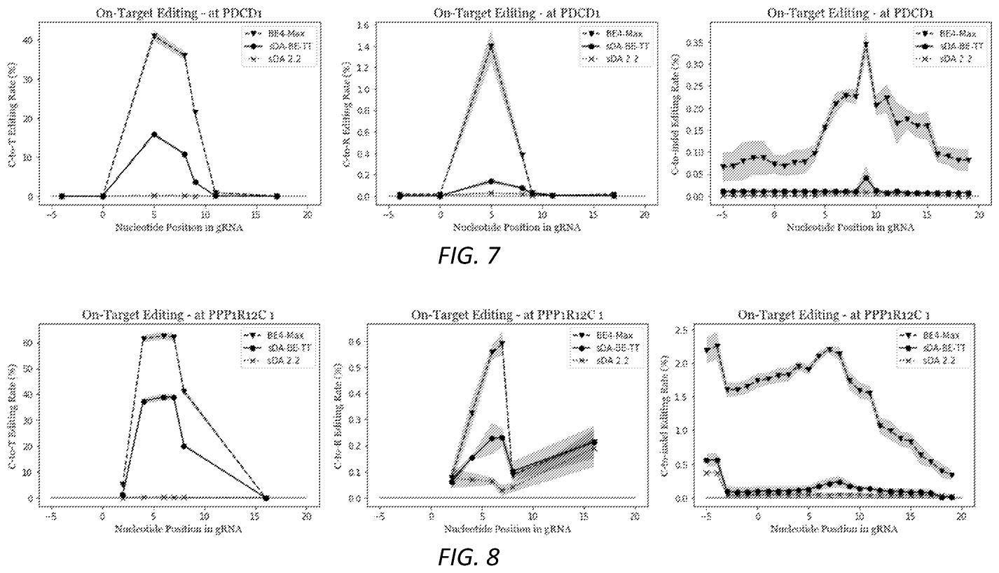

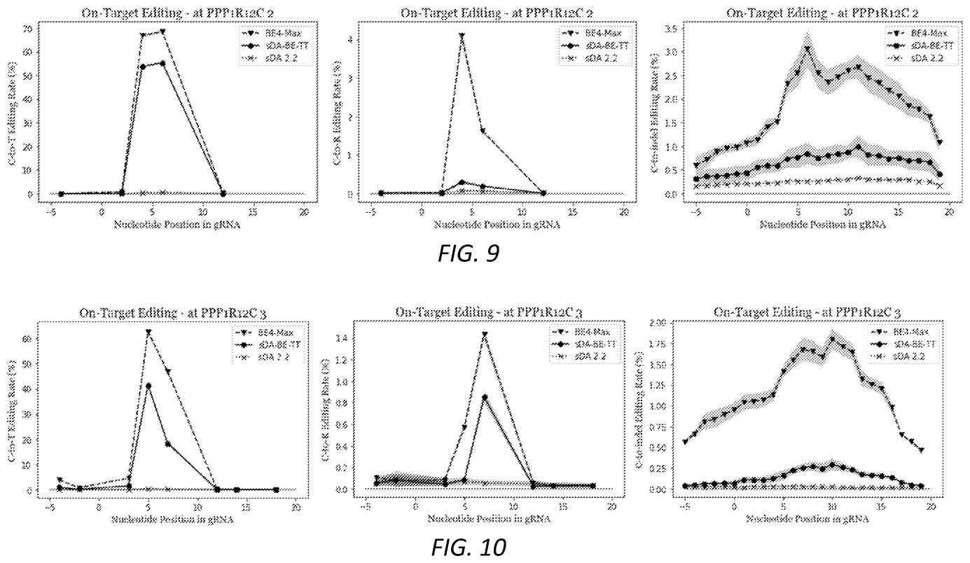

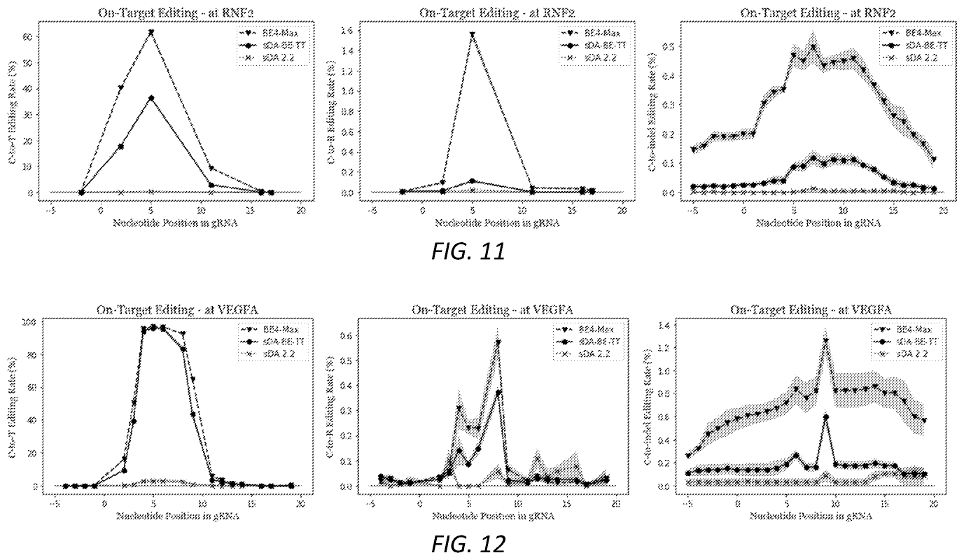

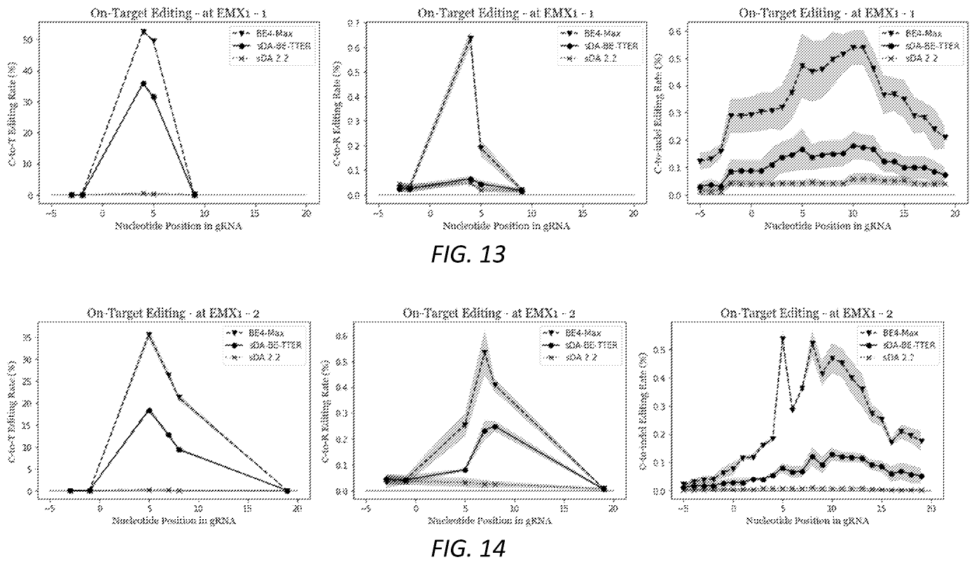

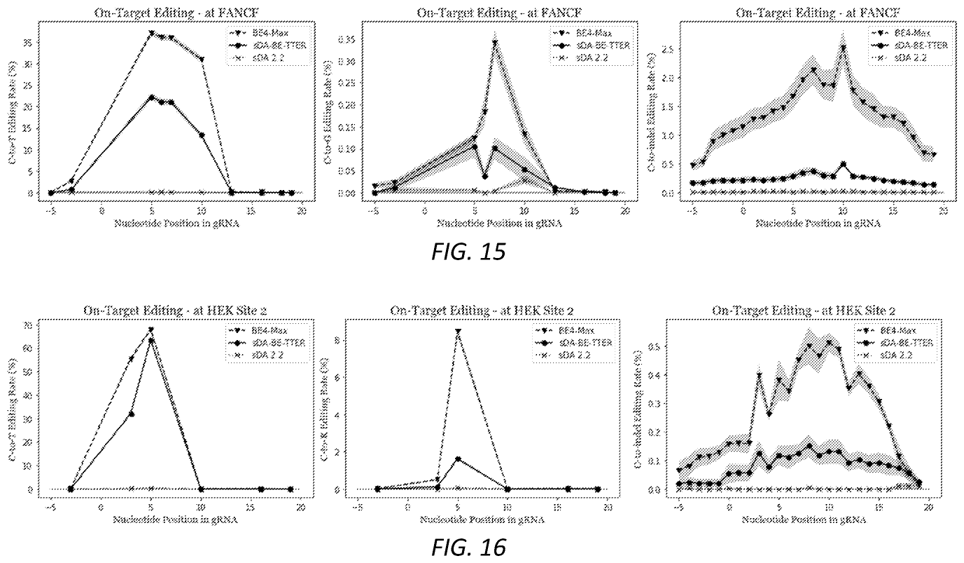

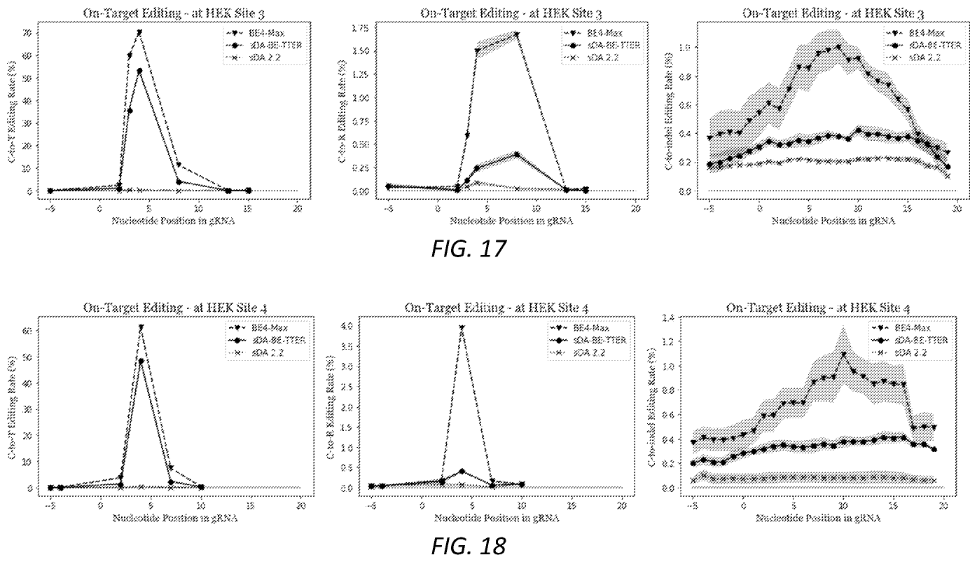

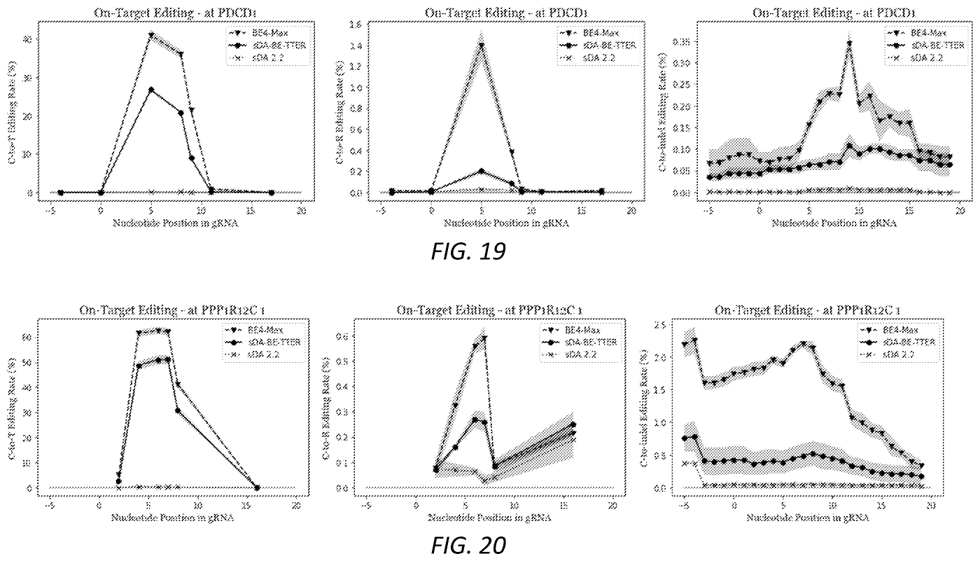

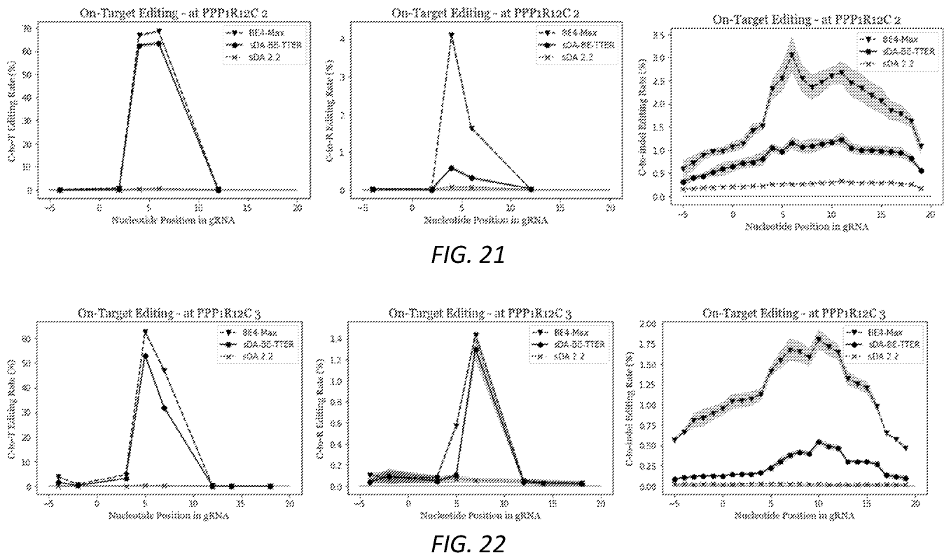

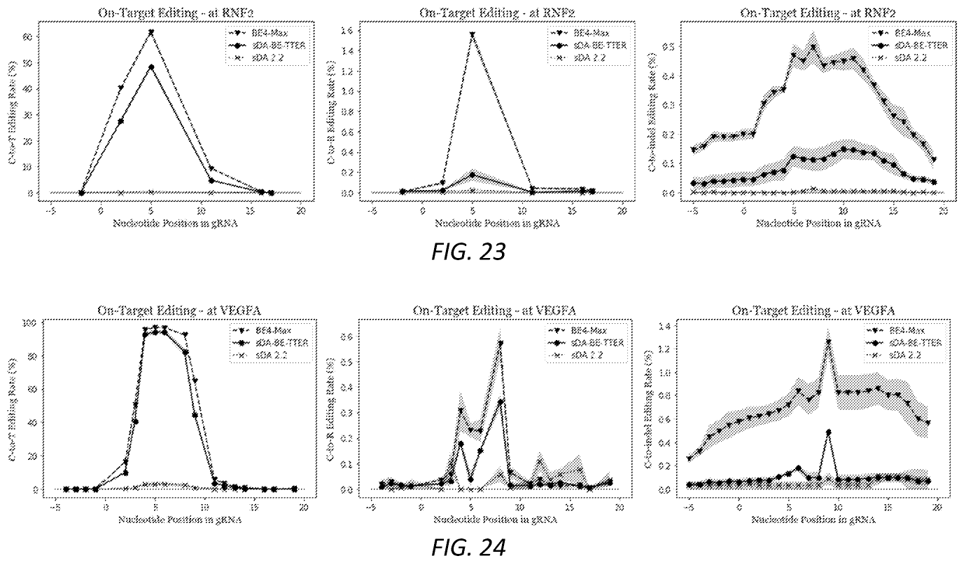

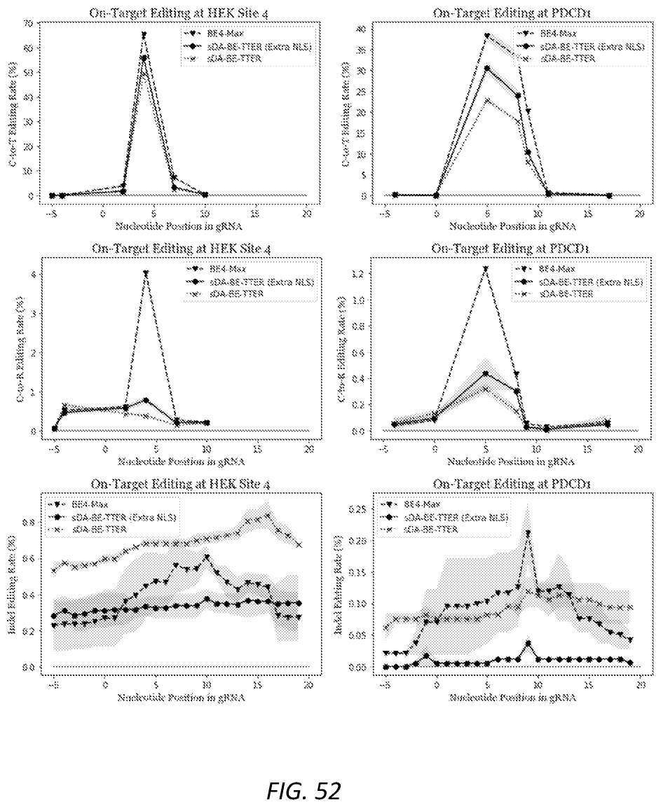

Extending the C-terminus of the sDA1.2 domain from T90 of BE4-Max to E91 (SEQ ID NO:8) or R92 (resulting in a domain ending in the amino acid sequence -TTER) (SEQ ID NO:9) further improved the activity of S83R-containing sDA-BEs, and would presumably have a similar effect if used in sDA-BEs lacking the S83R mutation as well. With the exception of K229E or K229D, the effects of which seem to improve editing over wild-type sDA2.2 but not over S83R sDA2.2, the highest sDA-BE editing rates can be achieved by an editor with an sDA1.2 comprising residues 1-92 of BE4-Max (SEQ ID NO:9) and an sDA2.2 comprising residues 97-99 of BE4-Max, (where N97 is preceded by Met-Gly diresidue to create an optimal Kozak sequence) with the S83R mutation (SEQ ID NO:5). We designated this editor sDA-BE-TTER. We term a similar editor comprising the same sDA2.2 module as above but a sDA1.2 module with residues 1-90 of BE4-Max with a T at 90 (SEQ ID NO:10) as sDA-BE-TT. The interaction between the sDA1.2 and sDA2.2 modules appears strong enough to support substantial on-target editing without the need to localize the sDA1.2. Of note, editing by both sDA-BE-TT and sDA-BE-TTER results in lower rates of cytosine-to-purine (C-to-R) edits and indel formation than BE4-Max. Data showing C-to-T, C-to-R, and indel formation rates are shown in FIGS. 13-24 for sDA-BE-TTER and FIGS. 1-12 for sDA-BE-TT. We also found that inserting an additional bipartite-NLS signal in between the UGIs of the sDA2.2 (SEQ ID NO:11) further improves editing rates, particularly at the low-editing PDCD1 gRNA site (FIG. 52).

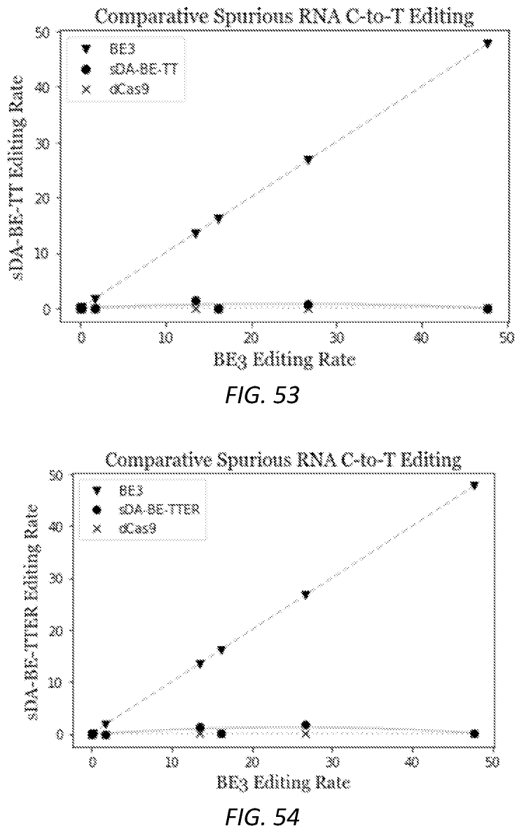

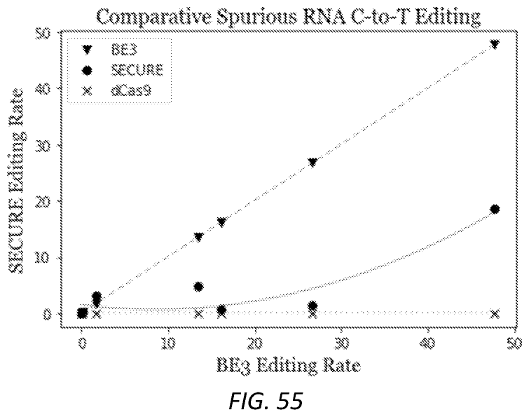

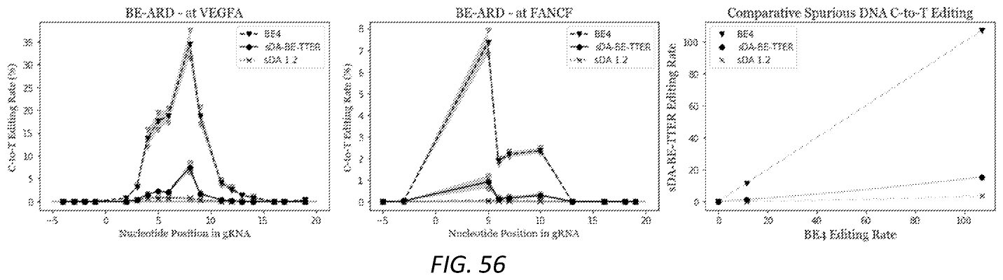

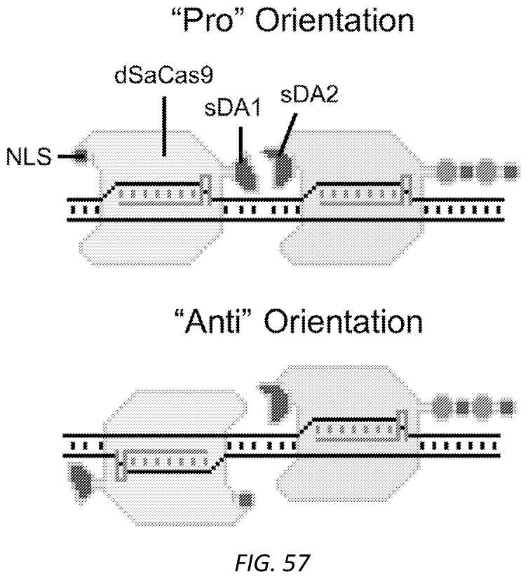

sDA-BE-TT and sDA-BE-TTER show dramatically low rates of spurious RNA editing compared to BE3 and R33A SECURE-BE3 (Grunewald et al., “Transcriptome-wide off-target RNA editing induced by CRISPR-guided DNA base editors,” Nature 569(7756):433-437 (2019)). Across 6 highly edited RNA sites that have been previously shown as BE3 substrates, RNA editing rates of sDA-BE-TT and sDA-BE-TTER are ˜30-50 fold lower than BE3 and ˜10 fold lower than the R33A SECURE BE3 editors (FIGS. 53-55). To determine the capacity of sDA-BE-TTER to create spurious DNA edits, we developed a simple assay by generating a stable in situ R-loop using a guided catalytically dead Cas9 (dCas9), which serves as the ssDNA substrate for an unguided BE with an orthogonal Cas9 domain co-expressed in the same cell. The editing activity of the BE on the R-loop can be quantified using targeted amplicon sequencing and used to determine the capacity of the BE for spurious off-target deamination events. Note that the dCas9 and BE must use orthogonal Cas9 (or Cas9-like) targeters so that the BE cannot be guided to the target site using the dCas9 gRNA. We call this the Base Editing at Anchored R-Loop DNA (BE-ARD) Assay. Using BE-ARD, we determined that the capacity of an sDA-BE-TTER editor with SaCas9 to conduct at an unguided ssDNA substrate is ˜10 fold lower than SaCas9 versions of both BE4 and BE4-Max. (FIG. 56).

SDA1.2-TTE
(SEQ ID NO: 8)
MKRTADGSEFESPKKKRKVSSETGPVAVDPTLRRRIEPHEFEVFFDPREL
RKETCLLYEINWGGRHSIWRHTSQNTNKHVEVNFIEKFTTE
sDA1.2-TTER
(SEQ ID NO: 9)
MKRTADGSEFESPKKKRKVSSETGPVAVDPTLRRRIEPHEFEVFFDPREL
RKETCLLYEINWGGRHSIWRHTSQNTNKHVEVNFIEKFTTER
sDA-BE-TT sDA1.2
(SEQ ID NO: 10)
MKRTADGSEFESPKKKRKVSSETGPVAVDPTLRRRIEPHEFEVFFDPREL
RKETCLLYEINWGGRHSIWRHTSQNTNKHVEVNFIEKFTT
sDA-BE-TT/-TTER sDA2.2 with an additional
bipartite-NLS signal in between the UGIs
(SEQ ID NO: 11)
MGNTRCRITWFLSWSPCGECSRAITEFLSRYPHVTLFIYIARLYHHADPR
NRQGLRDLISSGVTIQIMTEQESGYCWRNFVNYSPSNEAHWPRYPHLWVR
LYVLELYCIILGLPPCLNILRRKQPQLTFFTIALQSCHYQRLPPHILWAT
GLKSGGSSGGSSGSETPGTSESATPESSGGSSGGSDKKYSIGLAIGTNSV
GWAVITDEYKVPSKKFKVLGNTDRHSIKKNLIGALLFDSGETAEATRLKR
TARRRYTRRKNRICYLQEIFSNEMAKVDDSFFHRLEESFLVEEDKKHERH
PIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARL
SKSRRLENLIAQLPGEKKNGLFGNLIALSLGLTPNFKSNFDLAEDAKLQL
SKDTYDDDLDNLLAQIGDQYADLFLAAKNLSDAILLSDILRVNTEITKAP
LSASMIKRYDEHHQDLTLLKALVRQQLPEKYKEIFFDQSKNGYAGYIDGG
ASQEEFYKFIKPILEKMDGTEELLVKLNREDLLRKQRTFDNGSIPHQIHL
GELHAILRRQEDFYPFLKDNREKIEKILTFRIPYYVGPLARGNSRFAWMT
RKSEETITPWNFEEVVDKGASAQSFIERMTNFDKNLPNEKVLPKHSLLYE
YFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDIL
EDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGK
SDNVPSEEVVKKMKNYWRQLLNAKLITQRKFDNLTKAERGGLSELDKAGF
IKRQLVETRQITKHVAQILDSRMNTKYDENDKLIREVKVITLKSKLVSDF
RKDFQFYKVREINNYHHAHDAYLNAVVGTALIKKYPKLESEFVYGDYKVY
DVRKMIAKSEQEIGKATAKYFFYSNIMNFFKTEITLANGEIRKRPLIETN
GETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGI
TIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYL
DEIIEQISEFSKRVILADANLDKVLSAYNKHRDKPIREQAENIIHLFTLT
NLGAPAAFKYFDTTIDRKRYTSTKEVLDATLIHQSITGLYETRIDLSQLG
GDSGGSGGSGGSTNLSDIIEKETGKQLVIQESILMLPEEVEEVIGNKPES
DILVHTAYDESTDENVMLLTSDAPEYKPWALVIQDSNGENKIKMLSGGSG
KRTADGSEFEPKKKRKVGSGGSTNLSDIIEKETGKQLVIQESILMLPEEV
EEVIGNKPESDILVHTAYDESTDENVMLLTSDAPEYKPWALVIQDSNGEN
KIKMLSGGSKRTADGSEFEPKKKRKV

### Example 5: Variants of sDA-BE-TTER

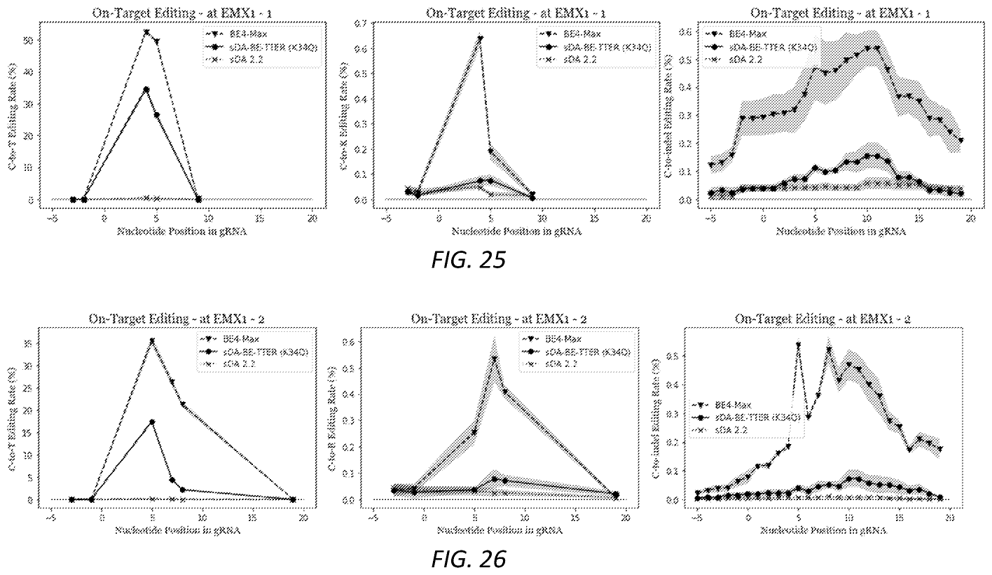

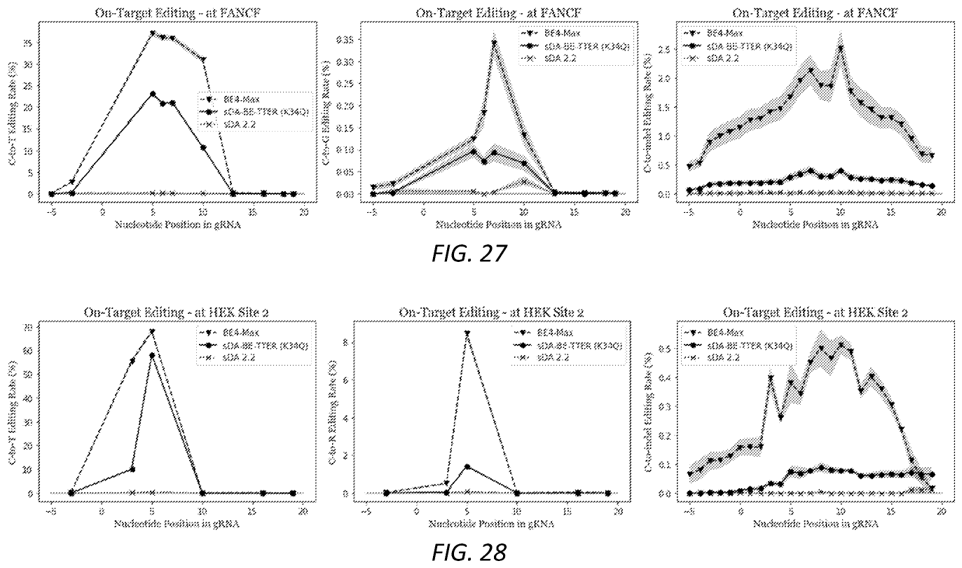

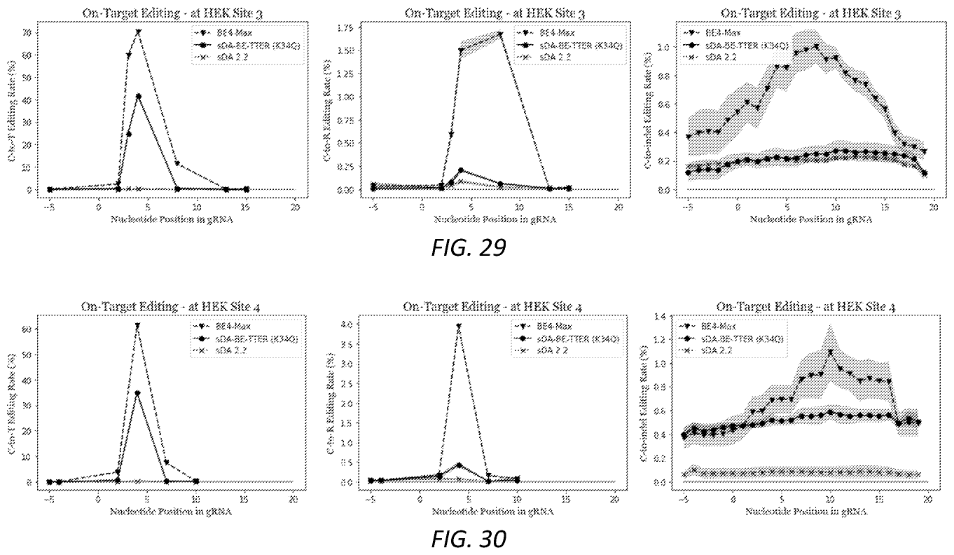

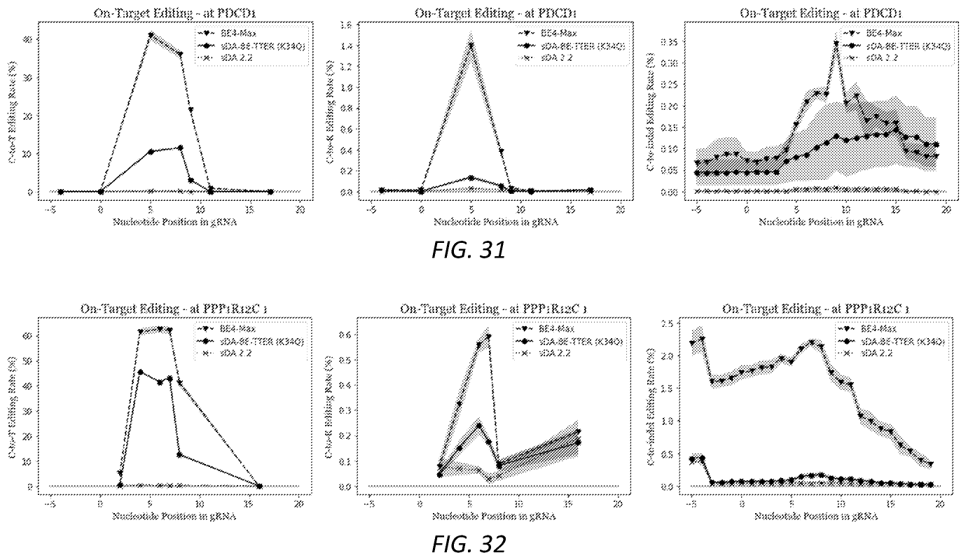

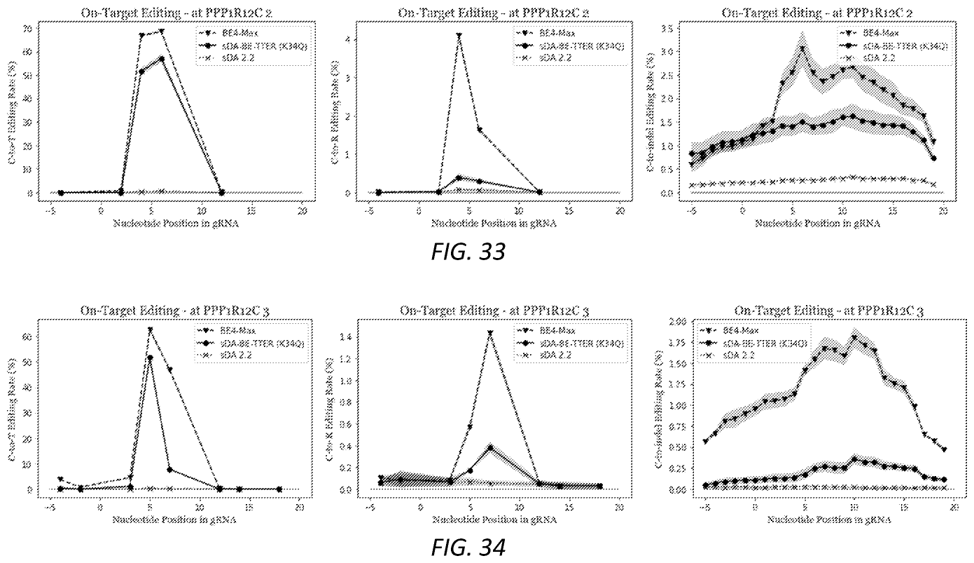

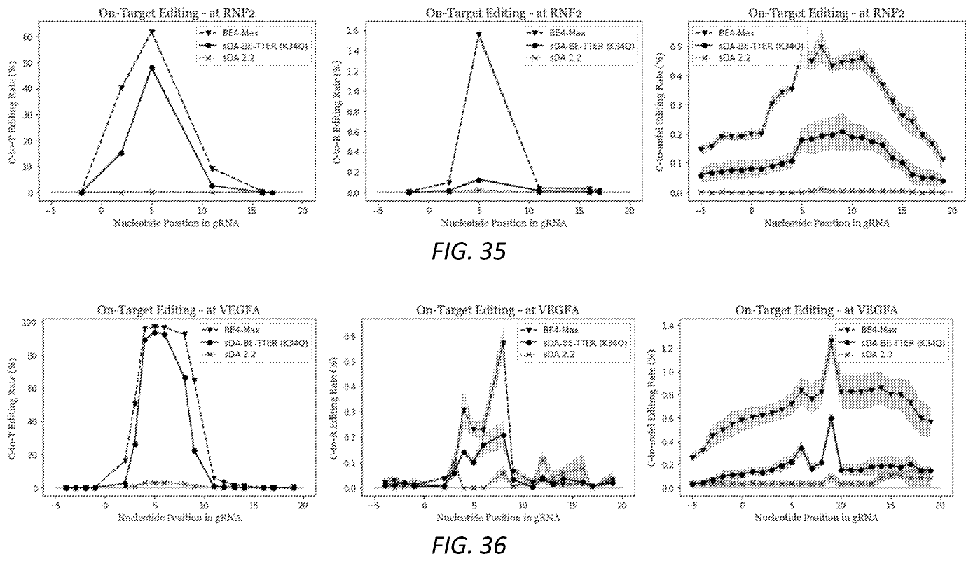

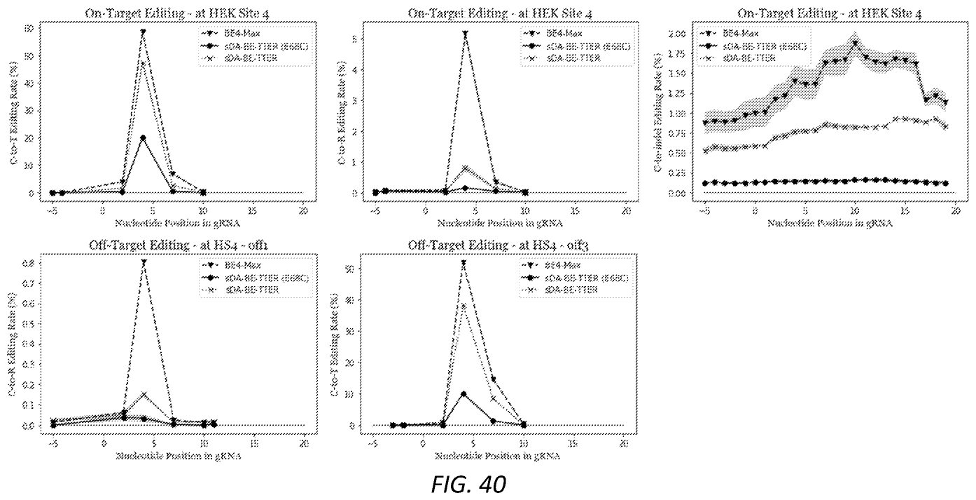

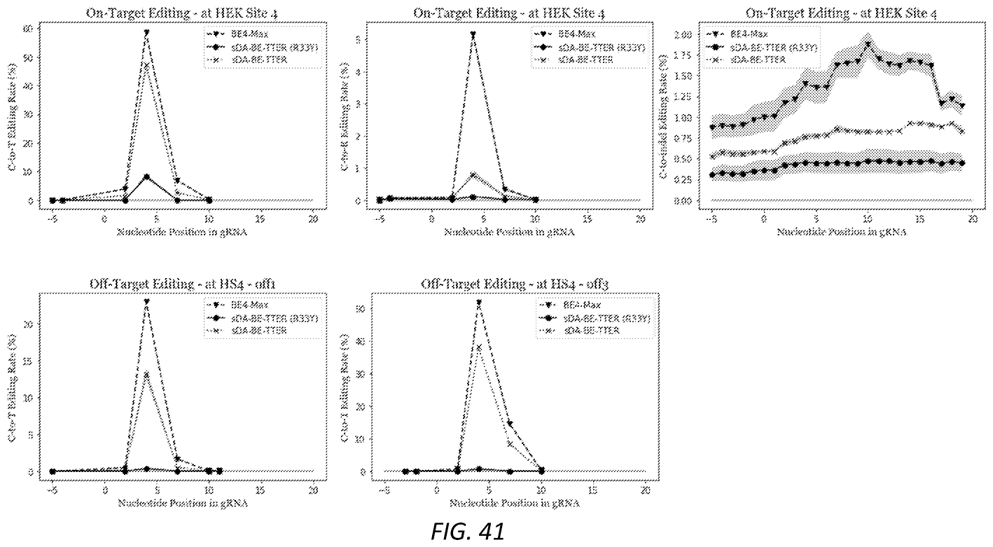

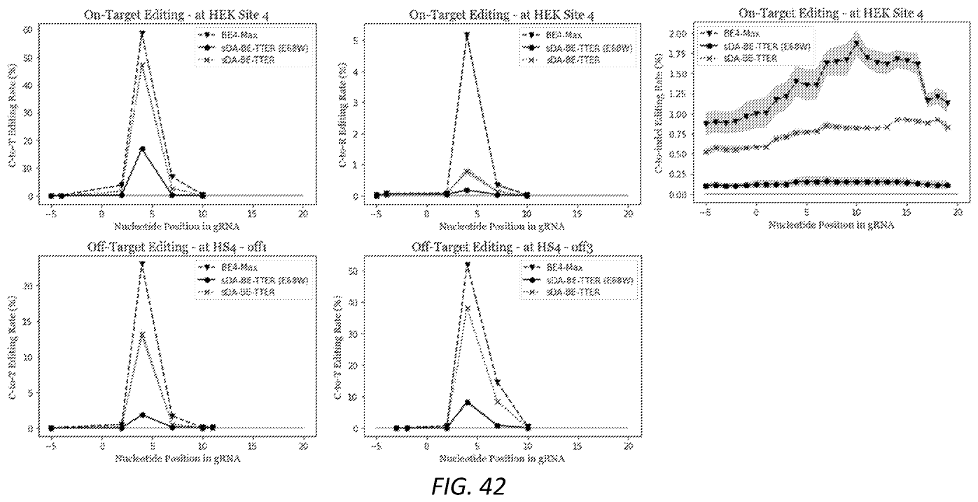

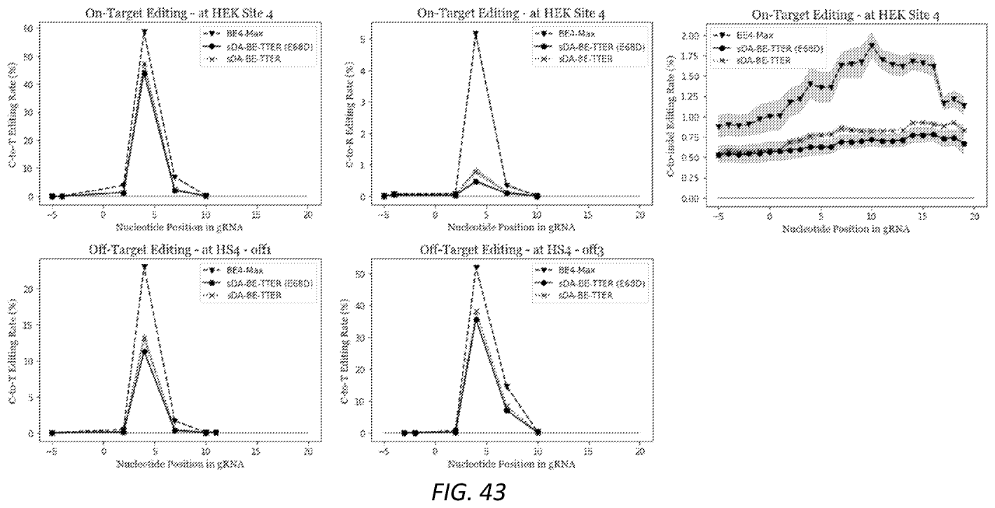

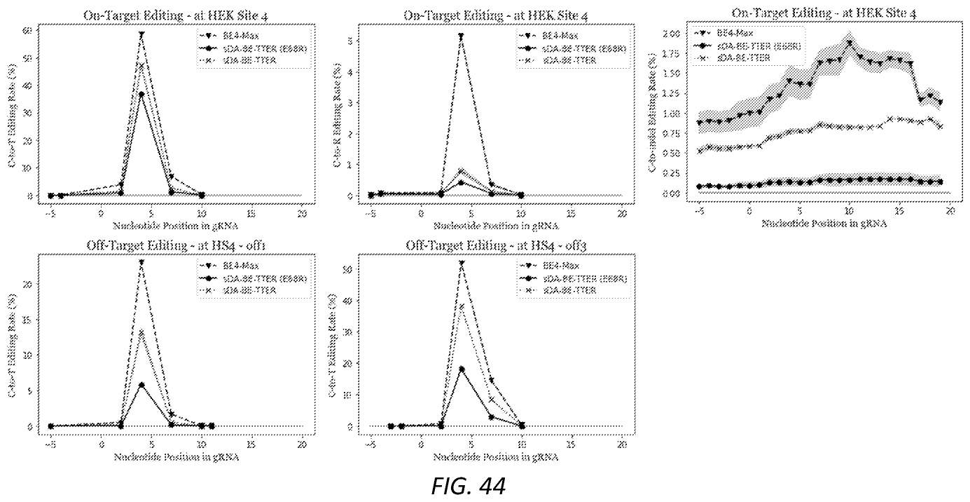

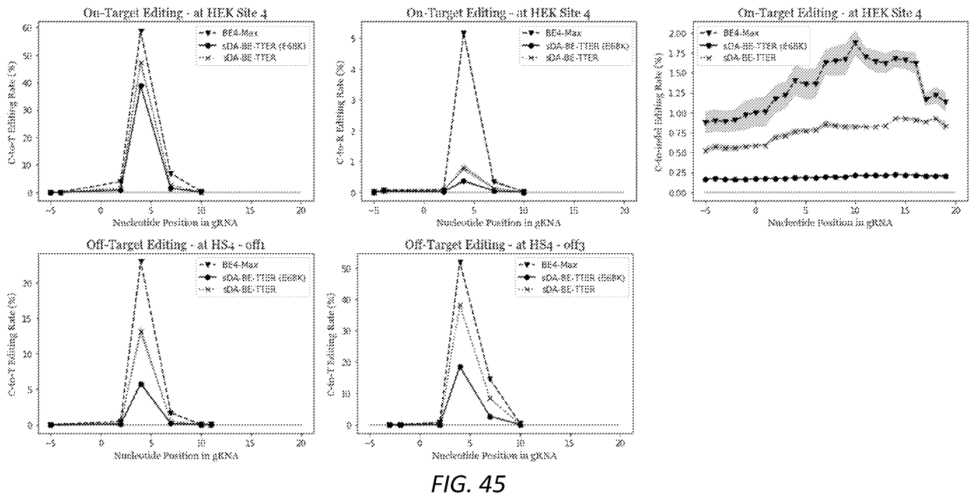

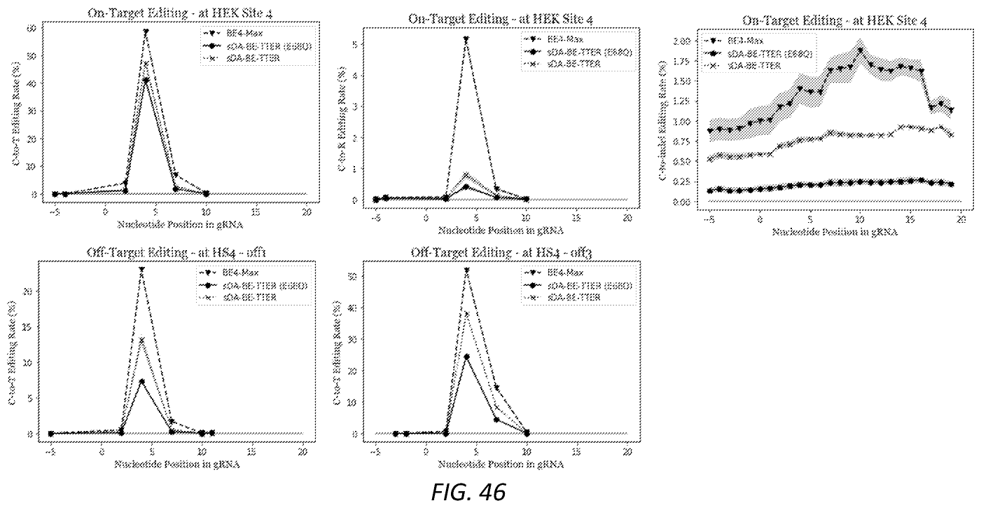

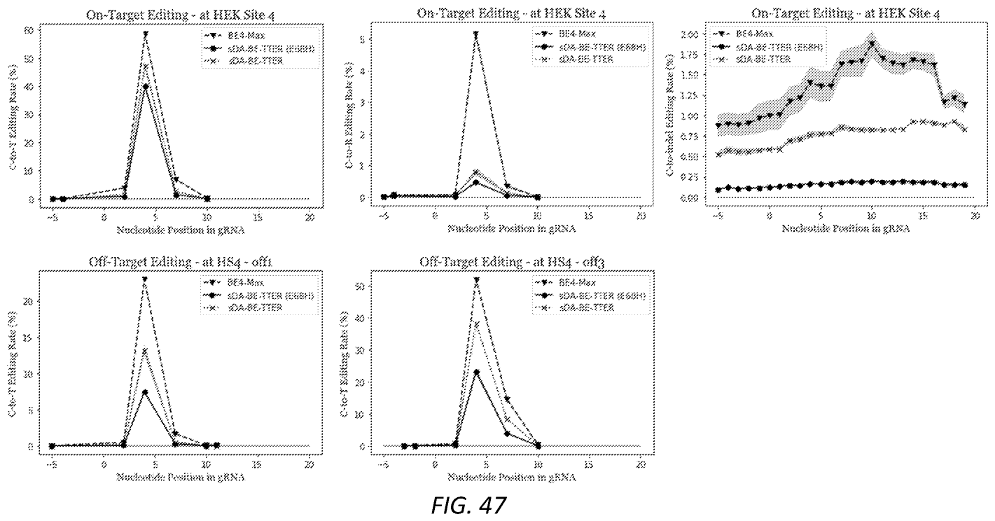

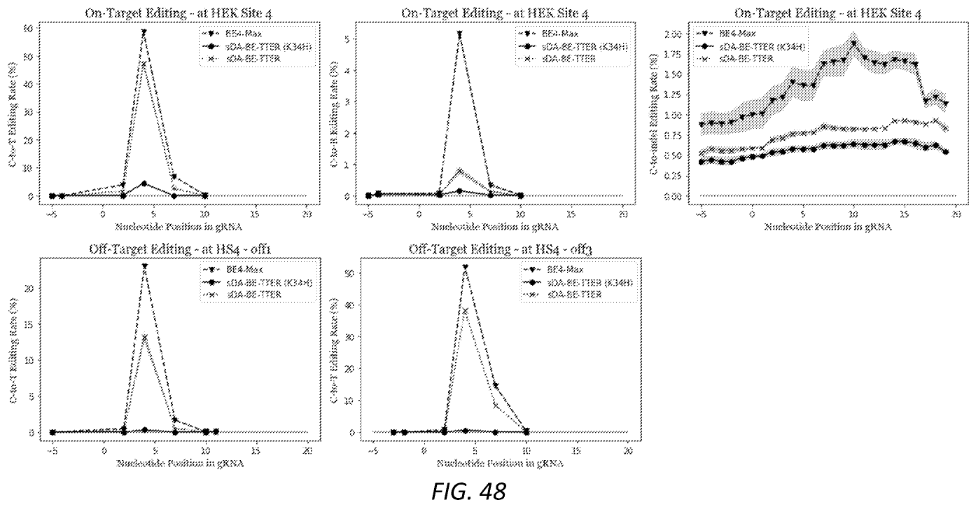

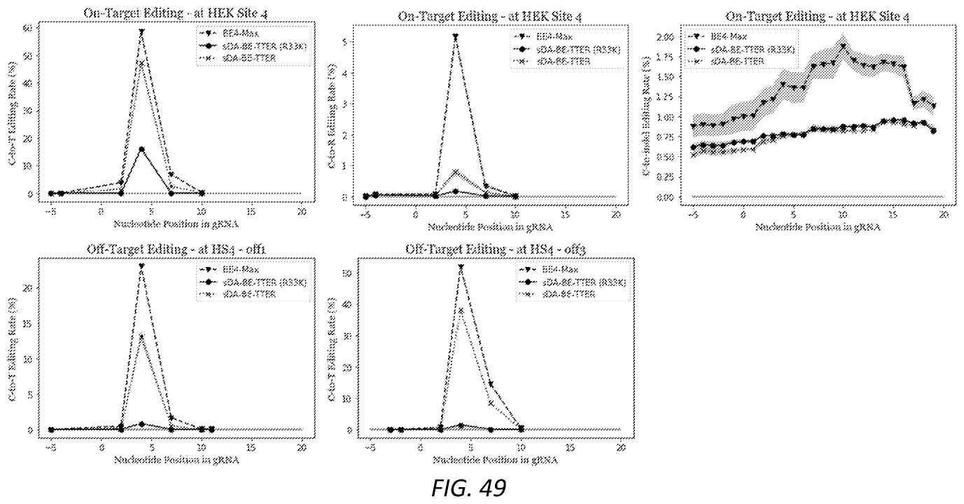

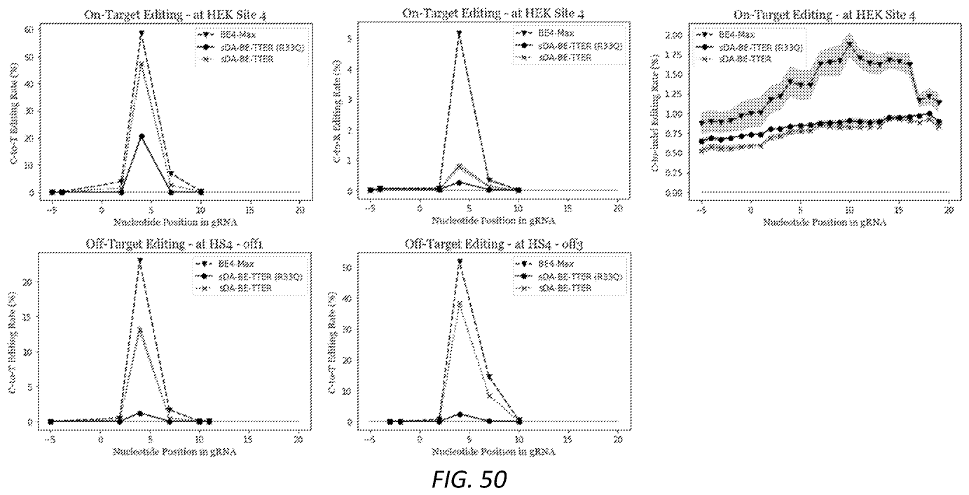

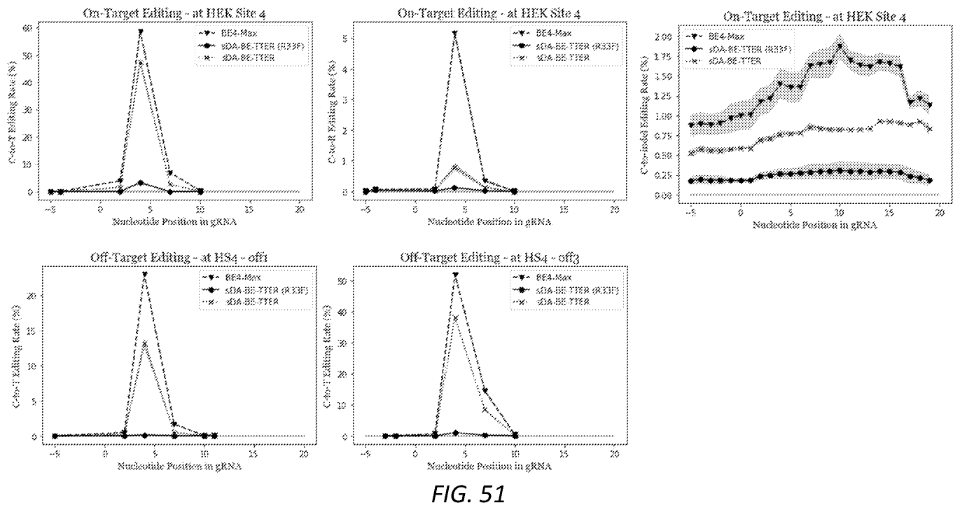

Furthermore, we show that variants of sDA-BE-TTER with mutations at residues corresponding to R33, K34 (sites previously identified as critical to spurious RNA editing—e.g. as SECURE mutations, corresponding to R51, K52 of BE4-Max (SEQ ID NO:1)) and E68 in the rAPOBEC1, corresponding to S101 of BE4-Max (SEQ ID NO:1) (SECURE-Like Orthologs [SLOs]—listed in Table 5 (SEQ ID NOs:12-26))) display substantial on-target activity while specificity than the parent editor (FIGS. 25-36 and 40-51). We hypothesize that mutations to these residues modulate the reaction rate (k_{cat}) of the deaminase enzyme and that slower enzymes will favor on-target editing at the expense of off-target edits due to the accumulation of the BE at the on-target site in the cell. For this reason, we also imagine that sDA-BEs and BEs in general bearing these mutations will also have lower rates of spurious DNA off-target editing and lower rates of spurious RNA off-target editing.

TABLE 5
Variants of sDA-BE-TTER sDA1.2 with SECURE-Like
Ortholog (SLO) Mutations

| SECURE-Like | SEQ | |
|---|---|---|
| Ortholog | ID | |
| Mutation | NO: | Sequence |
| R33G | 12 | MKRTADGSEFESPKKKRKVSSETGPVAVDPT |
| | | LRRRIEPHEFEVFFDPRELGKETCLLYEINW |
| | | GGRHSIWRHTSQNTNKHVEVNFIEKFTTER |
| R33H | 13 | MKRTADGSEFESPKKKRKVSSETGPVAVDPT |
| | | LRRRIEPHEFEVFFDPRELHKETCLLYEINW |
| | | GGRHSIWRHTSQNTNKHVEVNFIEKFTTER |
| R33Y | 14 | MKRTADGSEFESPKKKRKVSSETGPVAVDPT |
| | | LRRRIEPHEFEVFFDPRELYKETCLLYEINW |
| | | GGRHSIWRHTSQNTNKHVEVNFIEKFTTER |
| R33F | 15 | MKRTADGSEFESPKKKRKVSSETGPVAVDPT |
| | | LRRRIEPHEFEVFFDPRELFKETCLLYEINW |
| | | GGRHSIWRHTSQNTNKHVEVNFIEKFTTER |
| R33A | 16 | MKRTADGSEFESPKKKRKVSSETGPVAVDPT |
| | | LRRRIEPHEFEVFFDPRELAKETCLLYEINW |
| | | GGRHSIWRHTSQNTNKHVEVNFIEKFTTER |
| K34Q | 17 | MKRTADGSEFESPKKKRKVSSETGPVAVDPT |
| | | LRRRIEPHEFEVFFDPRELRQETCLLYEINW |
| | | GGRHSIWRHTSQNTNKHVEVNFIEKFTTER |
| K34H | 18 | MKRTADGSEFESPKKKRKVSSETGPVAVDPT |
| | | LRRRIEPHEFEVFFDPRELRHETCLLYEINW |
| | | GGRHSIWRHTSQNTNKHVEVNFIEKFTTER |
| K34N | 19 | MKRTADGSEFESPKKKRKVSSETGPVAVDPT |
| | | LRRRIEPHEFEVFFDPRELRNETCLLYEINW |
| | | GGRHSIWRHTSQNTNKHVEVNFIEKFTTER |
| E68C | 20 | MKRTADGSEFESPKKKRKVSSETGPVAVDPT |
| | | LRRRIEPHEFEVFFDPRELRKETCLLYEINW |
| | | GGRHSIWRHTSQNTNKHVEVNFICKFTTER |
| E68K | 21 | MKRTADGSEFESPKKKRKVSSETGPVAVDPT |
| | | LRRRIEPHEFEVFFDPRELRKETCLLYEINW |
| | | GGRHSIWRHTSQNTNKHVEVNFIKKFTTER |
| E68Q | 22 | MKRTADGSEFESPKKKRKVSSETGPVAVDPT |
| | | LRRRIEPHEFEVFFDPRELRKETCLLYEINW |
| | | GGRHSIWRHTSQNTNKHVEVNFIQKFTTER |
| E68W | 23 | MKRTADGSEFESPKKKRKVSSETGPVAVDPT |
| | | LRRRIEPHEFEVFFDPRELRKETCLLYEINW |
| | | GGRHSIWRHTSQNTNKHVEVNFIWKFTTER |
| E68H | 24 | MKRTADGSEFESPKKKRKVSSETGPVAVDPT |
| | | LRRRIEPHEFEVFFDPRELRKETCLLYEINW |
| | | GGRHSIWRHTSQNTNKHVEVNFIKKFTTER |
| E68D | 25 | MKRTADGSEFESPKKKRKVSSETGPVAVDPT |
| | | LRRRIEPHEFEVFFDPRELRKETCLLYEINW |
| | | GGRHSIWRHTSQNTNKHVEVNFIDKFTTER |
| E68R | 26 | MKRTADGSEFESPKKKRKVSSETGPVAVDPT |
| | | LRRRIEPHEFEVFFDPRELRKETCLLYEINW |
| | | GGRHSIWRHTSQNTNKHVEVNFIRKFTTER |

TABLE 6
Sequences of various additional embodiments of
sDAs are set forth in the table below.

| | sDA 1.2 component | sDA 2.2 component |
|---|---|---|
| | SEQ ID NO: | SEQ ID NO: |
| SDA-BE4.1 | 2 | 74 |
| SDA-BE4.2 | 9 | 74 |
| SDA-BE4.3 | 2 | 5 |
| SDA-BE4.4 | 9 | 5 |
| sD A-BE4.3-Max | 2 | 11 |
| sD A-BE4.4-Max | 9 | 11 |

sDA-BE sDA 2.2 (GNTR variant)(SEQ ID NO: 74)
MGNTRCSITWFLSWSPCGECSRAITEFLSRYPHVTLFIYIARLYHHADPR
NRQGLRDLISSGVTIQIMTEQESGYCWRNFVNYSPSNEAHWPRYPHLWVR
LYVLELYCIILGLPPCLNILRRKQPQLTFFTIALQSCHYQRLPPHILWAT
GLKSGGSSGGSSGSETPGTSESATPESSGGSSGGSDKKYSIGLAIGTNSV
GWAVITDEYKVPSKKFKVLGNTDRHSIKKNLIGALLFDSGETAEATRLKR
TARRRYTRRKNRICYLQEIFSNEMAKVDDSFFHRLEESFLVEEDKKHERH
PIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARL
SKSRRLENLIAQLPGEKKNGLFGNLIALSLGLTPNFKSNFDLAEDAKLQL
SKDTYDDDLDNLLAQIGDQYADLFLAAKNLSDAILLSDILRVNTEITKAP
LSASMIKRYDEHHQDLTLLKALVRQQLPEKYKEIFFDQSKNGYAGYIDGG
ASQEEFYKFIKPILEKMDGTEELLVKLNREDLLRKQRTFDNGSIPHQIHL
GELHAILRRQEDFYPFLKDNREKIEKILTFRIPYYVGPLARGNSRFAWMT
RKSEETITPWNFEEVVDKGASAQSFIERMTNFDKNLPNEKVLPKHSLLYE
YFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDIL
EDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGK
SDNVPSEEVVKKMKNYWRQLLNAKLITQRKFDNLTKAERGGLSELDKAGF
IKRQLVETRQITKHVAQILDSRMNTKYDENDKLIREVKVITLKSKLVSDF
RKDFQFYKVREINNYHHAHDAYLNAVVGTALIKKYPKLESEFVYGDYKVY
DVRKMIAKSEQEIGKATAKYFFYSNIMNFFKTEITLANGEIRKRPLIETN
GETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGI
TIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYL
DEIIEQISEFSKRVILADANLDKVLSAYNKHRDKPIREQAENIIHLFTLT
NLGAPAAFKYFDTTIDRKRYTSTKEVLDATLIHQSITGLYETRIDLSQLG
GDSGGSGGSGGSTNLSDIIEKETGKQLVIQESILMLPEEVEEVIGNKPES
DILVHTAYDESTDENVMLLTSDAPEYKPWALVIQDSNGENKIKMLSGGSG
GSGGSTNLSDIIEKETGKQLVIQESILMLPEEVEEVIGNKPESDILVHTA
YDESTDENVMLLTSDAPEYKPWALVIQDSNGENKIKMLSGGSKRTADGSE
FEPKKKRKV

### Example 6: Dimeric and Gate sDA-BE

We also sought to create a dimeric AND Gate sDA-BE architecture that makes use of adjacently-targeting Cas9 molecules. In this strategy, the C-terminal sDA piece remains attached to the nCas9-UGI-UGI domain of BE4Max, while the sDA-1 piece is fused to the C-terminus of an orthogonal catalytically inactivated Staphylococcus aureus Cas9 (dSaCas9) domain. To ensure maximum targeting range, the previously-described engineered KKH variant of SaCas9 was used, which recognizes a relaxed PAM sequence of NNNRRT (SEQ ID NO:27) (Kleinstiver et al., “Broadening the targeting range of Staphylococcus aureus CRISPR-Cas9 by modifying PAM recognition,” Nat Biotechnol 33, 1293-1298 (2015)). While one might expect that such a configuration would support on-target editing from solution as in the molecularity model, a successful co-targeting of both domains to the same site in a productive geometric orientation should improve the on-target editing while leaving spurious editing unchanged from a model in which the spurious DNA editing diminishment by sDA-BEs is explained by a difference in molecularity between on-target and spurious DNA target sites, thus leading to an exaggerated on-target:spurious editing ratio and allowing sDA-BEs to achieve higher on-target editing rates at even lower concentrations of nuclear deaminase. We created and tested two versions of this dimeric enzyme—one that mimics the sDA-BE4.2 architecture (comprising proteins encoded by dSaCas9 (KKH) sDA1 (SEQ ID NO:29) and an sDA2.2-BE lacking the S83R mutation (SEQ ID NO:74) and any associated gRNA targeting molecules) and one that uses the sDA-BE4.4-Max architecture (comprising proteins encoded by dSaCas9 (KKH) sDA1 (SEQ ID NO:29) and sDA-BE-TT/-TTER sDA2.2 with an additional bipartite-NLS signal in between the UGIs (SEQ ID NO:11) and any associated gRNA targeting molecules). That is, these dimeric sDA-BE enzymes match those corresponding architectures, but with an added dSaCas9 tethered to the C-terminal sDA piece.

dSaCas9 (KKH) sDA1
(SEQ ID NO: 29)
MGKRNYILGLAIGITSVGYGIIDYETRDVIDAGVRLFKEANVENNEGRRS
KRGARRLKRRRRHRIQRVKKLLFDYNLLTDHSELSGINPYEARVKGLSQK
LSEEEFSAALLHLAKRRGVHNVNEVEEDTGNELSTKEQISRNSKALEEKY
VAELQLERLKKDGEVRGSINRFKTSDYVKEAKQLLKVQKAYHQLDQSFID
TYIDLLETRRTYYEGPGEGSPFGWKDIKEWYEMLMGHCTYFPEELRSVKY
AYNADLYNALNDLNNLVITRDENEKLEYYEKFQIIENVFKQKKKPTLKQI
AKEILVNEEDIKGYRVTSTGKPEFTNLKVYHDIKDITARKEIIENAELLD
QIAKILTIYQSSEDIQEELTNLNSELTQEEIEQISNLKGYTGTHNLSLKA
INLILDELWHTNDNQIAIFNRLKLVPKKVDLSQQKEIPTTLVDDFILSPV
VKRSFIQSIKVINAIIKKYGLPNDIIIELAREKNSKDAQKMINEMQKRNR
QTNERIEEIIRTTGKENAKYLIEKIKLHDMQEGKCLYSLEAIPLEDLLNN
PFNYEVDHIIPRSVSFDNSFNNKVLVKQEEASKKGNRTPFQYLSSSDSKI
SYETFKKHILNLAKGKGRISKTKKEYLLEERDINRFSVQKDFINRNLVDT
RYATRGLMNLLRSYFRVNNLDVKVKSINGGFTSFLRRKWKFKKERNKGYK
HHAEDALIIANADFIFKEWKKLDKAKKVMENQMFEEKQAESMPEIETEQE
YKEIFITPHQIKHIKDFKDYKYSHRVDKKPNRKLINDTLYSTRKDDKGNT
LIVNNLNGLYDKDNDKLKKLINKSPEKLLMYHHDPQTYQKLKLIMEQYGD
EKNPLYKYYEETGNYLTKYSKKDNGPVIKKIKYYGNKLNAHLDITDDYPN
SRNKVVKLSLKPYRFDVYLDNGVYKFVTVKNLDVIKKENYYEVNSKCYEE
AKKLKKISNQAEFIASFYKNDLIKINGELYRVIGVNNDLLNRIEVNMIDI
TYREYLENMNDKRPPHIIKTIASKTQSIKKYSTDILGNLYEVKSKKHPQI
IKKGGSGGSKRTADGSEFESPKKKRKVSSETGPVAVDPTLRRRIEPHEFE
VFFDPRELRKETCLLYEINWGGRHSIWRHTSQNTNKHVEVNFIEKFTTER

We evaluated these dual-targeted sDA-BE architectures at 5 SpCas9 gRNA target sites (PP1R12C 1, PDCD1, RNF2, VEGFA, and HEK Site 3, Table 7) with a panel of co-expressed SaCas9 gRNAs targeting adjacent sites with varying characteristics. We categorized the SaCas9 gRNAs based on their directionality compared to the SpCas9 target site, with “Pro” sites facing the same direction as the SpCas9 target site and “Anti” facing the opposite direction. We also noted the PAM-to-PAM distance in terms of basepairs, with a positive number indicating a position downstream of the SpCas9 target site and a negative number indicating an upstream target site. Finally, we also varied the SaCas9 gRNA protospacer target length, ranging from 17 to 22 bps.

TABLE 7
SaCas9 Target Protospacer Sequences

| SpCas9 gRNA | SaCas9 gRNA Target | |
|---|---|---|
| target site | Sequence | SEQ ID NO: |
| PPP1R12C 1 | GGGATTGGAATGCCGGGGCGGG | 30 |
| PPP1R12C 1 | GGAATGCCGGGGCGGGGTGG | 31 |
| PPP1R12C 1 | GTAGGATTGCTGGAACCCTGCC | 32 |
| PPP1R12C 1 | GAAAGACCTGCGGCAGGGTTCC | 33 |
| PPP1R12C 1 | GCCGCAGGTCTTTCTGGGA | 34 |
| PPP1R12C 1 | GAGGGGATGCGTTTACTTGGGG | 35 |
| PPP1R12C 1 | GCCCCGGCATTCCAATCCC | 36 |
| PPP1R12C 1 | CCCACCCACCCCGCCCCGGCAT | 37 |
| PPP1R12C 1 | CCACCCCGCCCCGGCAT | 38 |
| PDCD1 | CATTCCGGAATGCCGGGGCGGGGTGG | 39 |
| PDCD1 | GTCCTGGCCGGGCTGGC | 40 |
| PDCD1 | GTTGTGTGACACGGAAGC | 41 |
| RNF2 | GCAGTTGTGTGACACGGAAGC | 75 |
| RNF2 | CATGTTCTAAAAATGTAT | 76 |
| RNF2 | GCATATGAGACGTGTAAAC | 77 |
| VEGFA | GGGGCATATGAGACGTGTAAAC | 78 |
| HEK Site 3 | GCCCAGAAGTTGGACGAAAAGT | 79 |

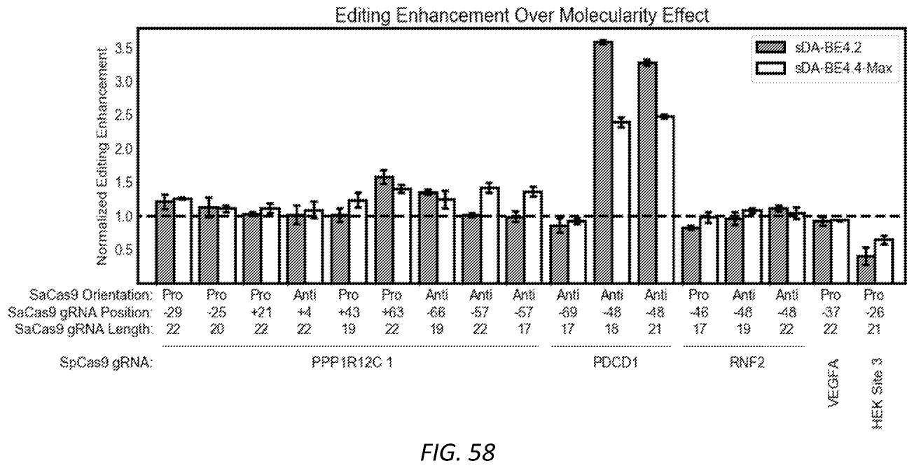

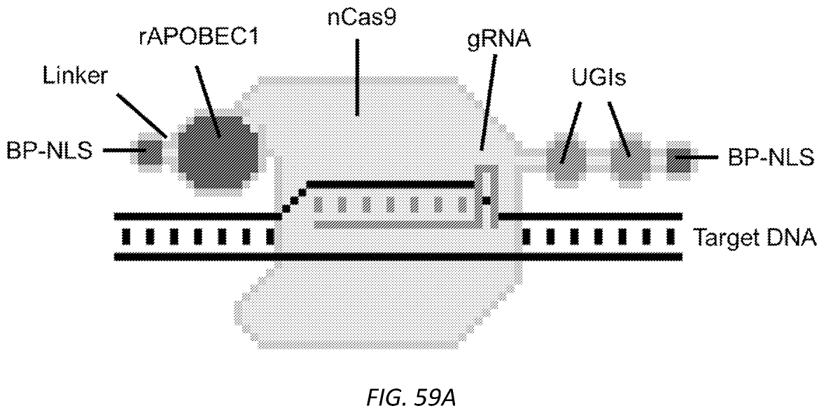

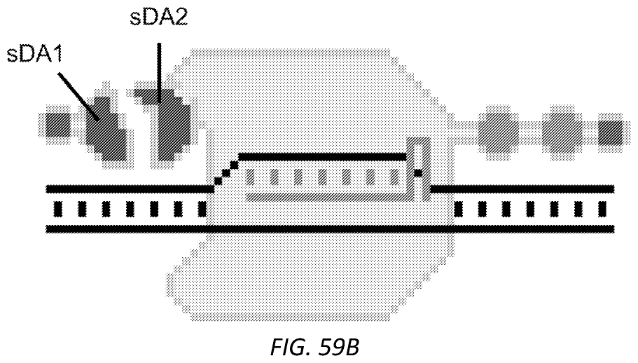

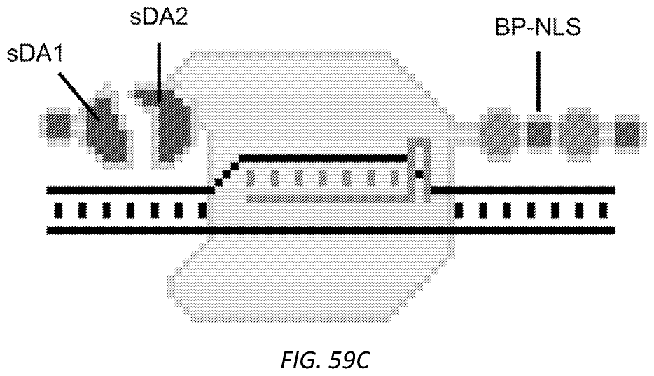

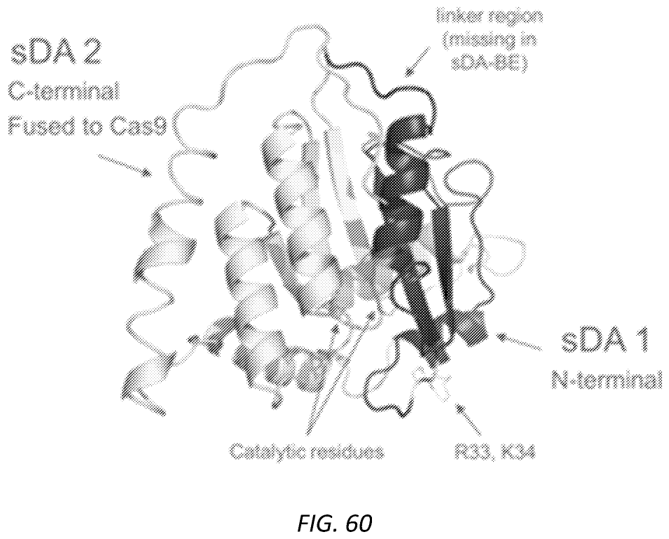

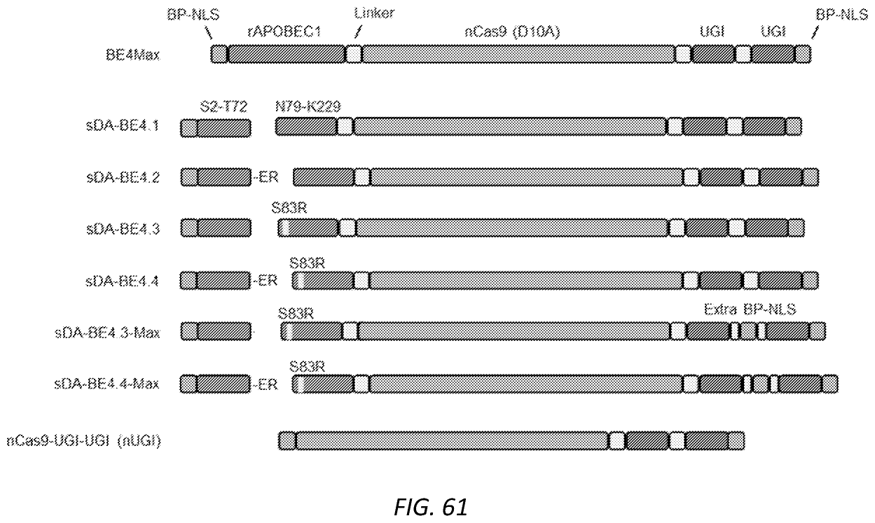

By running parallel experiments that evaluated the on-target editing rates and spurious DNA targeting rates using an SaCas9 BE-ARD assay in which the SaCas9 gRNA targeting the on-target site is replaced by a BE-ARD site, we were able to determine the ratio of on-target:spurious off target DNA editing ratio for every SpCas9/SaCas9 targeting pair. In a similar experiment, we then determined the analogous ratio under conditions where the same dimeric dual-targeting architecture lacked any SaCas9 gRNA such that any on-target editing was due to simple-reformation of the sDA pieces from solution, a scenario that mimics the prior sDA-BE approaches and whose diminishment of spurious DNA editing is likely based on a molecularity effect. By taking a ratio-of-ratios for matched SpCas9 gRNA target sites between these conditions, we can then determine if a dual-targeted sDA-BE architecture reduces spurious DNA editing with respect to spurious DNA editing under the molecularity effect as normalized by on-target editing. We call this metric a Normalized Editing Enhancement, and note that a value greater than 1.0 for this metric denotes an improvement in on-target editing over spurious DNA editing compared to an approach lacking the SaCas9 targeting domain. This data is summarized in FIG. 58. The SaCas9 gRNA protospacer target sequences are shown in Table 7 and appear in that table in the order they appear in FIG. 58. These SaCas9 protospacer sequences are fused to the 5′ end of the chimeric tracrRNA/crRNA sequence in SEQ ID NO: 80 to form a SaCas9 single gRNA construct, which are then expressed from an expression plasmid using the U6 promoter.

SaCas9 single gRNA sequence (lacking protospacer)
(SEQ ID NO: 80)
GUUUUAGUACUCUGUAAUGAAAAUUACAGAAUCUACUAAAACAAGGCA
AAAUGCCGUGUUUAUCUCGUCAACUUGUUGGCGAGA

This analysis revealed that a dual-targeting SpCas9/SaCas9 sDA-BE architecture can substantially enhance the on-target:spurious DNA off-target editing ratio at some sites, with two SaCas9 gRNA configurations resulting in two-to-three-fold enhancement of on-target activity, with several others also leading to a moderate enhancement. We theorize that further exploration of this approach will help to elucidate the rules governing dual-targeting sDA-BE architectures, and will therefore lead to an improved rate of on-target editing while achieving even lower rates of spurious off-target DNA editing than achievable but available CBE technology.

### Example 7: CGBE

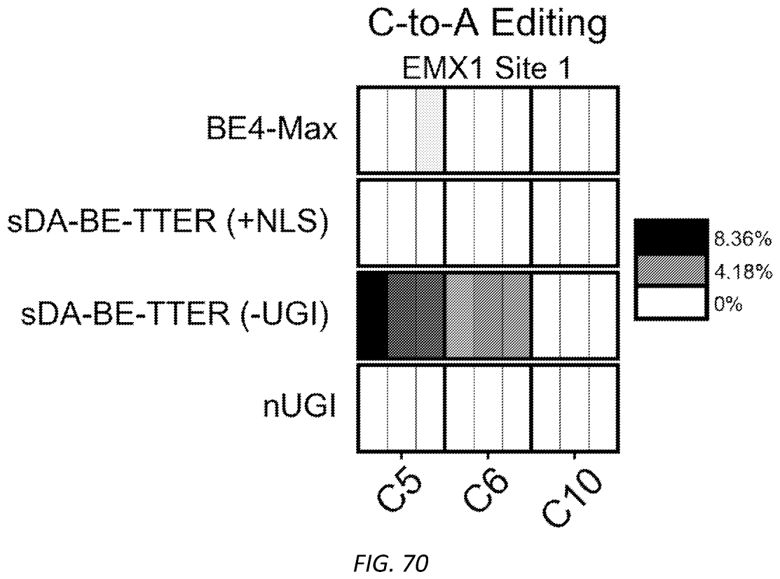

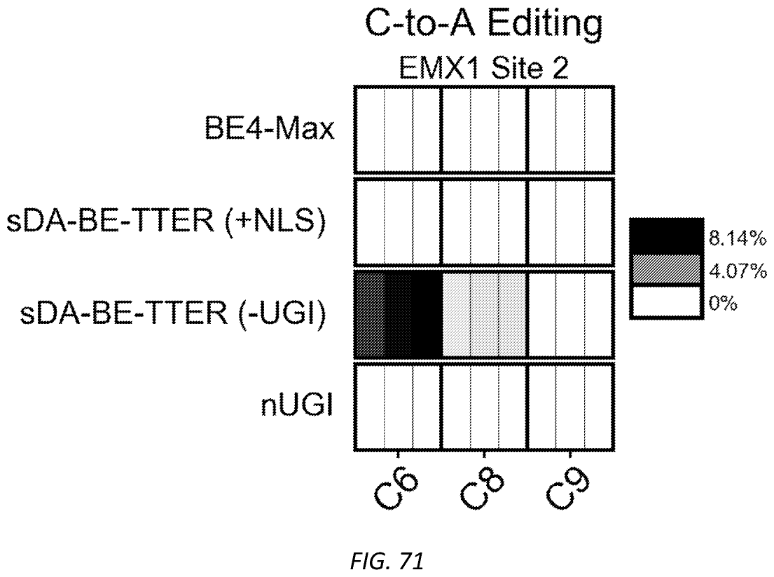

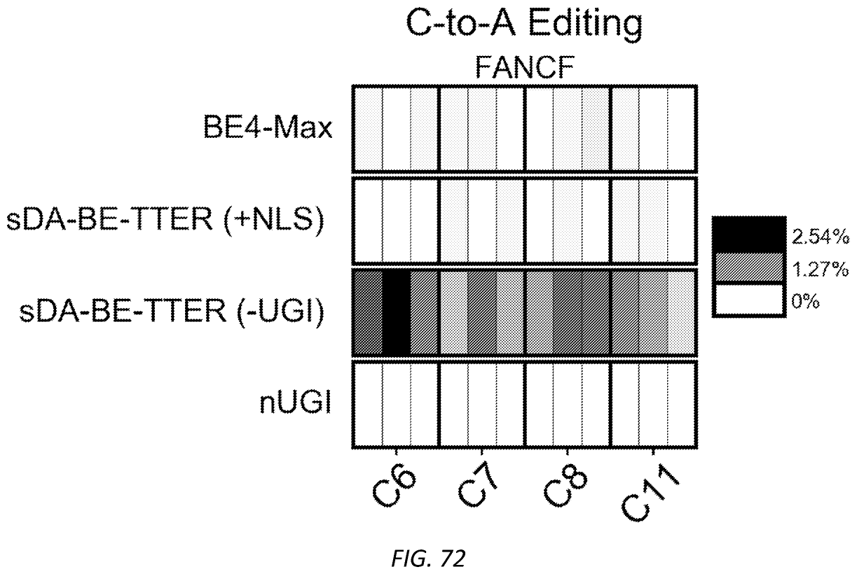

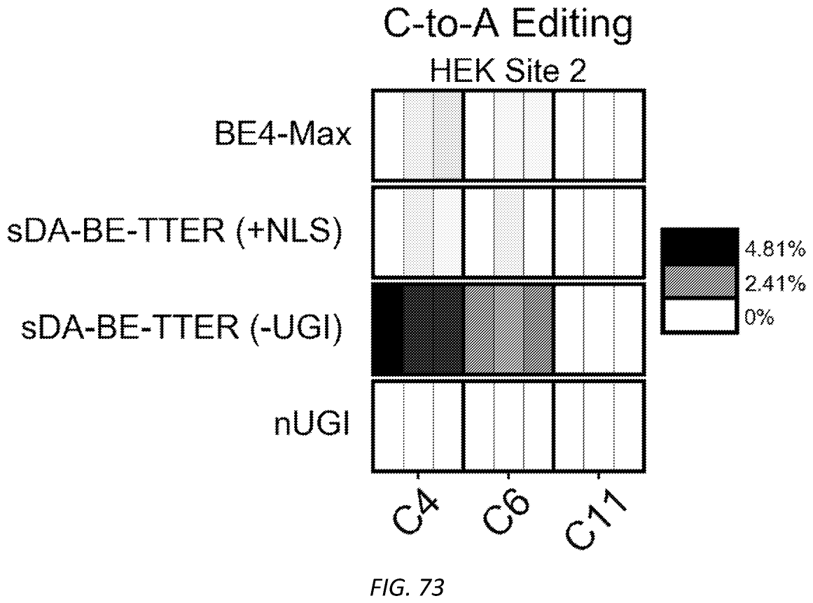

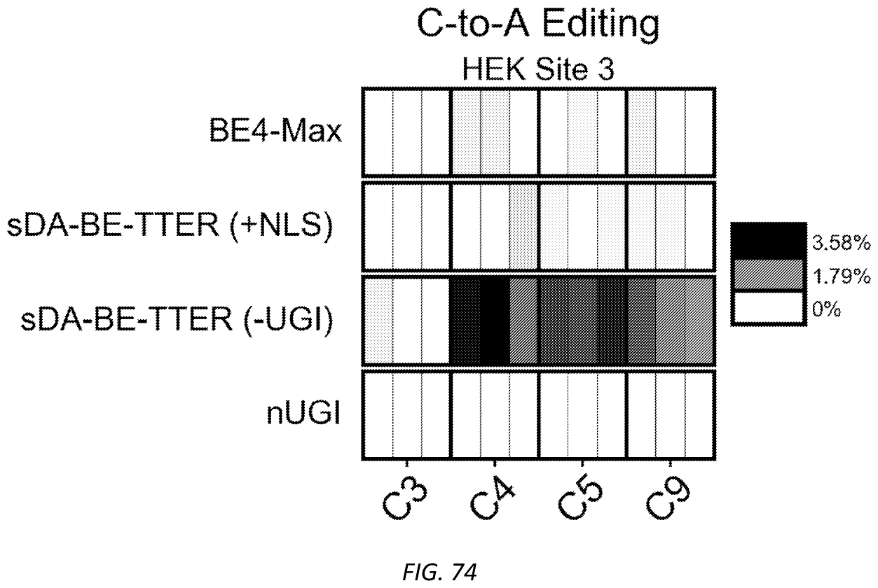

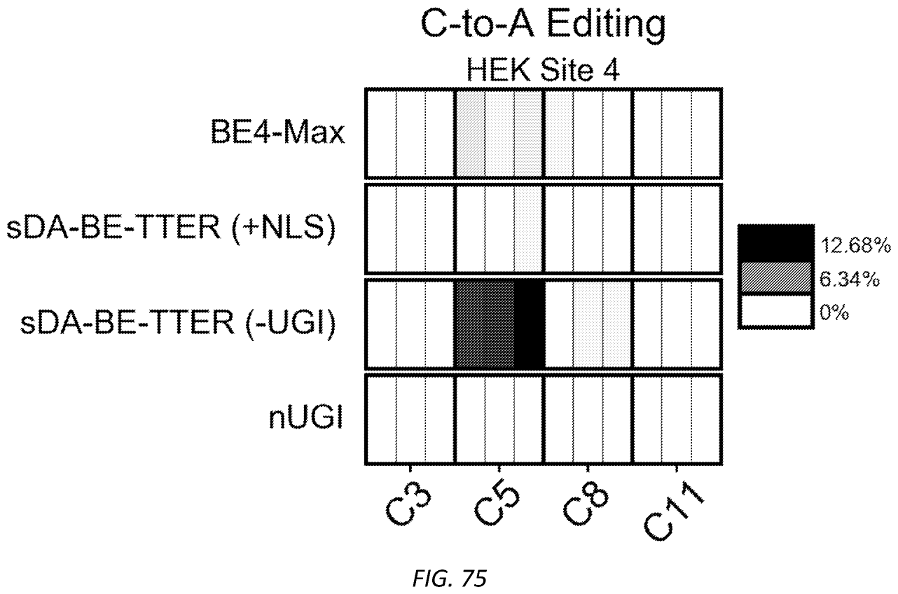

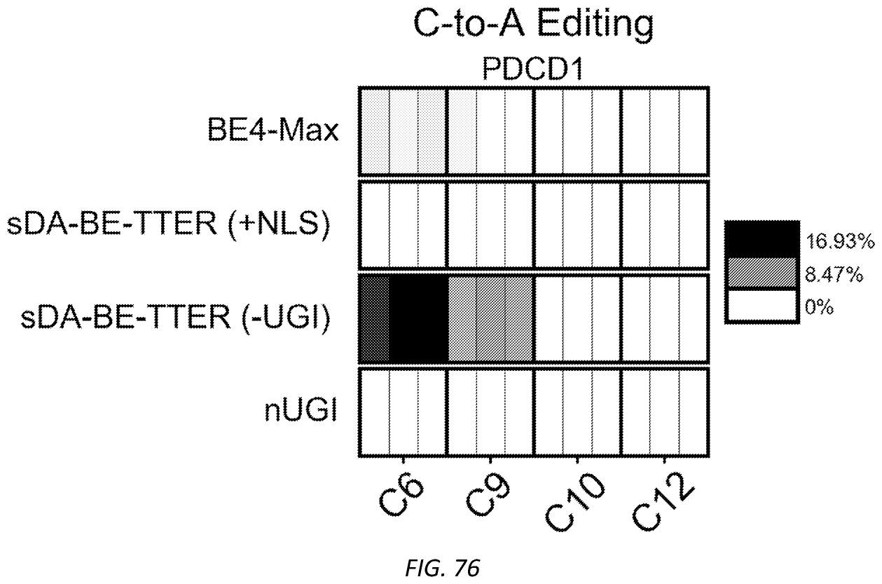

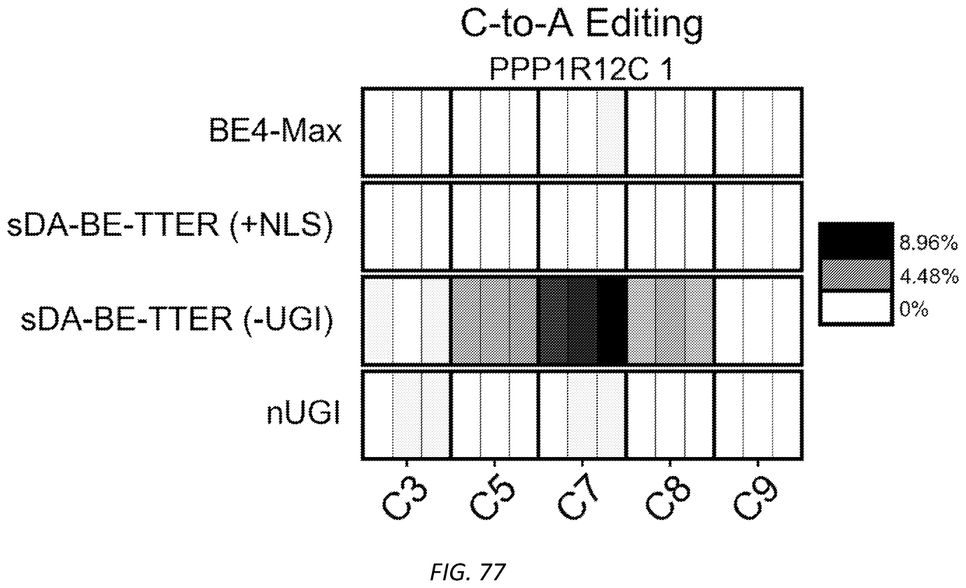

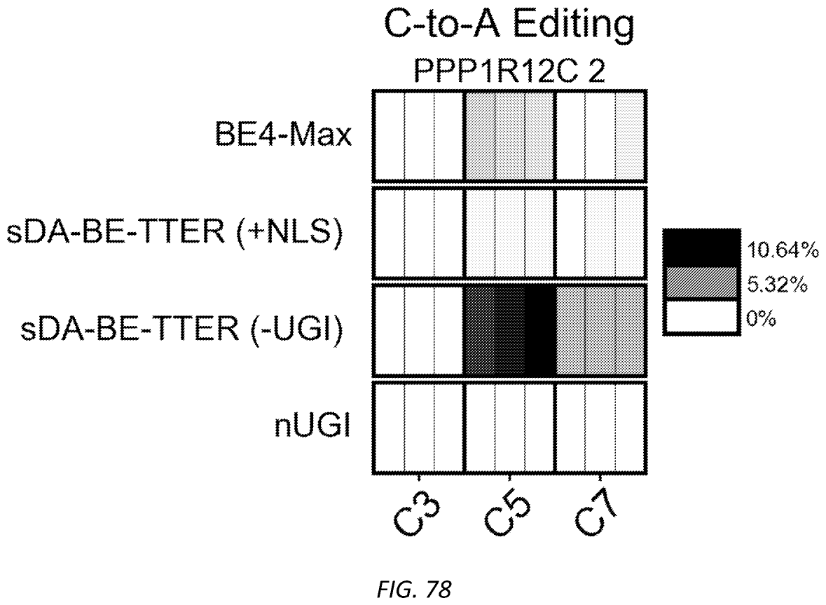

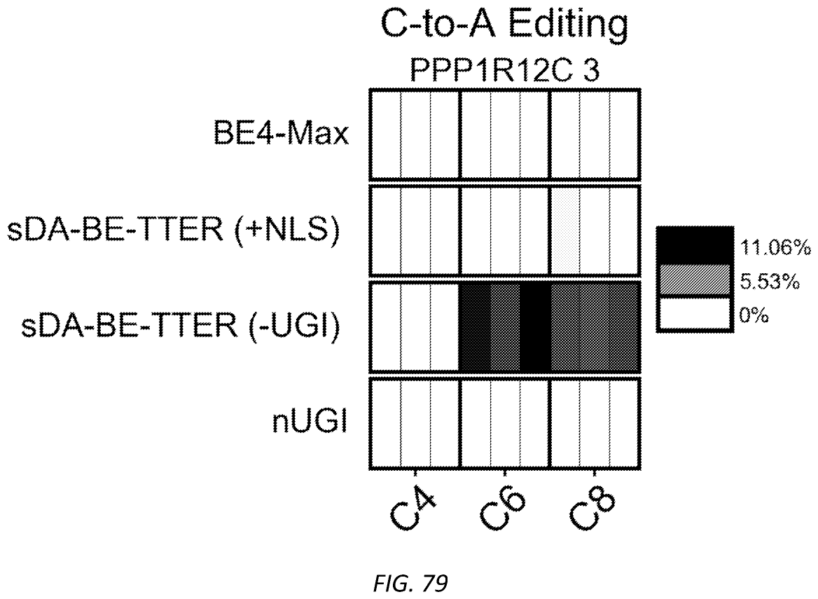

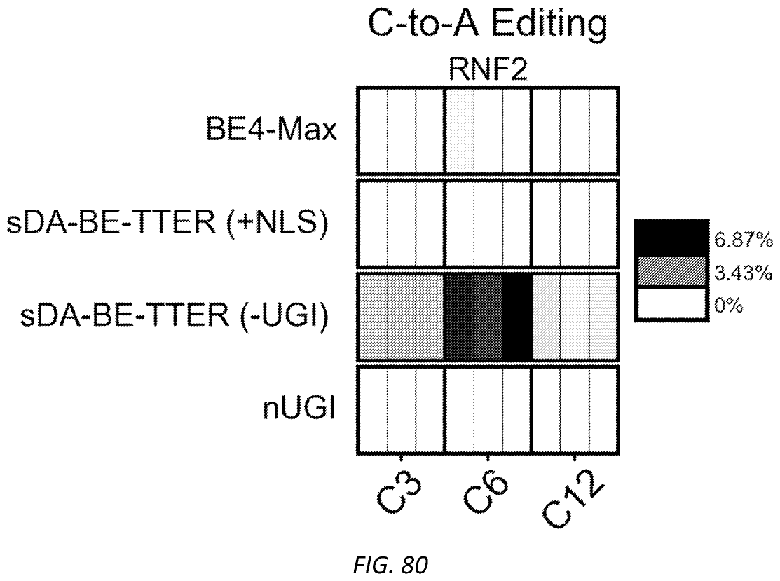

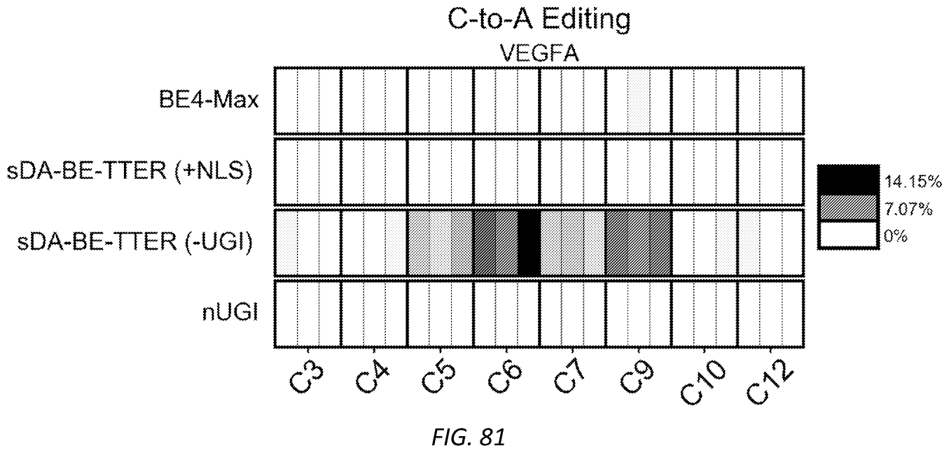

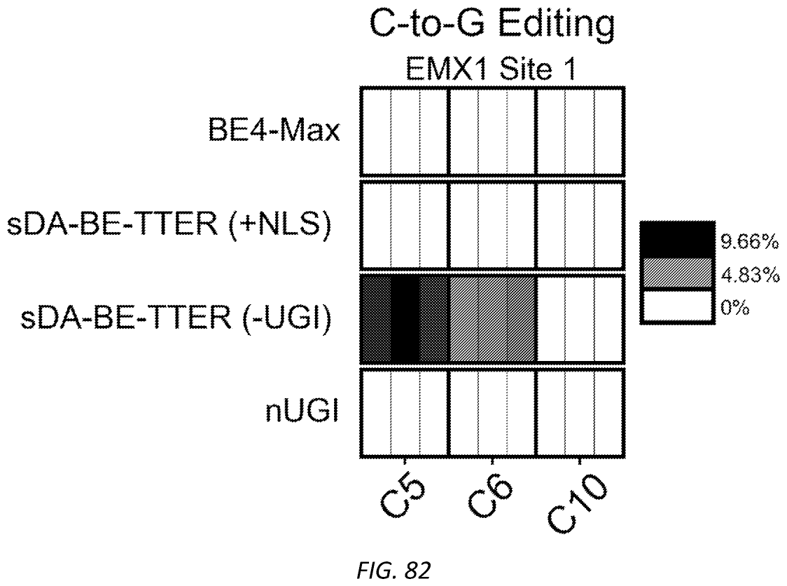

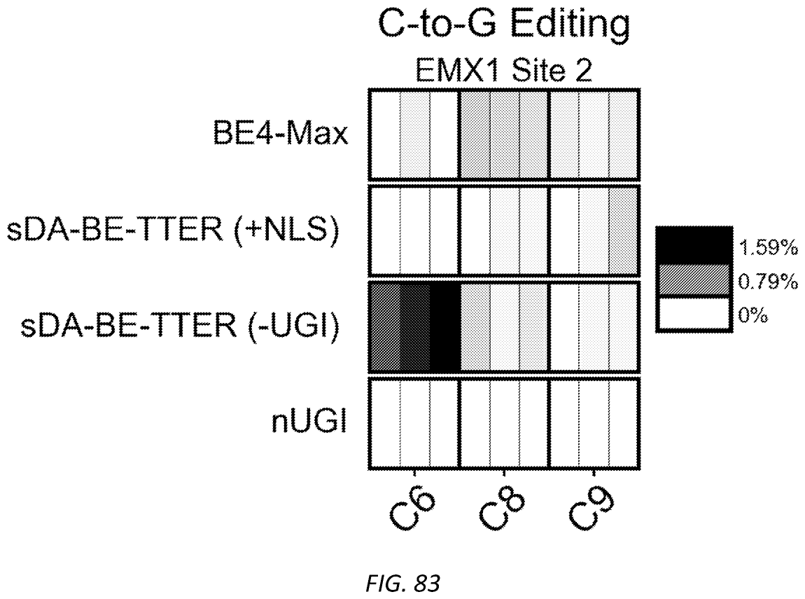

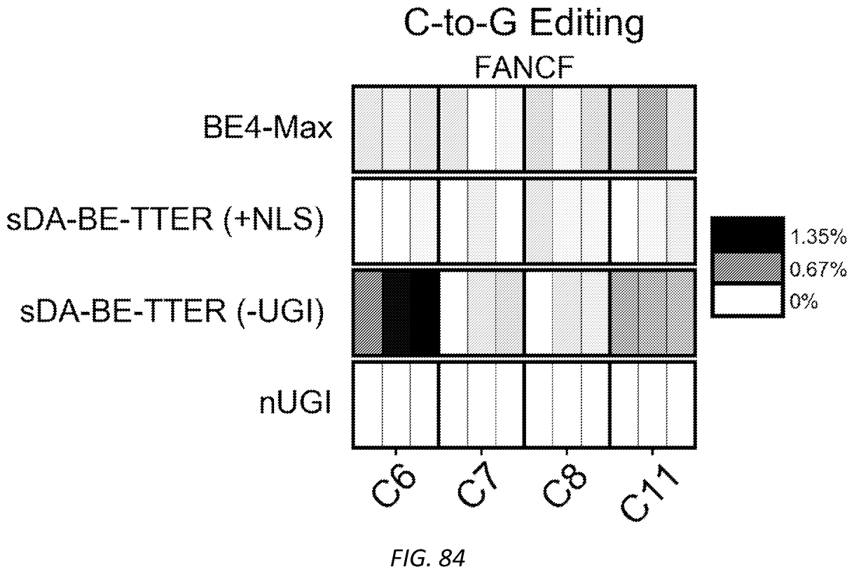

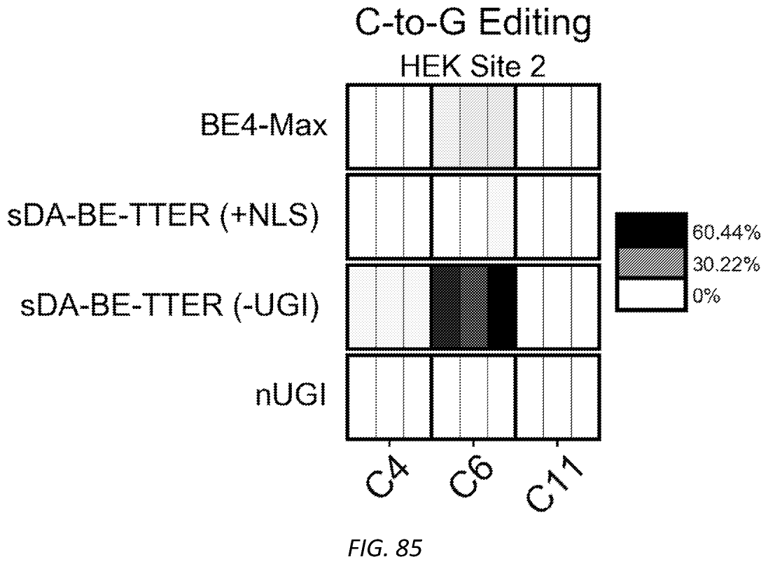

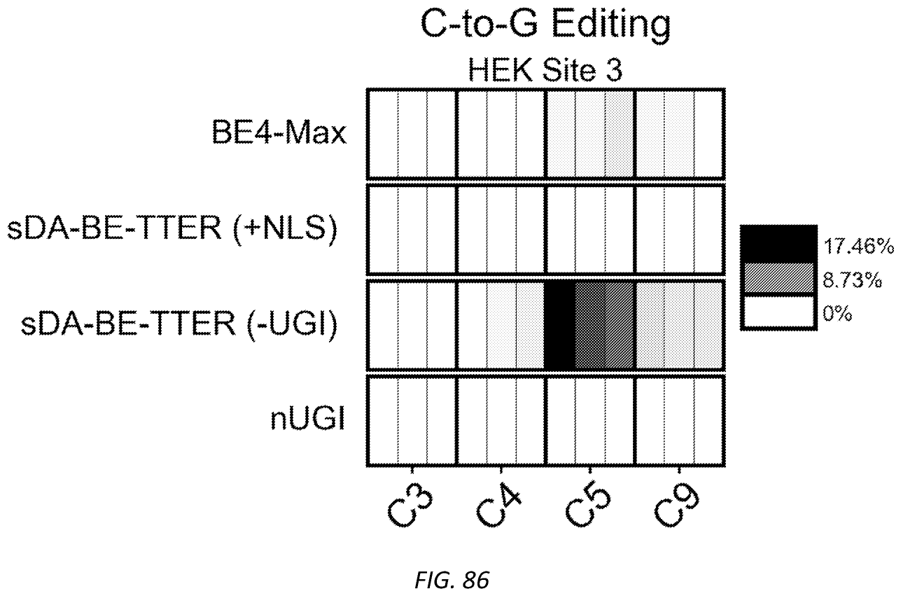

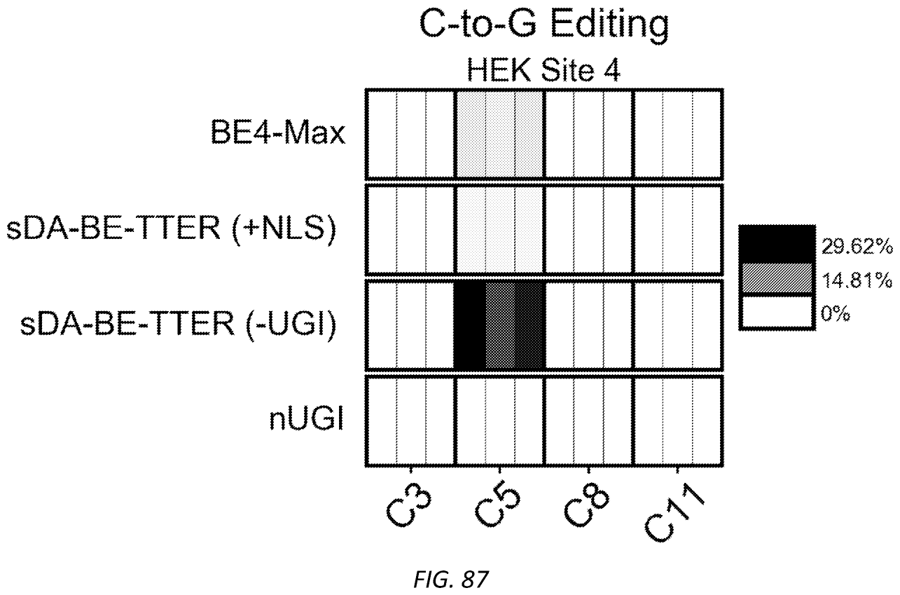

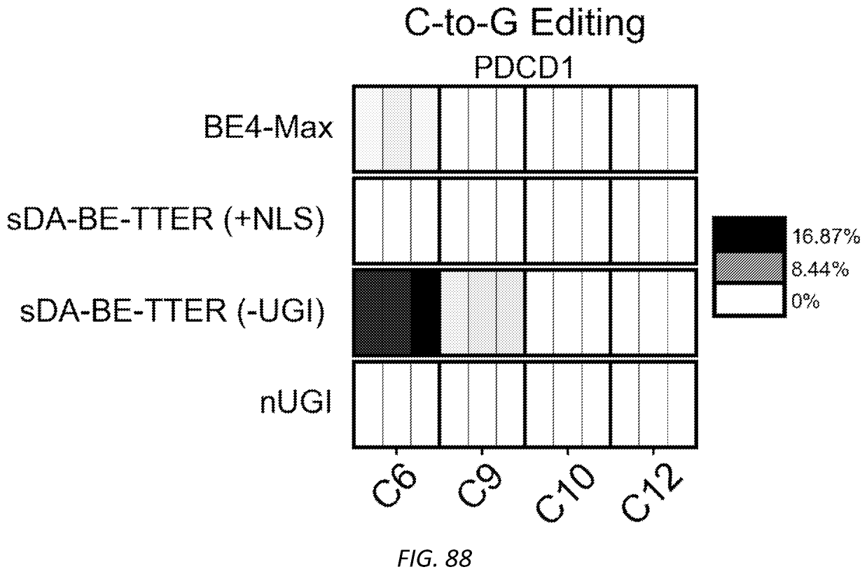

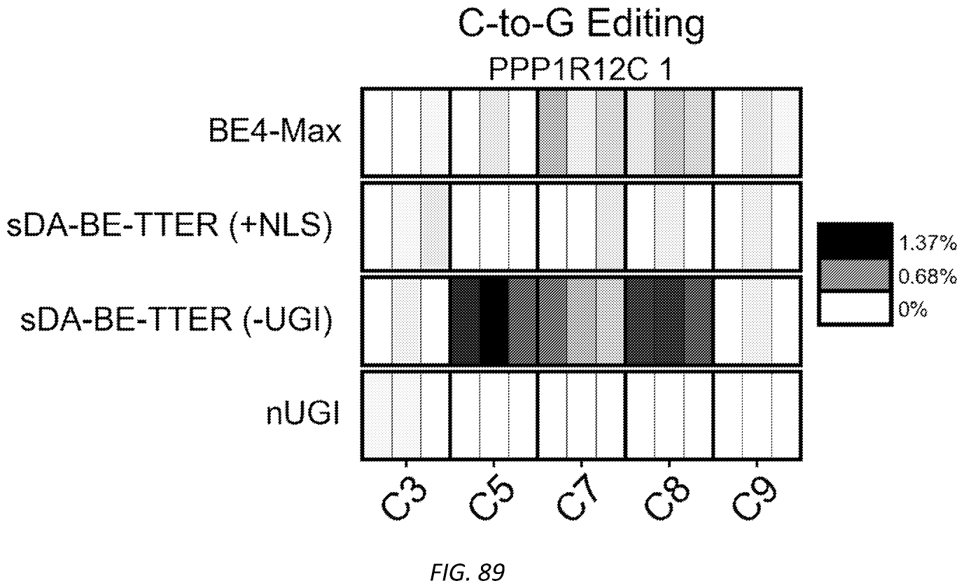

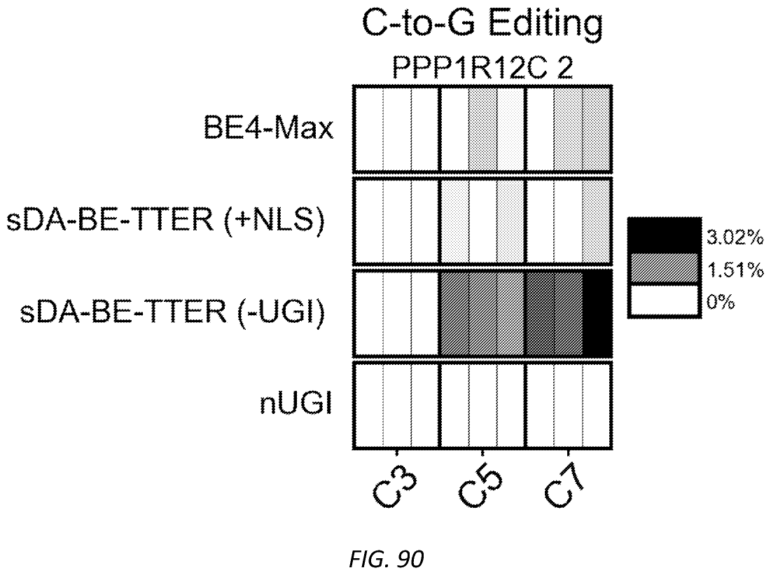

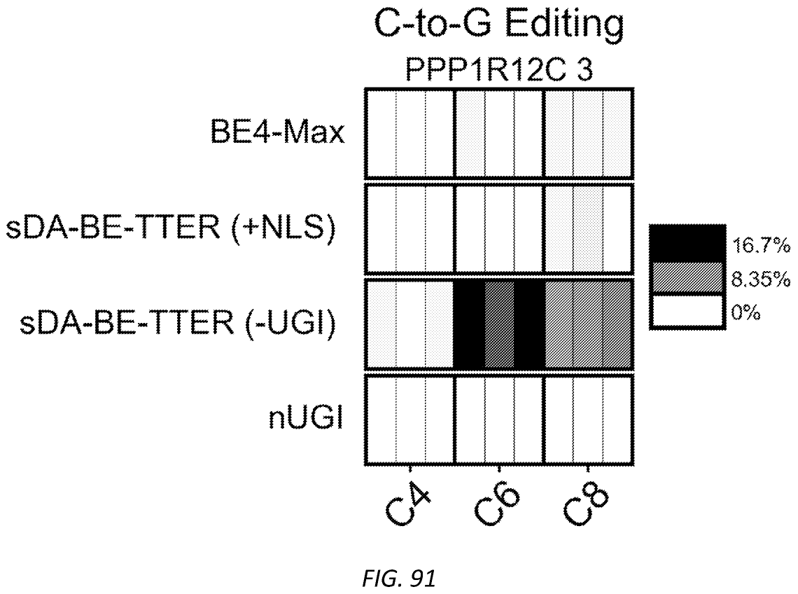

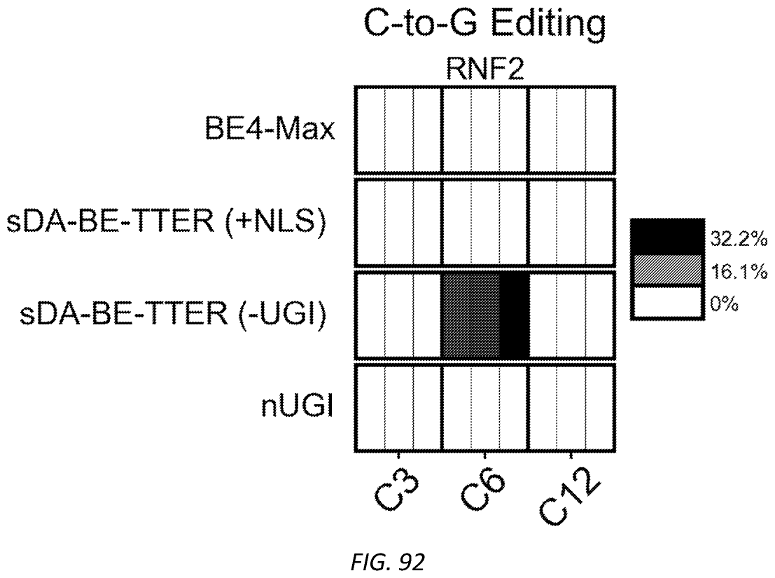

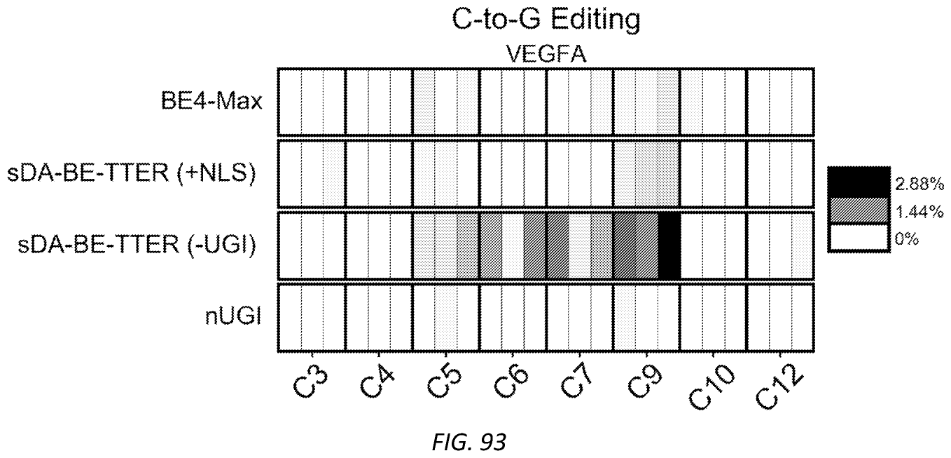

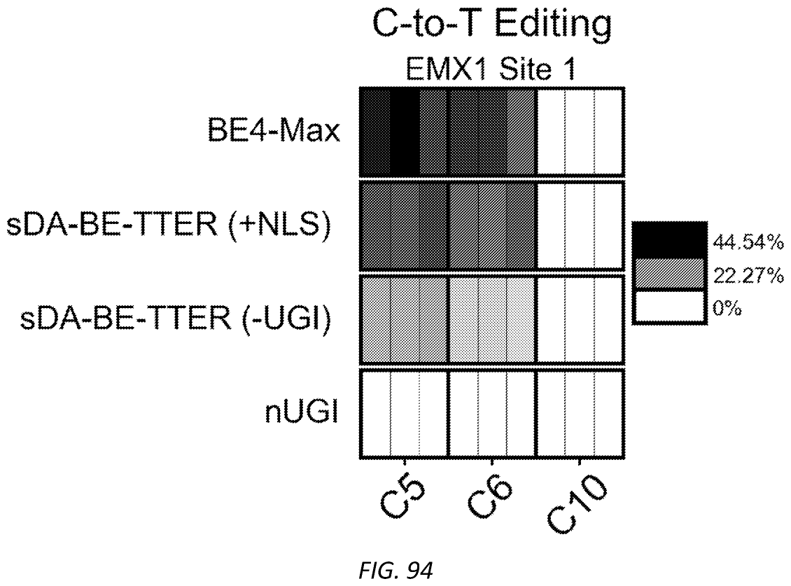

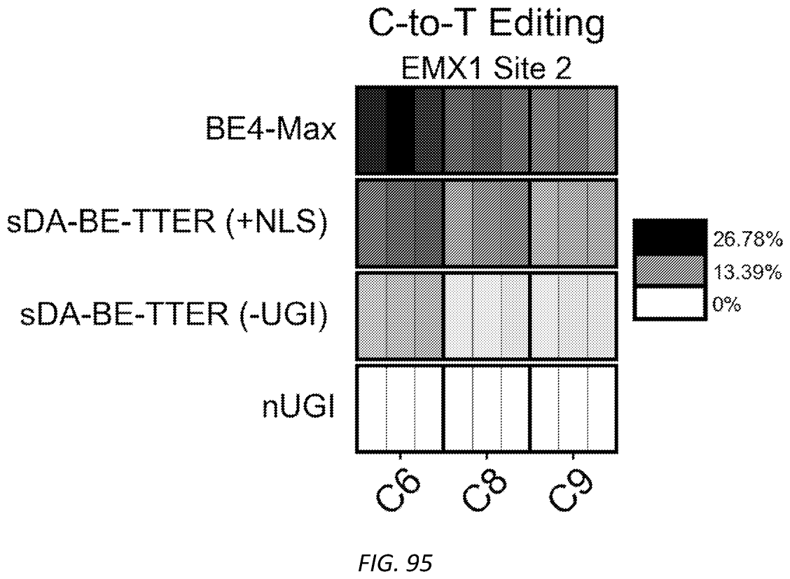

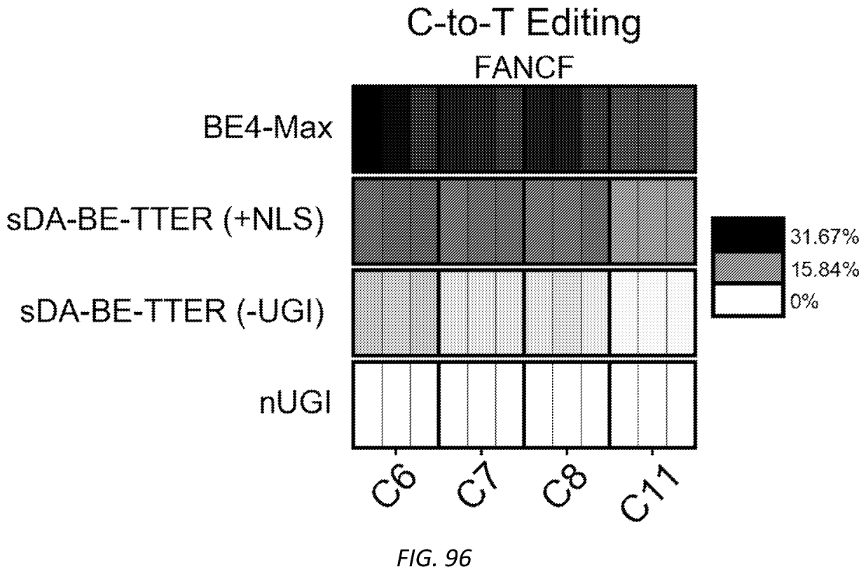

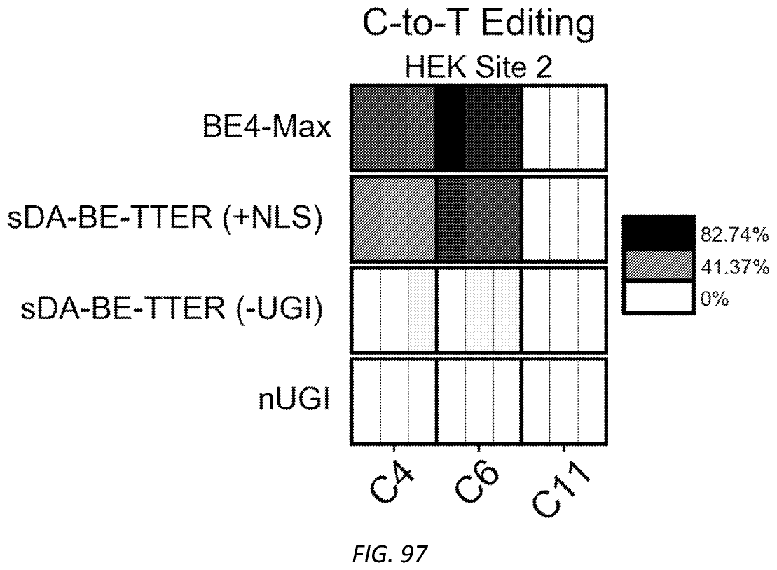

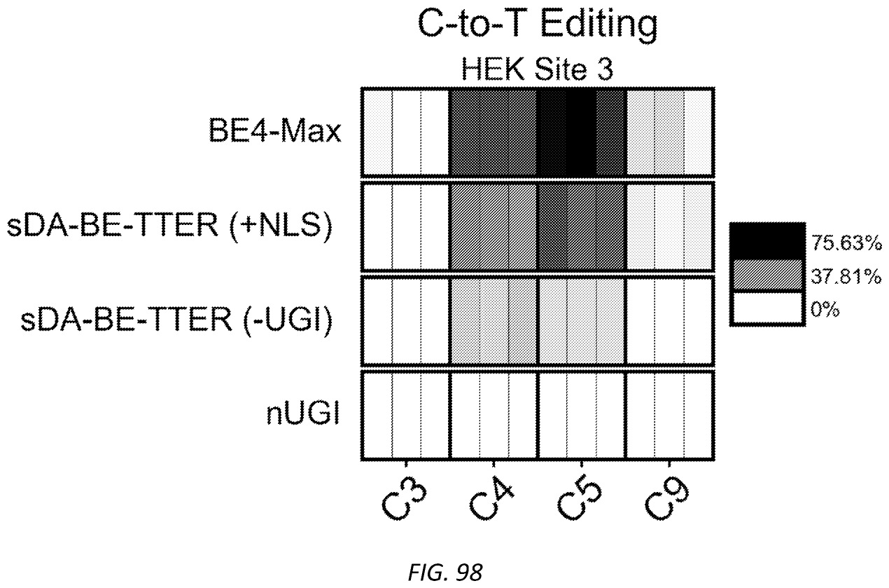

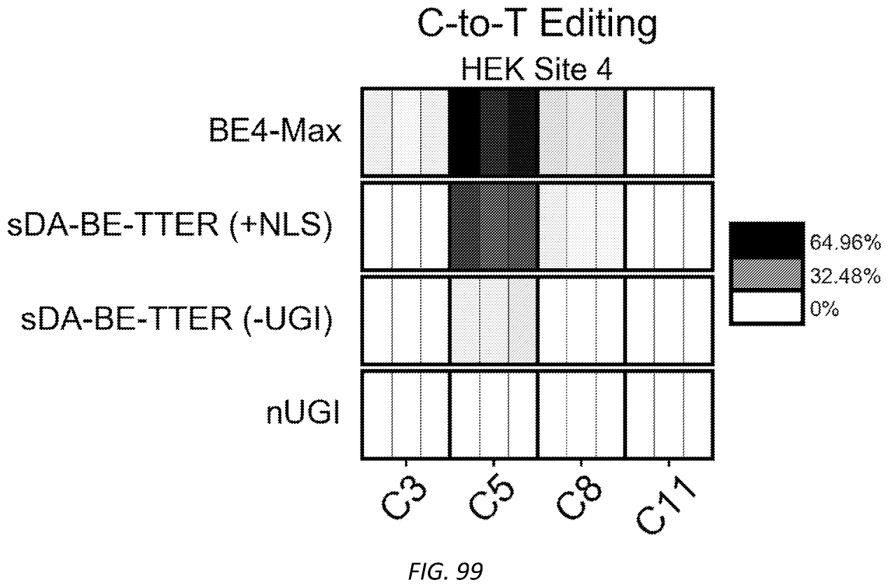

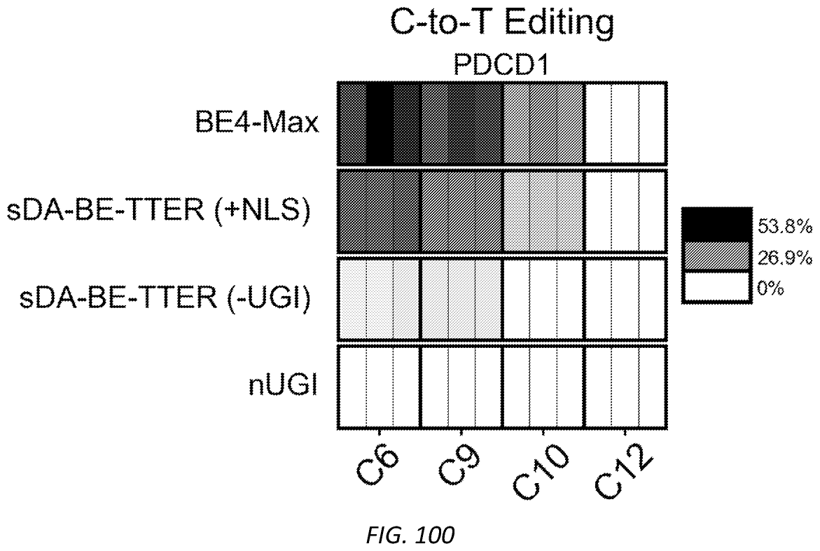

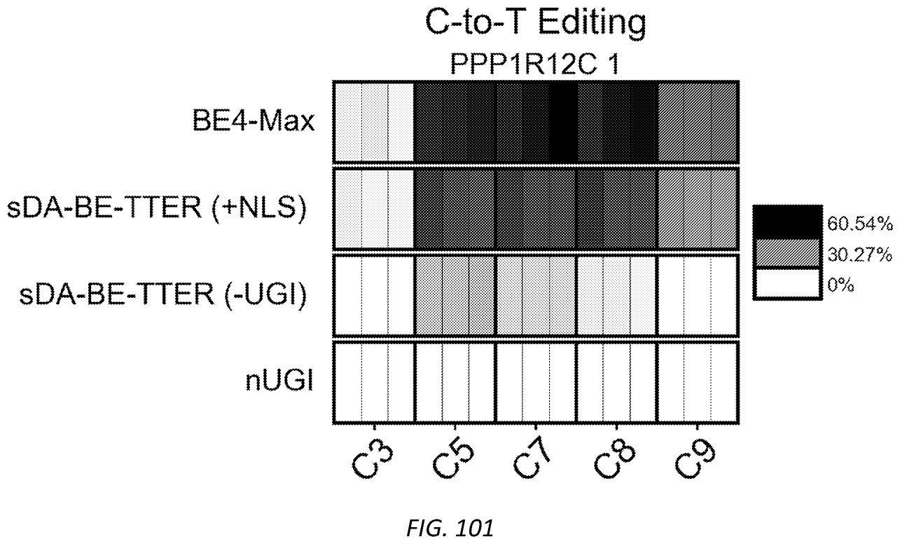

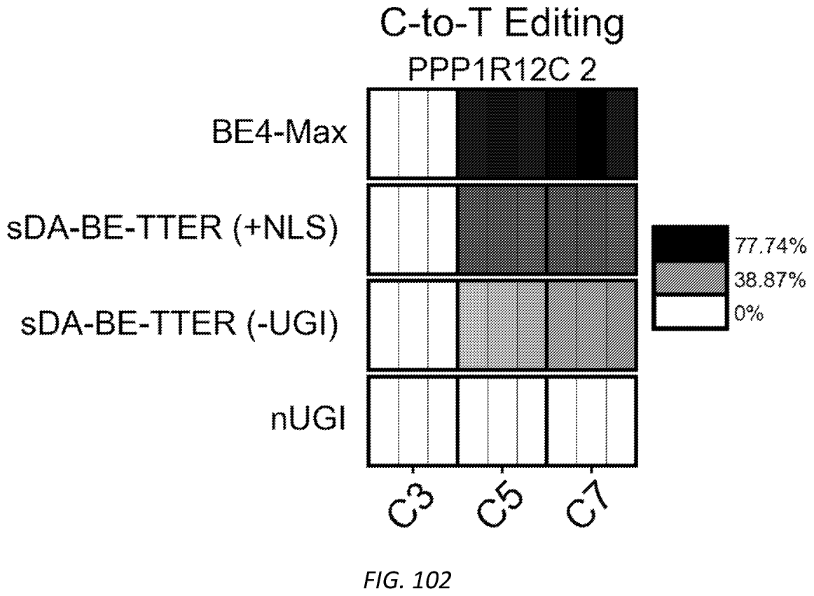

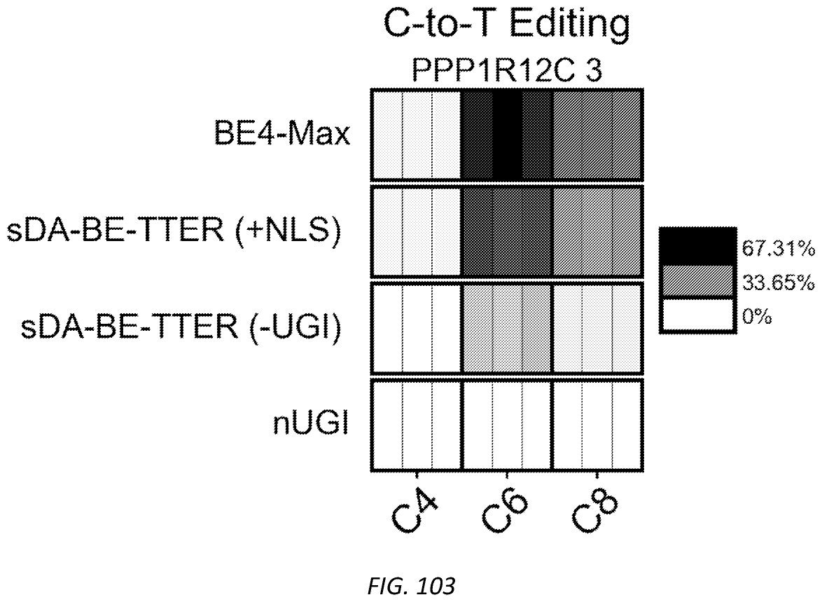

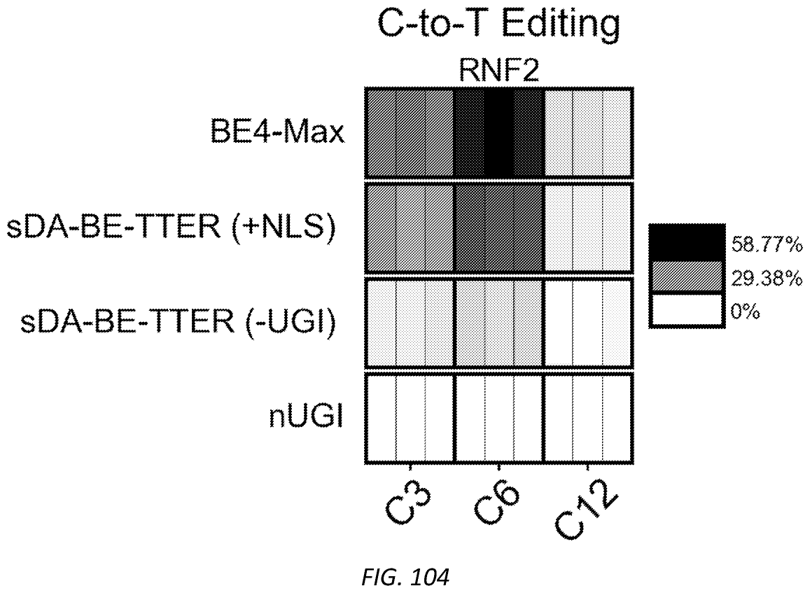

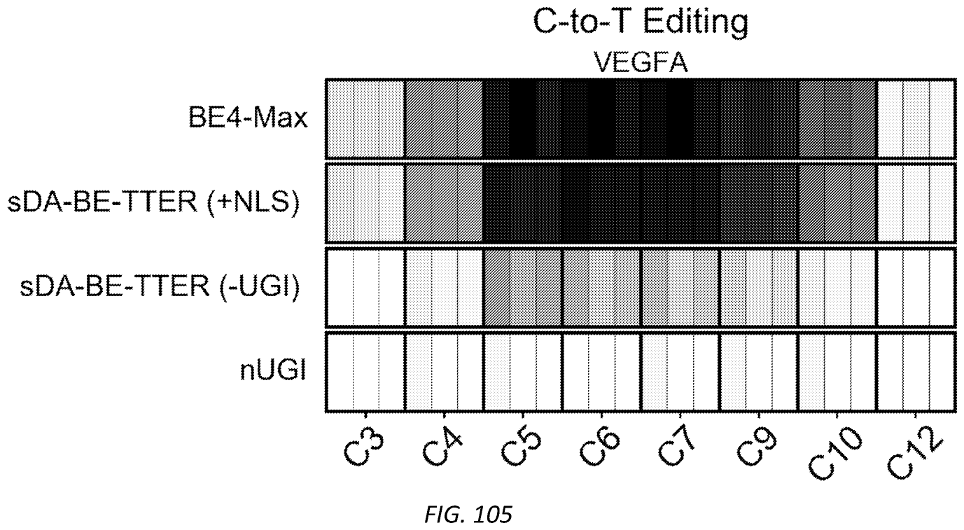

As shown in FIGS. 70-105, we also found that a version of sDA-BE-TTER that lacks any UGI domains (comprising SEQ ID. 9 and SEQ ID. 69) is able to bias editing events from C-to-T to either C-to-A or primarily C-to-G editing, in a manner similar to a recently-described C-to-G Base Editor (CGBE) that comprises a BE4Max architecture in conjunction with a glycosylase domain (Kurt et al., “CRISPR C-to-G base editors for inducing targeted DNA transversions in human cells,” Nat Biotechnol (2020) doi.org/10.1038/s41587-020-0609-x). In our configuration, a UGI-free sDA-BE architecture is able to achieve robust C-to-G or C-to-A editing at some target sites in HEK293T cells without the need for an additional glycosylase domain fused to the architecture, thus potentially expanding its utility to other applications and establishing the capability of sDA-BEs to undergo additional forms of editing events. In addition, we note that sDA-BE-TTER (−UGI) editing events can sometimes result in a mosaic effect, with significant rates of all three C-to-D mutation types existing within the population. Such an outcome may thus enable genetic randomization outcomes if desired. Note that this construct contains an extra C-terminal bipartite NLS in the place of the normal UGI-UGI domain, and is thus shown in comparison to sDA-BE-TTER (+NLS) in FIGS. 70-105.

sDA2.2-BE S83R (-UGI)
(SEQ ID NO: 69)
MGNTRCRITWFLSWSPCGECSRAITEFLSRYPHVTLFIYIARLYHHADPR
NRQGLRDLISSGVTIQIMTEQESGYCWRNFVNYSPSNEAHWPRYPHLWVR
LYVLELYCIILGLPPCLNILRRKQPQLTFFTIALQSCHYQRLPPHILWAT
GLKSGGSSGGSSGSETPGTSESATPESSGGSSGGSDKKYSIGLAIGTNSV
GWAVITDEYKVPSKKFKVLGNTDRHSIKKNLIGALLFDSGETAEATRLKR
TARRRYTRRKNRICYLQEIFSNEMAKVDDSFFHRLEESFLVEEDKKHERH
PIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARL
SKSRRLENLIAQLPGEKKNGLFGNLIALSLGLTPNFKSNFDLAEDAKLQL
SKDTYDDDLDNLLAQIGDQYADLFLAAKNLSDAILLSDILRVNTEITKAP
LSASMIKRYDEHHQDLTLLKALVRQQLPEKYKEIFFDQSKNGYAGYIDGG
ASQEEFYKFIKPILEKMDGTEELLVKLNREDLLRKQRTFDNGSIPHQIHL
GELHAILRRQEDFYPFLKDNREKIEKILTFRIPYYVGPLARGNSRFAWMT
RKSEETITPWNFEEVVDKGASAQSFIERMTNFDKNLPNEKVLPKHSLLYE
YFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDIL
EDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGK
SDNVPSEEVVKKMKNYWRQLLNAKLITQRKFDNLTKAERGGLSELDKAGF
IKRQLVETRQITKHVAQILDSRMNTKYDENDKLIREVKVITLKSKLVSDF
RKDFQFYKVREINNYHHAHDAYLNAVVGTALIKKYPKLESEFVYGDYKVY
DVRKMIAKSEQEIGKATAKYFFYSNIMNFFKTEITLANGEIRKRPLIETN
GETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGI
TIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYL
DEIIEQISEFSKRVILADANLDKVLSAYNKHRDKPIREQAENIIHLFTLT
NLGAPAAFKYFDTTIDRKRYTSTKEVLDATLIHQSITGLYETRIDLSQLG
GDSGGSKRTADGSEFEPKKKRKV

### Exemplary Embodiments

A split-deaminase base-editor (sDA-BE) comprising a bisected BE4-Max (SEQ ID NO:1), with the split site falling generally in the predicted unstructured region between residues T90 and C100 of BE4-Max (corresponding to T72 and C82 in rAPOBEC1), and any derivatives thereof.

A split-deaminase base-editor (sDA-BE) comprising a bisected BE4-Max (SEQ ID NO:1), with one module (sDA1.2) consisting of the amino acids spanning M1 to T90 and a second module (sDA2.2) spanning C100 (with or without an appended N-terminal Met or Met-Gly to create an optimal Kozak sequence) to V1853 (or the C-terminus of any BE4-Max derivative), and any derivatives thereof.

A split-deaminase base-editor (sDA-BE) comprising a bisected BE4-Max (SEQ ID NO:1), with one module (sDA1.2) consisting of the amino acids spanning M1 to T90 or M1 to E91 or M1 to R92, and a second module (sDA2.2) spanning from N97- or T98 or R99 or C100 (with or without an appended N-terminal Met or Met-Gly to create an optimal Kozak sequence) to V1853 (or the C-terminus of any BE4-Max derivative) bearing S83R mutation in rAPOBEC1 (corresponding to S101R in BE4-Max) or any similar mutations, such as S83K or S83H, and any derivatives thereof.

A split-deaminase base-editor (sDA-BE) comprising a bisected BE4-Max with one module (sDA1.2) consisting of the amino acids spanning M1 to T90 or M1 to E91 or M1 to R92, and a second module (sDA2.2) spanning from N97- or T98 or R99 or C100 (with or without an appended N-terminal Met or Met-Gly to create an optimal Kozak sequence) to V1853 (or the C-terminus of any BE4-Max derivative) bearing a K229E mutation in the rAPOBEC1 (corresponding to K247E in BE4-Max) or any similar mutations and any derivatives thereof.

A split-deaminase base-editor (sDA-BE) comprising a bisected BE4-Max (SEQ ID NO:1), with one module (sDA1.2) consisting of the amino acids spanning M1 to T90 or M1 to E91 or M1 to R92, and a second module (sDA2.2) spanning from N97- or T98 or R99 or C100 (with or without an appended N-terminal Met or Met-Gly to create an optimal Kozak sequence) to V1853 (or the C-terminus of any BE4-Max derivative) bearing K229D mutation in rAPOBEC1 (corresponding to K247D in BE4-Max) or any similar mutations and any derivatives thereof.

A split-deaminase base-editor (sDA-BE) comprising a bisected BE4-Max (SEQ ID NO:1), with one module (sDA1.2) consisting of the amino acids spanning M1 to T90 or M1 to E91 or M1 to R92, and a second module (sDA2.2) spanning from N97- or T98 or R99 or C100 (with or without an appended N-terminal Met or Met-Gly to create an optimal Kozak sequence) to V1853 (or the C-terminus of any BE4-Max derivative) bearing a combination of mutations including the rAPOBEC1 K229E mutation (K247E in BE4-Max) or rAPOBEC1 K229D mutation (K247D in BE4-Max) along with the S83R mutation (S101R in BE4-Max) or any similar mutations and any derivatives thereof.

A split-deaminase base-editor (sDA-BE) comprising a bisected BE4-Max (SEQ ID NO:1), with one module (sDA1.2) consisting of the amino acids spanning M1 to T90 (seq 3) or M1 to E91 (seq 4) or M1 to R92 (seq 5), and a second module (sDA2.2) spanning from N97- or T98 or R99 or C100 (with or without an appended N-terminal Met or Met-Gly to create an optimal Kozak sequence) to V1853 (or the C-terminus of any BE4-Max derivative) bearing a combination of mutations including the rAPOBEC1 K229E mutation (K247E in BE4-Max) or rAPOBEC1 K229D mutation (K247D in BE4-Max) along with the S83R mutation (S101R in BE4-Max) or any similar mutations, as well as any combination of the SECURE-Like Ortholog mutations described herein, and any derivatives thereof.

Any of the previously described editors described herein with an extra bipartite NLS inserted into the architecture, characteristically in-between the UGI domains.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A split-deaminase base editor (sDA-BE) comprising:
(i) a first fusion protein comprising:
a first nuclear localization signal (NLS); and
a catalytically inactive or catalytically deficient N-terminal portion of a deaminase enzyme,
but not a programmable DNA binding domain; and
(ii) a second fusion protein comprising:
a second nuclear localization signal (NLS);
a catalytically inactive or catalytically deficient C-terminal portion of the deaminase enzyme; and
a programmable DNA binding domain,
wherein the first fusion protein and second fusion protein, when co-expressed, form a catalytically active deaminase enzyme.

2. The sDA-BE of claim 1, wherein the second fusion protein further comprises an N-terminal methionine.

3. The sDA-BE of claim 1, wherein the second fusion protein further comprises one or more UGI sequences.

4. The sDA-BE of claim 1, wherein the deaminase enzyme is selected from the group consisting of hAID, rAPOBEC1, mAPOBEC3, hAPOBEC3A, hAPOBEC3B, hAPOBEC3C, hAPOBEC3F, hAPOBEC3G, hAPOBEC3H, and variants thereof.

5. The sDA-BE of claim 1, wherein the programmable DNA binding domain is selected from the group consisting of zinc fingers (ZFs), transcription activator effector-like effectors (TALEs), and Clustered Regularly Interspaced Short Palindromic Repeats (CRISPR) Cas RNA-guided nucleases (RGNs), catalytically inactive Cas9 (dCas9) nicking Cas9 (nCas9), and variants thereof.

6. A split-deaminase base editor (sDA-BE) comprising:
(i) a first fusion protein comprising an amino acid sequence selected from the group consisting of amino acids 1-90 of SEQ ID NO:1, amino acids 1-92 of SEQ ID NO:1, SEQ ID NO:8, SEQ ID NO:9; SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, or SEQ ID NO:26; and
(ii) a second fusion protein comprising an amino acid sequence selected from the group consisting of amino acids 101-1,853 of SEQ ID NO:1, amino acids 97-1,853 of SEQ ID NO:1, SEQ ID NO:5, SEQ ID NO:6, SEQ ID NO:7, SEQ ID NO:11, or SEQ ID NO:69.

7. A split-deaminase base editor (sDA-BE) comprising:
(i) a first fusion protein comprising:
a first nuclear localization signal (NLS);
a catalytically inactive or catalytically deficient N-terminal portion of a deaminase enzyme; and
a single strand nickase; and
(ii) a second fusion protein comprising:
a second nuclear localization signal (NLS);
a catalytically inactive or catalytically deficient C-terminal portion of a deaminase enzyme; and
a programmable DNA binding domain,
wherein the first fusion protein and the second fusion protein, when co-expressed, form a catalytically active deaminase enzyme.

8. The sDA-BE of claim 7, wherein the first fusion protein comprises the amino acid sequence of SEQ ID NO:29, and wherein the second fusion protein comprises an amino acid sequence selected from the group consisting of SEQ ID NO:5 and SEQ ID NO:11.

9. A nucleic acid encoding any one or more of the fusion proteins of claim 1.

10. A composition comprising one or more nucleic acids, collectively encoding each of the fusion proteins of the sDA-BE of claim 1.

11. A composition comprising one or more nucleic acid expression vector(s) comprising the nucleic acid(s) of claim 9.

12. A cell comprising one or more nucleic acid expression vector(s) comprising the nucleic acid(s) of claim 9.

13. The cell of claim 11, wherein the cell is an isolated host cell.

14. The cell of claim 11, wherein the cell is a stem cell.

15. The cell of claim 13, wherein the stem cell is a hematopoietic stem cell.

16. A method of targeted deamination of a nucleic acid, the method comprising:
contacting the nucleic acid with the split deaminase base editors (sDA-BE) of claim 1 and, if the programmable DNA binding domain is a CRISPR based programmable DNA binding domain, contacting the nucleic acid with a gRNA.

17. A method of targeted deamination of a nucleic acid in a cell, the method comprising:
expressing the nucleic acid(s) of claim 9 in the cell and, if the programmable DNA binding domain is a CRISPR based programmable DNA binding domain, expressing a gRNA in the cell.

18. The method of claim 17, wherein the cell is a eukaryotic cell.

19. The method of claim 18, wherein the cell is a mammalian cell.

20. The method of claim 19, wherein the cell is a human cell.


SEQUENCE LISTING (112 sequences)

SEQ ID NO: 1 - protein, 1853 aa, Artificial Sequence
  Description of Artificial Sequence: Synthetic polypeptide
MKRTADGSEFESPKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 2 - protein, 90 aa, Artificial Sequence
  Description of Artificial Sequence: Synthetic polypeptide
MKRTADGSEFESPKKKRKVSSETGPVAVDPTLRRRIEPHEFEVFFDPRELRKETCLLYEINWGGRHSIWRHTSQNTNKHVEVNFIEKFTT

SEQ ID NO: 3 - protein, 1755 aa, Artificial Sequence
  Description of Artificial Sequence: Synthetic polypeptide
MCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 4 - protein, 19 aa, Artificial Sequence
  Description of Artificial Sequence: Synthetic peptide
MKRTADGSEFESPKKKRKV

SEQ ID NO: 5 - protein, 1759 aa, Artificial Sequence
  Description of Artificial Sequence: Synthetic polypeptide
MGNTRCRITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 6 - protein, 1759 aa, Artificial Sequence
  Description of Artificial Sequence: Synthetic polypeptide
MGNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLESGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 7 - protein, 1759 aa, Artificial Sequence
  Description of Artificial Sequence: Synthetic polypeptide
MGNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 8 - protein, 91 aa, Artificial Sequence
  Description of Artificial Sequence: Synthetic polypeptide
MKRTADGSEFESPKKKRKVSSETGPVAVDPTLRRRIEPHEFEVFFDPRELRKETCLLYEINWGGRHSIWRHTSQNTNKHVEVNFIEKFTTE

SEQ ID NO: 9 - protein, 92 aa, Artificial Sequence
  Description of Artificial Sequence: Synthetic polypeptide
MKRTADGSEFESPKKKRKVSSETGPVAVDPTLRRRIEPHEFEVFFDPRELRKETCLLYEINWGGRHSIWRHTSQNTNKHVEVNFIEKFTTER

SEQ ID NO: 10 - protein, 90 aa, Artificial Sequence; identical to SEQ ID NO: 2
  Description of Artificial Sequence: Synthetic polypeptide

SEQ ID NO: 11 - protein, 1776 aa, Artificial Sequence
  Description of Artificial Sequence: Synthetic polypeptide
MGNTRCRITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGKRTADGSEFEPKKKRKVGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 12 - protein, 92 aa, Artificial Sequence
  Description of Artificial Sequence: Synthetic polypeptide
MKRTADGSEFESPKKKRKVSSETGPVAVDPTLRRRIEPHEFEVFFDPRELGKETCLLYEINWGGRHSIWRHTSQNTNKHVEVNFIEKFTTER

SEQ ID NO: 13 - protein, 92 aa, Artificial Sequence
  Description of Artificial Sequence: Synthetic polypeptide
MKRTADGSEFESPKKKRKVSSETGPVAVDPTLRRRIEPHEFEVFFDPRELHKETCLLYEINWGGRHSIWRHTSQNTNKHVEVNFIEKFTTER

SEQ ID NO: 14 - protein, 92 aa, Artificial Sequence
  Description of Artificial Sequence: Synthetic polypeptide
MKRTADGSEFESPKKKRKVSSETGPVAVDPTLRRRIEPHEFEVFFDPRELYKETCLLYEINWGGRHSIWRHTSQNTNKHVEVNFIEKFTTER

SEQ ID NO: 15 - protein, 92 aa, Artificial Sequence
  Description of Artificial Sequence: Synthetic polypeptide
MKRTADGSEFESPKKKRKVSSETGPVAVDPTLRRRIEPHEFEVFFDPRELFKETCLLYEINWGGRHSIWRHTSQNTNKHVEVNFIEKFTTER

SEQ ID NO: 16 - protein, 92 aa, Artificial Sequence
  Description of Artificial Sequence: Synthetic polypeptide
MKRTADGSEFESPKKKRKVSSETGPVAVDPTLRRRIEPHEFEVFFDPRELAKETCLLYEINWGGRHSIWRHTSQNTNKHVEVNFIEKFTTER

SEQ ID NO: 17 - protein, 92 aa, Artificial Sequence
  Description of Artificial Sequence: Synthetic polypeptide
MKRTADGSEFESPKKKRKVSSETGPVAVDPTLRRRIEPHEFEVFFDPRELRQETCLLYEINWGGRHSIWRHTSQNTNKHVEVNFIEKFTTER

SEQ ID NO: 18 - protein, 92 aa, Artificial Sequence
  Description of Artificial Sequence: Synthetic polypeptide
MKRTADGSEFESPKKKRKVSSETGPVAVDPTLRRRIEPHEFEVFFDPRELRHETCLLYEINWGGRHSIWRHTSQNTNKHVEVNFIEKFTTER

SEQ ID NO: 19 - protein, 92 aa, Artificial Sequence
  Description of Artificial Sequence: Synthetic polypeptide
MKRTADGSEFESPKKKRKVSSETGPVAVDPTLRRRIEPHEFEVFFDPRELRNETCLLYEINWGGRHSIWRHTSQNTNKHVEVNFIEKFTTER

SEQ ID NO: 20 - protein, 92 aa, Artificial Sequence
  Description of Artificial Sequence: Synthetic polypeptide
MKRTADGSEFESPKKKRKVSSETGPVAVDPTLRRRIEPHEFEVFFDPRELRKETCLLYEINWGGRHSIWRHTSQNTNKHVEVNFICKFTTER

SEQ ID NO: 21 - protein, 92 aa, Artificial Sequence
  Description of Artificial Sequence: Synthetic polypeptide
MKRTADGSEFESPKKKRKVSSETGPVAVDPTLRRRIEPHEFEVFFDPRELRKETCLLYEINWGGRHSIWRHTSQNTNKHVEVNFIKKFTTER

SEQ ID NO: 22 - protein, 92 aa, Artificial Sequence
  Description of Artificial Sequence: Synthetic polypeptide
MKRTADGSEFESPKKKRKVSSETGPVAVDPTLRRRIEPHEFEVFFDPRELRKETCLLYEINWGGRHSIWRHTSQNTNKHVEVNFIQKFTTER

SEQ ID NO: 23 - protein, 92 aa, Artificial Sequence
  Description of Artificial Sequence: Synthetic polypeptide
MKRTADGSEFESPKKKRKVSSETGPVAVDPTLRRRIEPHEFEVFFDPRELRKETCLLYEINWGGRHSIWRHTSQNTNKHVEVNFIWKFTTER

SEQ ID NO: 24 - protein, 92 aa, Artificial Sequence
  Description of Artificial Sequence: Synthetic polypeptide
MKRTADGSEFESPKKKRKVSSETGPVAVDPTLRRRIEPHEFEVFFDPRELRKETCLLYEINWGGRHSIWRHTSQNTNKHVEVNFIHKFTTER

SEQ ID NO: 25 - protein, 92 aa, Artificial Sequence
  Description of Artificial Sequence: Synthetic polypeptide
MKRTADGSEFESPKKKRKVSSETGPVAVDPTLRRRIEPHEFEVFFDPRELRKETCLLYEINWGGRHSIWRHTSQNTNKHVEVNFIDKFTTER

SEQ ID NO: 26 - protein, 92 aa, Artificial Sequence
  Description of Artificial Sequence: Synthetic polypeptide
MKRTADGSEFESPKKKRKVSSETGPVAVDPTLRRRIEPHEFEVFFDPRELRKETCLLYEINWGGRHSIWRHTSQNTNKHVEVNFIRKFTTER

SEQ ID NO: 27 - DNA, 6 bp, Unknown
  Description of Unknown: PAM sequence
  modified_base (1)..(3): a, c, t, g, unknown or other
nnnrrt

SEQ ID NO: 28 - skipped

SEQ ID NO: 29 - protein, 1150 aa, Artificial Sequence
  Description of Artificial Sequence: Synthetic polypeptide
MGKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKGGSGGSKRTADGSEFESPKKKRKVSSETGPVAVDPTLRRRIEPHEFEVFFDPRELRKETCLLYEINWGGRHSIWRHTSQNTNKHVEVNFIEKFTTER

SEQ ID NO: 30 - DNA, 22 bp, Unknown
  Description of Unknown: PPP1R12C 1 target sequence
gggattggaatgccggggcggg

SEQ ID NO: 31 - DNA, 20 bp, Unknown
  Description of Unknown: PPP1R12C 1 target sequence
ggaatgccggggcggggtgg

SEQ ID NO: 32 - DNA, 22 bp, Unknown
  Description of Unknown: PPP1R12C 1 target sequence
gtaggattgctggaaccctgcc

SEQ ID NO: 33 - DNA, 22 bp, Unknown
  Description of Unknown: PPP1R12C 1 target sequence
gaaagacctgcggcagggttcc

SEQ ID NO: 34 - DNA, 19 bp, Unknown
  Description of Unknown: PPP1R12C 1 target sequence
gccgcaggtctttctggga

SEQ ID NO: 35 - DNA, 22 bp, Unknown
  Description of Unknown: PPP1R12C 1 target sequence
gaggggatgcgtttacttgggg

SEQ ID NO: 36 - DNA, 19 bp, Unknown
  Description of Unknown: PPP1R12C 1 target sequence
gccccggcattccaatccc

SEQ ID NO: 37 - DNA, 22 bp, Unknown
  Description of Unknown: PPP1R12C 1 target sequence
cccacccaccccgccccggcat

SEQ ID NO: 38 - DNA, 17 bp, Unknown
  Description of Unknown: PPP1R12C 1 target sequence
ccaccccgccccggcat

SEQ ID NO: 39 - DNA, 26 bp, Unknown
  Description of Unknown: PDCD1 target sequence
cattccggaatgccggggcggggtgg

SEQ ID NO: 40 - DNA, 17 bp, Unknown
  Description of Unknown: PDCD1 target sequence
gtcctggccgggctggc

SEQ ID NO: 41 - DNA, 18 bp, Unknown
  Description of Unknown: PDCD1 target sequence
gttgtgtgacacggaagc

SEQ ID NO: 42-46 - skipped (5 entries)

SEQ ID NO: 47 - protein, 83 aa, Unknown
  Description of Unknown: Bacillus phage PBS2 sequence
TNLSDIIEKETGKQLVIQESILMLPEEVEEVIGNKPESDILVHTAYDESTDENVMLLTSDAPEYKPWALVIQDSNGENKIKML

SEQ ID NO: 48 - protein, 7 aa, Artificial Sequence
  Description of Artificial Sequence: Synthetic peptide
PKKKRRV

SEQ ID NO: 49 - protein, 16 aa, Artificial Sequence
  Description of Artificial Sequence: Synthetic peptide
KRPAATKKAGQAKKKK

SEQ ID NO: 50 - protein, 1368 aa, Streptococcus pyogenes [sequence omitted]

SEQ ID NO: 51 - DNA, 262 bp, Artificial Sequence
  Description of Artificial Sequence: Synthetic polynucleotide
  Description of Combined DNA/RNA Molecule: Synthetic polynucleotide
  modified_base (1)..(20): a, c, t, or g
  misc_feature (1)..(20): This region may encompass 17-20 nucleotides
  modified_base (63)..(262): a, c, u, g, unknown or other
  misc_feature (63)..(262): This region may encompass 0-200 nucleotides
nnnnnnnnnnnnnnnnnnnnguuuuagagcuagaaauagcaaguuaaaauaaggcuaguccgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 52 - DNA, 275 bp, Artificial Sequence
  Description of Artificial Sequence: Synthetic polynucleotide
  Description of Combined DNA/RNA Molecule: Synthetic polynucleotide
  modified_base (1)..(20): a, c, t, or g
  misc_feature (1)..(20): This region may encompass 17-20 nucleotides
  modified_base (76)..(275): a, c, u, g, unknown or other
  misc_feature (76)..(275): This region may encompass 0-200 nucleotides
nnnnnnnnnnnnnnnnnnnnguuuuagagcuaugcugaaaagcauagcaaguuaaaauaaggcuaguccguuaucnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 53 - DNA, 287 bp, Artificial Sequence
  Description of Artificial Sequence: Synthetic polynucleotide
  Description of Combined DNA/RNA Molecule: Synthetic polynucleotide
  modified_base (1)..(20): a, c, t, or g
  misc_feature (1)..(20): This region may encompass 17-20 nucleotides
  modified_base (88)..(287): a, c, u, g, unknown or other
  misc_feature (88)..(287): This region may encompass 0-200 nucleotides
nnnnnnnnnnnnnnnnnnnnguuuuagagcuaugcuguuuuggaaacaaaacagcauagcaaguuaaaauaaggcuaguccguuaucnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 54 - DNA, 296 bp, Artificial Sequence
  Description of Artificial Sequence: Synthetic polynucleotide
  Description of Combined DNA/RNA Molecule: Synthetic polynucleotide
  modified_base (1)..(20): a, c, t, or g
  misc_feature (1)..(20): This region may encompass 17-20 nucleotides
  modified_base (97)..(296): a, c, u, g, unknown or other
  misc_feature (97)..(296): This region may encompass 0-200 nucleotides
nnnnnnnnnnnnnnnnnnnnguuuuagagcuagaaauagcaaguuaaaauaaggcuaguccguuaucaacuugaaaaaguggcaccgagucggugcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 55 - DNA, 96 bp, Artificial Sequence
  Description of Artificial Sequence: Synthetic oligonucleotide
  Description of Combined DNA/RNA Molecule: Synthetic oligonucleotide
  modified_base (1)..(20): a, c, t, or g
  misc_feature (1)..(20): This region may encompass 17-20 nucleotides
nnnnnnnnnnnnnnnnnnnnguuuaagagcuagaaauagcaaguuuaaauaaggcuaguccguuaucaacuugaaaaaguggcaccgagucggugc

SEQ ID NO: 56 - DNA, 106 bp, Artificial Sequence
  Description of Artificial Sequence: Synthetic polynucleotide
  Description of Combined DNA/RNA Molecule: Synthetic polynucleotide
  modified_base (1)..(20): a, c, t, or g
  misc_feature (1)..(20): This region may encompass 17-20 nucleotides
nnnnnnnnnnnnnnnnnnnnguuuuagagcuaugcuggaaacagcauagcaaguuuaaauaaggcuaguccguuaucaacuugaaaaaguggcaccgagucggugc

SEQ ID NO: 57 - DNA, 106 bp, Artificial Sequence
  Description of Artificial Sequence: Synthetic polynucleotide
  Description of Combined DNA/RNA Molecule: Synthetic polynucleotide
  modified_base (1)..(20): a, c, t, or g
  misc_feature (1)..(20): This region may encompass 17-20 nucleotides
nnnnnnnnnnnnnnnnnnnnguuuaagagcuaugcuggaaacagcauagcaaguuuaaauaaggcuaguccguuaucaacuugaaaaaguggcaccgagucggugc

SEQ ID NO: 58 - DNA, 32 bp, Artificial Sequence
  Description of Artificial Sequence: Synthetic oligonucleotide
  Description of Combined DNA/RNA Molecule: Synthetic oligonucleotide
  modified_base (1)..(20): a, c, t, or g
  misc_feature (1)..(20): This region may encompass 17-20 nucleotides
nnnnnnnnnnnnnnnnnnnnguuuuagagcua

SEQ ID NO: 59 - DNA, 42 bp, Artificial Sequence
  Description of Artificial Sequence: Synthetic oligonucleotide
  Description of Combined DNA/RNA Molecule: Synthetic oligonucleotide
  modified_base (1)..(20): a, c, t, or g
  misc_feature (1)..(20): This region may encompass 17-20 nucleotides
nnnnnnnnnnnnnnnnnnnnguuuuagagcuaugcuguuuug

SEQ ID NO: 60 - DNA, 36 bp, Artificial Sequence
  Description of Artificial Sequence: Synthetic oligonucleotide
  Description of Combined DNA/RNA Molecule: Synthetic oligonucleotide
  modified_base (1)..(20): a, c, t, or g
  misc_feature (1)..(20): This region may encompass 17-20 nucleotides
nnnnnnnnnnnnnnnnnnnnguuuuagagcuaugcu

SEQ ID NO: 61 - RNA, 79 nt, Artificial Sequence
  Description of Artificial Sequence: Synthetic oligonucleotide
ggaaccauucaaaacagcauagcaaguuaaaauaaggcuaguccguuaucaacuugaaaaaguggcaccgagucggugc

SEQ ID NO: 62 - RNA, 60 nt, Artificial Sequence
  Description of Artificial Sequence: Synthetic oligonucleotide
uagcaaguuaaaauaaggcuaguccguuaucaacuugaaaaaguggcaccgagucggugc

SEQ ID NO: 63 - RNA, 64 nt, Artificial Sequence
  Description of Artificial Sequence: Synthetic oligonucleotide
agcauagcaaguuaaaauaaggcuaguccguuaucaacuugaaaaaguggcaccgagucggugc

SEQ ID NO: 64 - DNA, 42 bp, Artificial Sequence; identical to SEQ ID NO: 59
  Description of Artificial Sequence: Synthetic oligonucleotide
  Description of Combined DNA/RNA Molecule: Synthetic oligonucleotide
  modified_base (1)..(20): a, c, t, or g
  misc_feature (1)..(20): This region may encompass 17-20 nucleotides

SEQ ID NO: 65 - RNA, 79 nt, Artificial Sequence; identical to SEQ ID NO: 61
  Description of Artificial Sequence: Synthetic oligonucleotide

SEQ ID NO: 66 - DNA, 32 bp, Artificial Sequence; identical to SEQ ID NO: 58
  Description of Artificial Sequence: Synthetic oligonucleotide
  Description of Combined DNA/RNA Molecule: Synthetic oligonucleotide
  modified_base (1)..(20): a, c, t, or g
  misc_feature (1)..(20): This region may encompass 17-20 nucleotides

SEQ ID NO: 67 - DNA, 36 bp, Artificial Sequence; identical to SEQ ID NO: 60
  Description of Artificial Sequence: Synthetic oligonucleotide
  Description of Combined DNA/RNA Molecule: Synthetic oligonucleotide
  modified_base (1)..(20): a, c, t, or g
  misc_feature (1)..(20): This region may encompass 17-20 nucleotides

SEQ ID NO: 68 - RNA, 64 nt, Artificial Sequence; identical to SEQ ID NO: 63
  Description of Artificial Sequence: Synthetic oligonucleotide

SEQ ID NO: 69 - protein, 1573 aa, Artificial Sequence
  Description of Artificial Sequence: Synthetic polypeptide
MGNTRCRITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKV

SEQ ID NO: 70 - DNA, 8961 bp, Artificial Sequence
  Description of Artificial Sequence: Synthetic polynucleotide
atatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatccgcggccgctaatacgactcactatagggagagccgccaccatgaaacggacagccgacggaagcgagttcgagtcaccaaagaagaagcggaaagtctcctcagagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccaataccagatgtagcatcacatggtttctgagctggtccccttgcggagagtgtagcagggccatcaccgagttcctgtccagatatccacacgtgacactgtttatctacatcgccaggctgtatcaccacgcagacccaaggaataggcagggcctgcgcgatctgatcagctccggcgtgaccatccagatcatgacagagcaggagtccggctactgctggcggaacttcgtgaattattctcctagcaacgaggcccactggcctaggtacccacacctgtgggtgcgcctgtacgtgctggagctgtattgcatcatcctgggcctgcccccttgtctgaatatcctgcggagaaagcagccccagctgaccttctttacaatcgccctgcagtcttgtcactatcagaggctgccaccccacatcctgtgggccacaggcctgaagtctggaggatctagcggaggatcctctggcagcgagacaccaggaacaagcgagtcagcaacaccagagagcagtggcggcagcagcggcggcagcgacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtctaaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgataccgtcgacctctagctagagcttggcgtaatcatggtcatagctgtttcctgtgtgaaattgttatccgctcacaattccacacaacatacgagccggaagcataaagtgtaaagcctagggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacactcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcgatctcccgatcccctagggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatc

SEQ ID NO: 71 - DNA, 8961 bp, Artificial Sequence
  Description of Artificial Sequence: Synthetic polynucleotide
gatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtcgaccctaggggatcgggagatcgatctcccgatccgtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagtgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccctaggctttacactttatgcttccggctcgtatgttgtgtggaattgtgagcggataacaatttcacacaggaaacagctatgaccatgattacgccaagctctagctagaggtcgacggtatcgagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaactcaatggtgatggtgatgatgaccggttagactttcctcttcttcttgggctcgaattcgctgccgtcggcggttctttttgagccgccagacagcattttgattttgttctctccgttggaatcctggatgaccagagcccagggcttatactctggggcgtcggatgtcagcagcatcacattttcgtcggtactctcgtcgtaggcggtatggaccaggatatcgctctcaggcttgtttccgatcacttcttcgacttcttcgggcagcatcaggatgctctcctggatgaccagctgcttgcctgtctccttctcgatgatgtcagacaggttggtgctgcctccagatcctccggatcctccgctcagcatcttgatcttattctcgccgttagaatcctggatgaccagggcccaaggcttatactcgggggcgtcagaggtcagcagcatcacattctcatctgtggactcgtcgtaggcggtgtgcaccaggatgtcagactctggcttgttgccgatcacttcctccacctcctcaggcagcatcaggatggactcctgaatgaccagctgtttcccagtctccttctcaatgatgtcgctcagattagtgctccccccgctcccgccgctcccgccgctgtcacctcccagctgagacaggtcgatccgtgtctcgtacaggccggtgatgctctggtggatcagggtggcgtccagcacctctttggtgctggtgtacctcttccggtcgatggtggtgtcaaagtacttgaaggcggcaggggctcccagattggtcagggtaaacaggtggatgatattctcggcctgctctctgatgggcttatcccggtgcttgttgtaggcggacagcactttgtccagattagcgtcggccaggatcactctcttggagaactcgctgatctgctcgatgatctcgtccaggtagtgcttgtgctgttccacaaacagctgtttctgctcattatcctcgggggagcccttcagcttctcatagtggctggccaggtacaggaagttcacatatttggagggcagggccagttcgtttcccttctgcagttcgccggcagaggccagcattctcttccggccgttttccagctcgaacagggagtacttaggcagcttgatgatcaggtcctttttcacttctttgtagcccttggcttccagaaagtcgatgggattcttctcgaagctgcttctttccatgatggtgatccccagcagctctttcacactcttcagtttcttggacttgcccttttccactttggccaccaccagcacagaataggccacggtggggctgtcgaagccgccgtacttcttagggtcccagtccttctttctggcgatcagcttatcgctgttcctcttgggcaggatagactctttgctgaagccgcctgtctgcacctcggtctttttcacgatattcacttggggcatgctcagcactttccgcacggtggcaaaatcccggcccttatcccacacgatctccccggtttcgccgtttgtctcgatcagaggccgcttccggatctcgccgttggccagggtaatctcggtcttgaaaaagttcatgatgttgctgtagaagaagtacttggcggtagccttgccgatttcctgctcgctcttggcgatcatcttccgcacgtcgtacaccttgtagtcgccgtacacgaactcgctttccagcttagggtactttttgatcagggcggttcccacgacggcgttcaggtaggcgtcgtgggcgtggtggtagttgttgatctcgcgcactttgtaaaactggaaatccttccggaaatcggacaccagcttggacttcagggtgatcactttcacttcccggatcagcttgtcattctcgtcgtacttagtgttcatccgggagtccaggatctgtgccacgtgctttgtgatctgccgggtttccaccagctgtctcttgatgaagccggccttatccagttcgctcaggccgcctctctcggccttggtcagattgtcgaactttctctgggtaatcagcttggcgttcagcagctgccgccagtagttcttcatcttcttcacgacctcttcggagggcacgttgtcgctcttgccccggttcttgtcgcttctggtcagcaccttgttgtcgatggagtcgtccttcagaaagctctgaggcacgatatggtccacatcgtagtcggacagccggttgatgtccagttcctggtccacgtacatatcccgcccattctgcaggtagtacaggtacagcttctcgttctgcagctgggtgttttccacggggtgttctttcaggatctggctgcccagctctttgatgccctcttcgatccgcttcattctctcgcggctgttcttctgtcccttctgggtggtctggttctctctggccatttcgatcacgatgttctcgggcttgtgccggcccatcactttcacgagctcgtccaccaccttcactgtctgcaggatgcccttcttaatggcggggctgccggccagattggcaatgtgctcgtgcaggctatcgccctggccggacacctgggctttctggatgtcctctttaaaggtcaggctgtcgtcgtggatcagctgcatgaagtttctgttggcgaagccgtcggacttcaggaaatccaggattgtcttgccggactgcttgtcccggatgccgttgatcagcttccggctcagcctgccccagccggtgtatctccgccgcttcagctgcttcatcactttgtcgtcgaacaggtgggcataggttttcagccgttcctcgatcatctctctgtcctcaaacagtgtcagggtcagcacgatatcttccagaatgtcctcgttttcctcattgtccaggaagtccttgtccttgataattttcagcagatcgtggtatgtgcccagggaggcgttgaaccgatcttccacgccggagatttccacggagtcgaagcactcgattttcttgaagtagtcctctttcagctgcttcacggtcactttccggttggtcttgaacagcaggtccacgatggcctttttctgctcgccgctcaggaaggcgggctttctcattccctcggtcacgtatttcactttggtcagctcgttatacacggtgaagtactcgtacagcaggctgtgcttgggcagcaccttctcgttgggcaggttcttatcgaagttggtcatccgctcgatgaagctctgggcggaagcgcccttgtccaccacttcctcgaagttccagggggtgatggtttcctcgctctttctggtcatccaggcgaatctgctgtttcccctggccagagggcccacgtagtaggggatgcggaaggtcaggatcttctcgatcttttcccggttgtccttcaggaatgggtaaaaatcttcctgccgccgcagaatggcgtgcagctctcccaggtggatctggtgggggatgctgccgttgtcgaaggtccgctgcttccgcagcaggtcctctctgttcagcttcacgagcagttcctcggtgccgtccatcttttccaggatgggcttgatgaacttgtagaactcttcctggctggctccgccgtcaatgtagccggcgtagccgttcttgctctggtcgaagaaaatctctttgtacttctcaggcagctgctgccgcacgagagctttcagcagggtcaggtcctggtggtgctcgtcgtatctcttgatcatagaggcgctcaggggggccttggtgatctcggtgttcactctcaggatgtcgctcagcaggatggcgtcggacaggttcttggcggccagaaacaggtcggcgtactggtcgccgatctgggccagcaggttgtccaggtcgtcgtcgtaggtgtccttgctcagctgcagtttggcatcctcggccaggtcgaagttgctcttgaagttgggggtcaggcccaggctcagggcaatcaggtttccgaacaggccattcttcttctcgccgggcagctgggcgatcagattttccagccgtctgctcttgctcagtctggcagacaggatggccttggcgtccacgccgctggcgttgatggggttttcctcgaacagctggttgtaggtctgcaccagctggatgaacagcttgtccacgtcgctgttgtcggggttcaggtcgccctcgatcaggaagtggccccggaacttgatcatgtgggccagggccagatagatcagccgcaggtcggccttgtcggtgctgtccaccagtttctttctcaggtggtagatggtggggtacttctcgtggtaggccacctcgtccacgatgttgccgaagatggggtgccgctcgtgcttcttatcctcttccaccaggaaggactcttccagtctgtggaagaagctgtcgtccaccttggccatctcgttgctgaagatctcttgcagatagcagatccggttcttccgtctggtgtatcttcttctggcggttctcttcagccgggtggcctcggctgtttcgccgctgtcgaacagcagggctccgatcaggttcttcttgatgctgtgccggtcggtgttgcccagcaccttgaatttcttgctgggcaccttgtactcgtcggtgatcacggcccagcccacagagttggtgccgatggccaggccgatgctgtacttcttgtcgctgccgccgctgctgccgccactgctctctggtgttgctgactcgcttgttcctggtgtctcgctgccagaggatcctccgctagatcctccagacttcaggcctgtggcccacaggatgtggggtggcagcctctgatagtgacaagactgcagggcgattgtaaagaaggtcagctggggctgctttctccgcaggatattcagacaagggggcaggcccaggatgatgcaatacagctccagcacgtacaggcgcacccacaggtgtgggtacctaggccagtgggcctcgttgctaggagaataattcacgaagttccgccagcagtagccggactcctgctctgtcatgatctggatggtcacgccggagctgatcagatcgcgcaggccctgcctattccttgggtctgcgtggtgatacagcctggcgatgtagataaacagtgtcacgtgtggatatctggacaggaactcggtgatggccctgctacactctccgcaaggggaccagctcagaaaccatgtgatgctacatctggtattggggcagaagtaccgctctgtggtaaacttctcgatgaagttcacctccacgtgcttatttgtgttctgagaggtgtgcctccagatggagtgcctgcctccccagttgatctcgtacagcaggcatgtctcctttctcagctcccgggggtcaaagaacacttcaaactcgtgaggttcaatccggcggcgcagggttggatcgacggcgacaggcccagtctctgaggagactttccgcttcttctttggtgactcgaactcgcttccgtcggctgtccgtttcatggtggcggctctccctatagtgagtcgtattagcggccgcggatctctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatat

SEQ ID NO: 72 - protein, 1853 aa, Artificial Sequence; identical to SEQ ID NO: 1
  Description of Artificial Sequence: Synthetic polypeptide

SEQ ID NO: 73 - skipped

SEQ ID NO: 74 - protein, 1759 aa, Artificial Sequence
  Description of Artificial Sequence: Synthetic polypeptide
MGNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 75 - DNA, 21 bp, Unknown
  Description of Unknown: RNF2 target sequence
gcagttgtgtgacacggaagc

SEQ ID NO: 76 - DNA, 18 bp, Unknown
  Description of Unknown: RNF2 target sequence
catgttctaaaaatgtat

SEQ ID NO: 77 - DNA, 19 bp, Unknown
  Description of Unknown: RNF2 target sequence
gcatatgagacgtgtaaac

SEQ ID NO: 78 - DNA, 22 bp, Unknown
  Description of Unknown: VEGFA target sequence
ggggcatatgagacgtgtaaac

SEQ ID NO: 79 - DNA, 22 bp, Unknown
  Description of Unknown: HEK Site 3 target sequence
gcccagaagttggacgaaaagt

SEQ ID NO: 80 - RNA, 84 nt, Artificial Sequence
  Description of Artificial Sequence: Synthetic oligonucleotide
guuuuaguacucuguaaugaaaauuacagaaucuacuaaaacaaggcaaaaugccguguuuaucucgucaacuuguuggcgaga

SEQ ID NO: 81 - DNA, 20 bp, Artificial Sequence
  Description of Artificial Sequence: Synthetic oligonucleotide
gagtccgagcagaagaagaa

SEQ ID NO: 82 - DNA, 20 bp, Artificial Sequence
  Description of Artificial Sequence: Synthetic oligonucleotide
gtattcacctgaaagtgtgc

SEQ ID NO: 83 - DNA, 20 bp, Artificial Sequence
  Description of Artificial Sequence: Synthetic oligonucleotide
ggaatcccttctgcagcacc

SEQ ID NO: 84 - DNA, 20 bp, Artificial Sequence
  Description of Artificial Sequence: Synthetic oligonucleotide
gaacacaaagcatagactgc

SEQ ID NO: 85 - DNA, 20 bp, Artificial Sequence
  Description of Artificial Sequence: Synthetic oligonucleotide
ggcccagactgagcacgtga

SEQ ID NO: 86 - DNA, 20 bp, Artificial Sequence
  Description of Artificial Sequence: Synthetic oligonucleotide
ggcactgcggctggaggtgg

SEQ ID NO: 87 - DNA, 20 bp, Artificial Sequence
  Description of Artificial Sequence: Synthetic oligonucleotide
gcgtgacttccacatgagcg

SEQ ID NO: 88 - DNA, 20 bp, Artificial Sequence
  Description of Artificial Sequence: Synthetic oligonucleotide
gactcacccaggagtgcgtt

SEQ ID NO: 89 - DNA, 20 bp, Artificial Sequence
  Description of Artificial Sequence: Synthetic oligonucleotide
ggcactcgggggcgagagga

SEQ ID NO: 90 - DNA, 20 bp, Artificial Sequence
  Description of Artificial Sequence: Synthetic oligonucleotide
gagctcactgaacgctggca

SEQ ID NO: 91 - DNA, 20 bp, Artificial Sequence
  Description of Artificial Sequence: Synthetic oligonucleotide
gtcatcttagtcattacctg

SEQ ID NO: 92 - DNA, 20 bp, Artificial Sequence
  Description of Artificial Sequence: Synthetic oligonucleotide
gaccccctccaccccgcctc

SEQ ID NO: 93 - protein, 4 aa, Artificial Sequence
  Description of Artificial Sequence: Synthetic peptide
GGGS

SEQ ID NO: 94 - protein, 5 aa, Artificial Sequence
  Description of Artificial Sequence: Synthetic peptide
GGGGS

SEQ ID NO: 95 - RNA, 60 nt, Artificial Sequence; identical to SEQ ID NO: 62
  Description of Artificial Sequence: Synthetic oligonucleotide

SEQ ID NO: 96 - protein, 6 aa, Artificial Sequence
  Description of Artificial Sequence: Synthetic 6xHis tag
HHHHHH

SEQ ID NO: 97 - protein, 16 aa, Artificial Sequence
  Description of Artificial Sequence: Synthetic peptide
  MISC_FEATURE (1)..(16): This region may encompass 2-4 “Gly Gly Gly Ser” repeating units
  See specification as filed for detailed description of substitutions and preferred embodiments
GGGSGGGSGGGSGGGS

SEQ ID NO: 98 - protein, 20 aa, Artificial Sequence
  Description of Artificial Sequence: Synthetic peptide
  MISC_FEATURE (1)..(20): This region may encompass 2-4 “Gly Gly Gly Gly Ser” repeating units
  See specification as filed for detailed description of substitutions and preferred embodiments
GGGGSGGGGSGGGGSGGGGS

SEQ ID NO: 99 - protein, 4 aa, Artificial Sequence
  Description of Artificial Sequence: Synthetic peptide
TTER

SEQ ID NO: 100 - DNA, 21 bp, Artificial Sequence
  Description of Artificial Sequence: Synthetic oligonucleotide
cgcaaatgggcggtaggcgtg

SEQ ID NO: 101 - DNA, 20 bp, Artificial Sequence
  Description of Artificial Sequence: Synthetic oligonucleotide
taatacgactcactataggg

SEQ ID NO: 102 - protein, 1841 aa, Artificial Sequence
  Description of Artificial Sequence: Synthetic polypeptide
PKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 103 - protein, 1526 aa, Artificial Sequence
  Description of Artificial Sequence: Synthetic polypeptide
MDQDIALRLVSDHFFDFFGQHQDALLDDQLLACLLLDDVRQVGAASRSSGSSAQHLDLILAVRILDDQGPRLILGGVRGQQHHILICGLVVGGVHQDVRLWLVADHFLHLLRQHQDGLLNDQLFPSLLLNDVAQISAPPAPAAPAAVTSQLRQVDPCLVQAGDALVDQGGVQHLFGAGVPLPVDGGVKVLEGGRGSQIGQGKQVDDILGLLSDGLIPVLVVGGQHFVQISVGQDHSLGELADLLDDLVQVVLVLFHKQLFLLIILGGALQLLIVAGQVQEVHIFGGQGQFVSLLQFAGRGQHSLPAVFQLEQGVLRQLDDQVLFHFFVALGFQKVDGILLEAASFHDGDPQQLFHTLQFLGLALFHFGHHQHRIGHGGAVEAAVLLRVPVLLSGDQLIAVPLGQDRLFAEAACLHLGLFHDIHLGHAQHFPHGGKIPALIPHDLPGFAVCLDQRPLPDLAVGQGNLGLEKVHDVAVEEVLGGSLADFLLALGDHLPHVVHLVVAVHELAFQLRVLFDQGGSHDGVQVGVVGVVVVVDLAHFVKLEILPEIGHQLGLQGDHFHFPDQLVILVVLSVHPGVQDLCHVLCDLPGFHQLSLDEAGLIQFAQAASLGLGQIVELSLGNQLGVQQLPPVVLHLLHDLFGGHVVALAPVLVASGQHLVVDGVVLQKALRHDMVHIVVGQPVDVQFLVHVHIPPILQVVQVQLLVLQLGVFHGVFFQDLAAQLFDALFDPLHSLAAVLLSLLGGLVLSGHFDHDVLGLVPAHHFHELVHHLHCLQDALLNGGAAGQIGNVLVQAIALAGHLGFLDVLFKGQAVVVDQLHEVSVGEAVGLQEIQDCLAGLLVPDAVDQLPAQPAPAGVSPPLQLLHHFVVEQVGIGFQPFLDHLSVLKQCQGQHDIFQNVLVFLIVQEVLVLDNFQQIVVCAQGGVEPIFHAGDFHGVEALDFLEVVLFQLLHGHFPVGLEQQVHDGLFLLAAQEGGLSHSLGHVFHFGQLVIHGEVLVQQAVLGQHLLVGQVLIEVGHPLDEALGGSALVHHFLEVPGGDGFLALSGHPGESAVSPGQRAHVVGDAEGQDLLDLFPVVLQEWVKIFLPPQNGVQLSQVDLVGDAAVVEGPLLPQQVLSVQLHEQFLGAVHLFQDGLDELVELFLAGSAVNVAGVAVLALVEENLFVLLRQLLPHESFQQGQVLVVLVVSLDHRGAQGGLGDLGVHSQDVAQQDGVGQVLGGQKQVGVLVADLGQQVVQVVVVGVLAQLQFGILGQVEVALEVGGQAQAQGNQVSEQAILLLAGQLGDQIFQPSALAQSGRQDGLGVHAAGVDGVFLEQLVVGLHQLDEQLVHVAVVGVQVALDQEVAPELDHVGQGQIDQPQVGLVGAVHQFLSQVVDGGVLLVVGHLVHDVAEDGVPLVLLILFHQEGLFQSVEEAVVHLGHLVAEDLLQIADPVLPSGVSSSGGSLQPGGLGCFAAVEQQGSDQVLLDAVPVGVAQHLEFLAGHLVLVGDHGPAHRVGADGQADAVLLVAAAAAAATALWCC

SEQ ID NO: 104 - protein, 149 aa, Artificial Sequence
  Description of Artificial Sequence: Synthetic polypeptide
MPSSIRFILSRLFFCPFWVVWFSLAISITMFSGLCRPITFTSSSTTFTVCRMPFLMAGLPARLAMCSCRLSPWPDTWAFWMSSLKVRLSSWISCMKFLLAKPSDFRKSRIVLPDCLSRMPLISFRLSLPQPVYLRRFSCFITLSSNRWA

SEQ ID NO: 105 - DNA, 18 bp, Artificial Sequence
  Description of Artificial Sequence: Synthetic oligonucleotide
tagaaggcacagtcgagg

SEQ ID NO: 106 - DNA, 17 bp, Artificial Sequence
  Description of Artificial Sequence: Synthetic oligonucleotide
caggaaacagctatgac

SEQ ID NO: 107 - DNA, 23 bp, Artificial Sequence
  Description of Artificial Sequence: Synthetic oligonucleotide
agcggataacaatttcacacagg

SEQ ID NO: 108 - DNA, 18 bp, Artificial Sequence
  Description of Artificial Sequence: Synthetic oligonucleotide
agcgagtcagtgagcgag

SEQ ID NO: 109 - DNA, 20 bp, Artificial Sequence
  Description of Artificial Sequence: Synthetic oligonucleotide
gggaaacgcctggtatcttt

SEQ ID NO: 110 - protein, 286 aa, Artificial Sequence
  Description of Artificial Sequence: Synthetic polypeptide
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 111 - DNA, 20 bp, Artificial Sequence
  Description of Artificial Sequence: Synthetic oligonucleotide
ataataccgcgccacatagc

SEQ ID NO: 112 - DNA, 20 bp, Artificial Sequence
  Description of Artificial Sequence: Synthetic oligonucleotide
cggcatcagagcagattgta